(19) United States
(12) Patent Application Publication
(10) Pub. No.: US 2024/0410876 A1
(43) Pub. Date: Dec. 12, 2024

(54) EXTRACELLULAR VESICLE DRUG ANALYSIS FOR REAL-TIME MONITORING OF TARGETED THERAPY

(71) Applicant: NATIONAL UNIVERSITY OF SINGAPORE, Singapore (SG)

(72) Inventors: Huilin SHAO, Singapore (SG); Sijun PAN, Singapore (SG); Yan ZHANG, Singapore (SG); Auginia NATALIA, Singapore (SG)

(21) Appl. No.: 18/264,340

(22) PCT Filed: Feb. 8, 2022
(86) PCT No.: PCT/IB2022/051088

§ 371 Date: Aug. 4, 2023

Related U.S. Application Data
(60) Provisional application No. 63/147,170, filed on Feb. 8, 2021.

(51) Int. Cl. G01N 33/50; B82Y 15/00; C12Q 1/28; G01N 27/327; G01N 33/574; G01N 33/58

(52) U.S. Cl. CPC G01N 33/5023; C12Q 1/28; G01N 27/3278; G01N 33/57423; G01N 33/57492; G01N 33/58; B82Y 15/00; G01N 2333/71; G01N 2333/908; G01N 2500/02

(57) ABSTRACT

This application provides methods and compositions related to real time monitoring targeted therapeutics by measuring time-dependent dynamics in distinct subpopulations of secreted vesicles. The technology utilizes bio-orthogonal probe amplification and spatial patterning of molecular reactions within plasmonic resonators to measure EV drug dynamics directly in patient blood samples. Small-molecule click probes are used in the assays for competitive, in situ target labeling in whole vesicles; the labeling of target by the probes can be enzymatically amplified to detect drug occupancy in EVs.

### CROSS-REFERENCE TO RELATED APPLICATION

This application claims the benefit of International Patent Application No.: PCT/IB2022/051088, filed Feb. 8, 2022 which claims priority to U.S. Provisional Application No. 63/147,170, filed on Feb. 8, 2021, and titled “EXTRACELLULAR VESICLE DRUG ANALYSIS FOR REAL-TIME MONITORING OF TARGETED THERAPY,” the content of which is herein incorporated by reference in its entirety for all purposes.

### FIELD

The present invention relates to production of nucleic acids or polypeptides in a cell free system.

### BACKGROUND

During the past decade, the paradigm of cancer treatment has evolved from nonspecific cytotoxic agents to personalized, mechanism-driven targeted therapeutics (Moscow et al. (2018)). Unlike conventional chemotherapies which interfere with all rapidly dividing cells, targeted therapies interact with distinct molecular targets that are crucial to tumor survival to induce striking regressions. Despite the specific nature of targeted drugs, current clinical evaluation of their treatment in solid tumors relies primarily on tumor volumetric imaging (Eisenhauer et al. (2009); Litière et al. (2017)), which is delayed and insensitive to drug molecular interactions. These evaluation methods also lack molecular specificity, requires a large amount of sample, and requires complex sample processing, especially for solid tumor assessment.

Certain new analytical technologies are in development to measure specific drug-target interactions (Basu et al. (2013)). For example, thermal shift assay has been developed to evaluate drug-protein interactions, by measuring changes in the thermal stability of drug-target complexes (Martinez Molina et al. (2013)); as such changes are subtle and nonspecific, the approach could only evaluate high-abundance proteins that display large stability shifts. Chemical proteomics use chemically modified small molecule probes to enrich for interacting proteins, which can improve the detection coverage and (Jones et al. (2017); Gerry et al. (2018)); however, the approach too requires extensive processing and a large sample amount. Due to the complexity of these analytical technologies, drug-target interactions are not commonly measured nor longitudinally assessed in patients undergoing clinical cancer treatment; in monitoring of solid tumors, such analyses are particularly challenging due to the need for repeat invasive tissue biopsies.

### BRIEF SUMMARY

In one aspect, this disclosure provides a method of measuring the binding of a drug to a target in a subject that has been treated with a drug over a treatment period, wherein the method comprises: contacting a probe with extracellular vesicles (EVs) from samples obtained from the subject at different time points of the treatment period, wherein the probe is capable of competing with the drug in binding to the target in the EVs, and detecting the binding of the probe to the EVs in the samples, wherein a decrease in the binding of the probe to the EVs as treatment period progresses indicates an increase in the binding of the drug to the target in the subject.

In some embodiments, contacting the probe with EVs from the samples obtained from the subject comprises: for each sample, i) contacting the EVs from the sample with a sensor, wherein the EVs are captured to the sensor, ii) contacting the probe with the EVs captured on the sensor, wherein the probe binds to target molecules on the EVs that are not already bound by the drug, wherein the probe's binding to the target molecules results in a signal P. In some embodiments, the signal P is in situ enzymatic amplification of signal corresponding to the binding of the probe to the target molecules. In some embodiments, contacting the EVs with the sensor results in a signal M, and wherein the method further comprises determining the binding of the drug to the target based on the signal P and the signal M.

In another aspect, this disclosure provides a method of comparing the potency of a first drug relative to a second drug on a subject comprising: contacting the first drug and the second drug with extracellular vesicles (EVs) obtained from a sample of the subject separately, adding a probe to the EVs that have been contacted with the first drug and to the EVs that have been contacted with the second drug, wherein the probe is capable of competing with both the first drug and the second drug in binding to the target molecules in the EVs, and detecting the binding of the probe to the EVs that have been contacted with the first drug and EVs that have been contacted with the second drug, wherein a lower binding of the probe to the EVs that have been contacted with the first drug relative to the second drug indicates that the first drug is more potent than the second drug, and wherein a higher binding of the probe to the EVs that have been contacted with the first drug relative to the second drug indicates that the first drug is less potent than the second drug in the samples.

In some embodiments, the contacting the EVs with varying increasing concentrations of a first drug and a second drug, contacting the EVs that have been contacted with varying increasing concentrations of the first or the second drug with a probe, determining the drug occupancy at each concentration of the first and second drug, determining an IC₅₀ of the drug occupancy of the first drug and an IC₅₀ of the drug occupancy of the second drug, determining that the first drug is more potent than the second drug if the IC₅₀ of the drug occupancy of the first drug is lower than that of the second drug, or determining the first drug is less potent than the second drug if the IC₅₀ of the drug occupancy of the first drug is higher than that of the second drug.

In another aspect, this disclosure provides a method of detecting mutations in EGFR in a subject, the method comprising: i) contacting an EV sample from a patient with a drug that targets the wild type EGFR, ii) adding a probe to the EVs samples that have contacted with the drug, wherein the probe is capable of competing with the drug in binding to the wild type EGFR iii) determining an IC₅₀ of drug occupancy for EVs from the patient as compared to that of control EVs expressing the wild type EGFR, and iv) determining that the subject has a mutation in the EGFR if the IC₅₀ of drug occupancy for the EV sample from the patient is less than the IC₅₀ of drug occupancy for the control EVs.

In some embodiments, the EVs are captured on the sensor via binding to a capture agent immobilized on the sensor. In some embodiments, the capture agent is an antibody that is against one or more proteins selected from the group consisting of CD63, CD81, CD9, HER2, LAMP-1, Alix, HSP90, an Flotillin 1, a TSG101, EGFR, EpCAM, and MUC1.

In another aspect, the disclosure provides a method of diagnosing a lung cancer in a subject, the method comprising: contacting a probe with extracellular vesicles (EVs) from a sample obtained from the subject, wherein the EVs are captured by a capture agent immobilized on a sensor, wherein the capture agent binds to a cancer marker on the EVs, wherein the cancer marker is preferentially expressed in lung cancer than normal cells, wherein the probe binds to EGFR on the EVs, wherein the binding of the capture agent to the cancer marker does not substantially interfere with the binding of the probe to the cancer marker on the EVs, detecting a signal associated with binding of the probe to the EVs, and determining subject has the lung cancer if the signal is greater than a control. In some embodiments, the cancer marker is selected from the group consisting of MUC1, EpCAM, and EGFR.

In another aspect, this disclosure provides a sensing element comprising nanogap structures patterned on a conductive layer that is deposited on a glass substrate, wherein the nanogap structures are patterned to form nanogaps between adjacent nanostructures, and wherein the average size of nanogap is 20 to 500 nm, wherein illumination of the nanogap structures produces a surface plasmon resonance. In some embodiments, the nanogap structures are nanorings, wherein the nanogaps are formed between an outer circular shape and an inner circular shape wherein the outer circular shape has an outer diameter in a range from 200 nm to 500 nm, and/or the inner circular shape has an inner diameter in a range from 30 nm to 250 nm. In some embodiments, the conductive layer comprises a material selected from the group consisting of a silver, gold, copper, titanium, aluminum, and chromium.

In some embodiments, this disclosure provides a sensor comprising an array of any of the sensing element described above.

In another aspect, this disclosure provides a microfluidic system comprising: a flow cell, wherein the flow cell comprises a sensor array comprising a plurality of sensing elements of embodiment 29, microfluidic channels for introducing samples into the sensor array; and a light source, wherein the light source is arranged to illuminate the sensor array.

In yet another aspect, this disclosure provides a probe that is capable of competing with a drug in binding to its target, wherein the probe contains a tag, wherein the tag can ligate to an enzyme, and wherein the enzyme is capable of catalyzing a reaction to produce an insoluble optical product and producing a detectable signal. In some embodiments, the probe is a click probe. In some embodiments, the click probe ligates to the enzyme through a copper-free click reaction. In some embodiments, the enzyme is conjugated to tetrazine or dibenzocyclooctyne (DBCO). In some embodiments, the enzyme is tetrazine-conjugated horseradish peroxidase (HRP).

In some embodiments, the EGFR inhibitor disclosed herein is selected from the group consisting of afatinib, osimertinib, erlotinib, dacomitinib, CNX2006, and WZ4002.

### BRIEF DESCRIPTION OF THE DRAWINGS

FIG. 1 A-1D shows ExoSCOPE for activity-based analysis of EV drug dynamics. FIG. 1A.

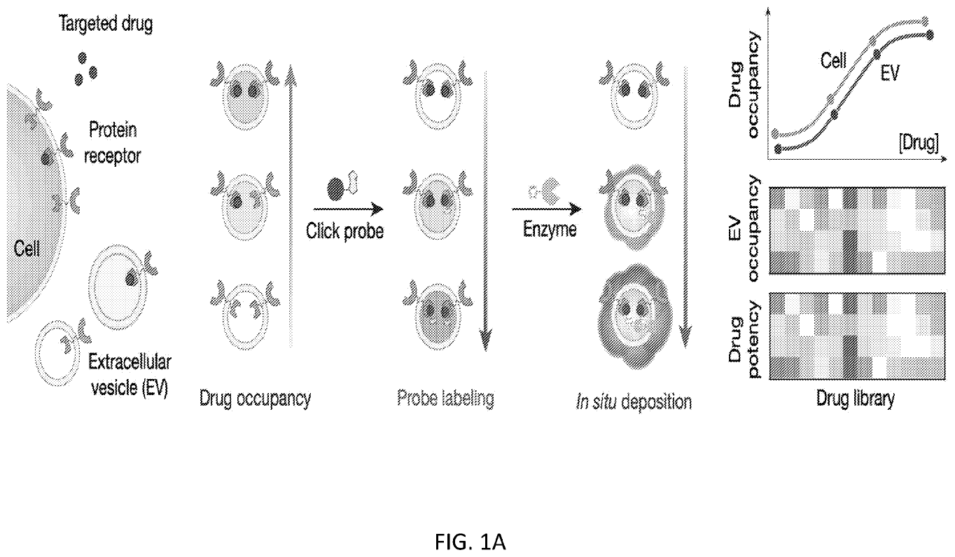

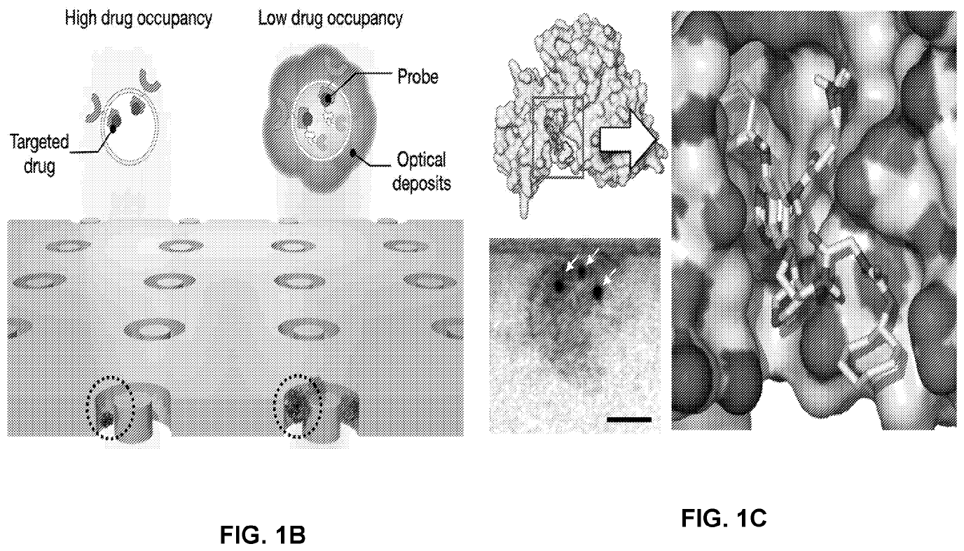

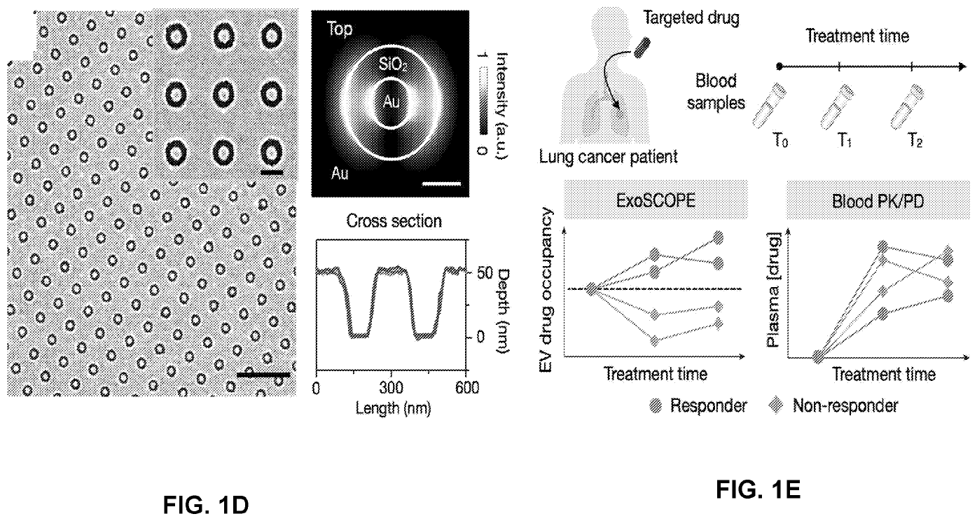

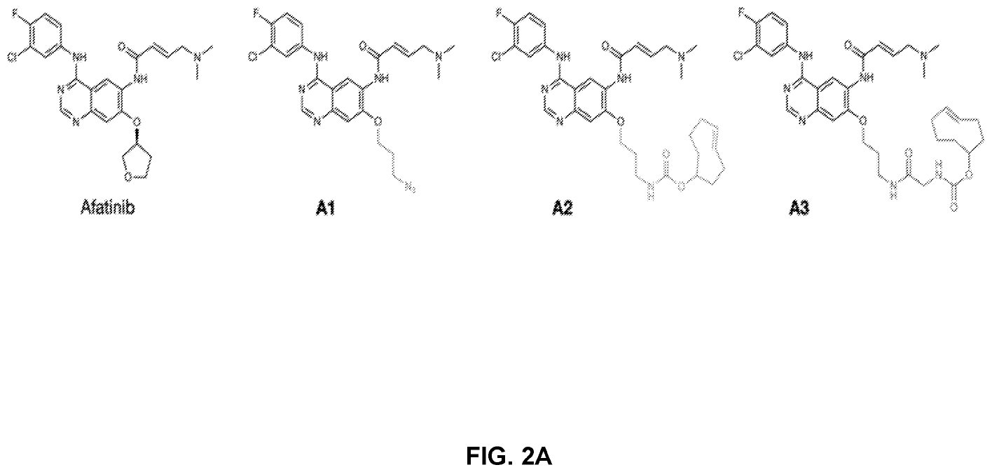

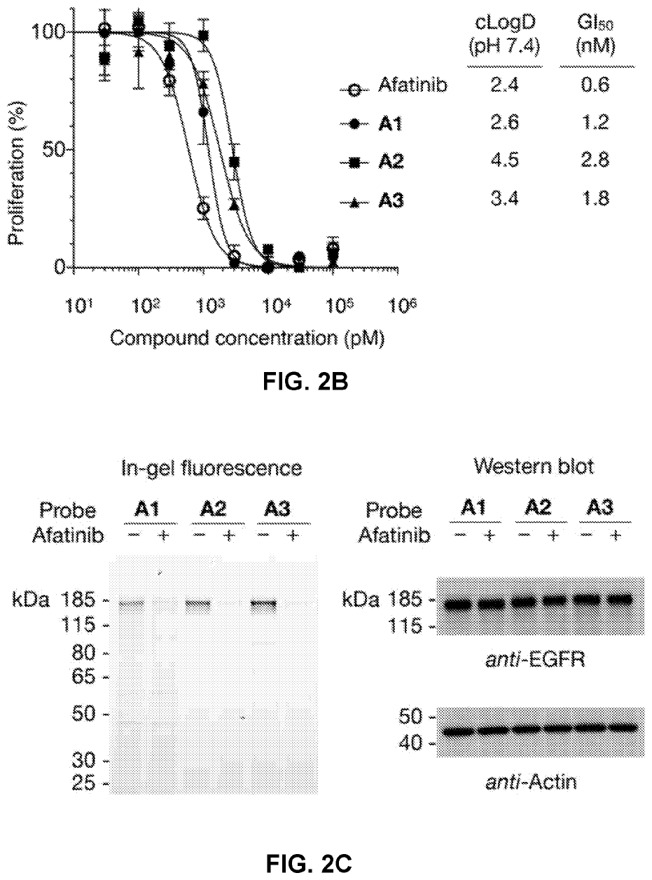

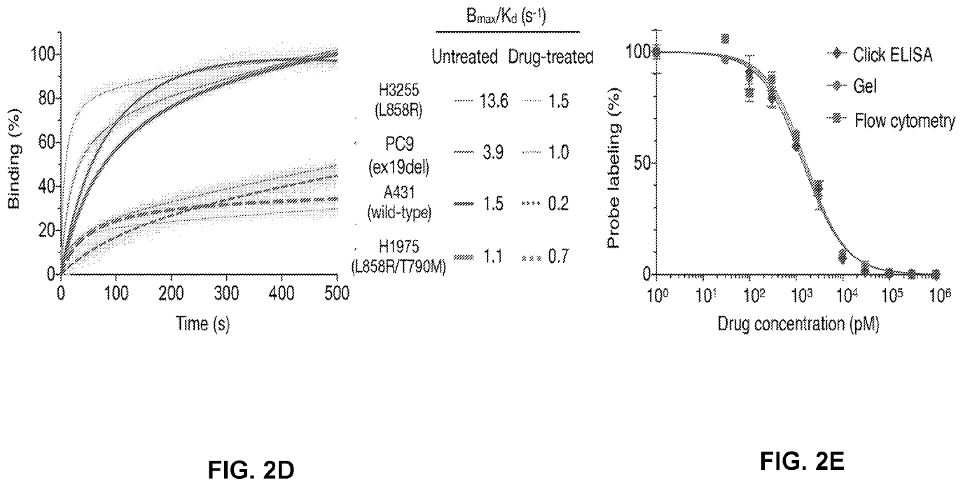

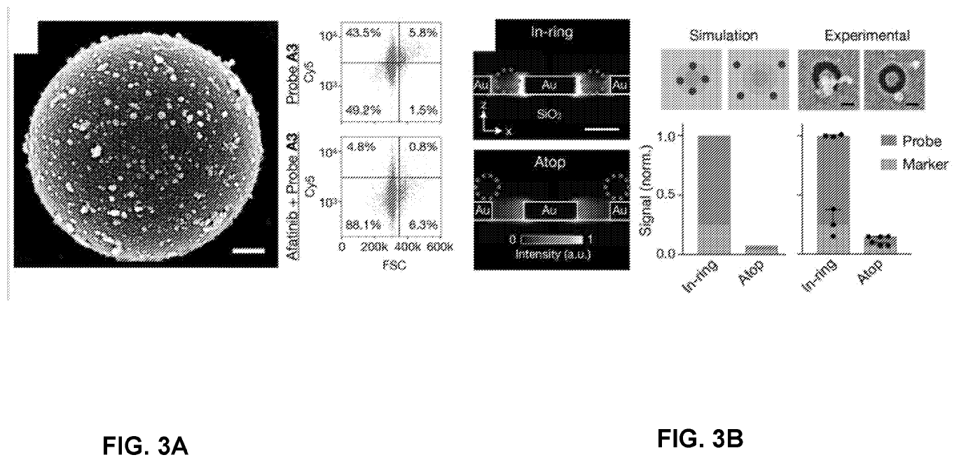

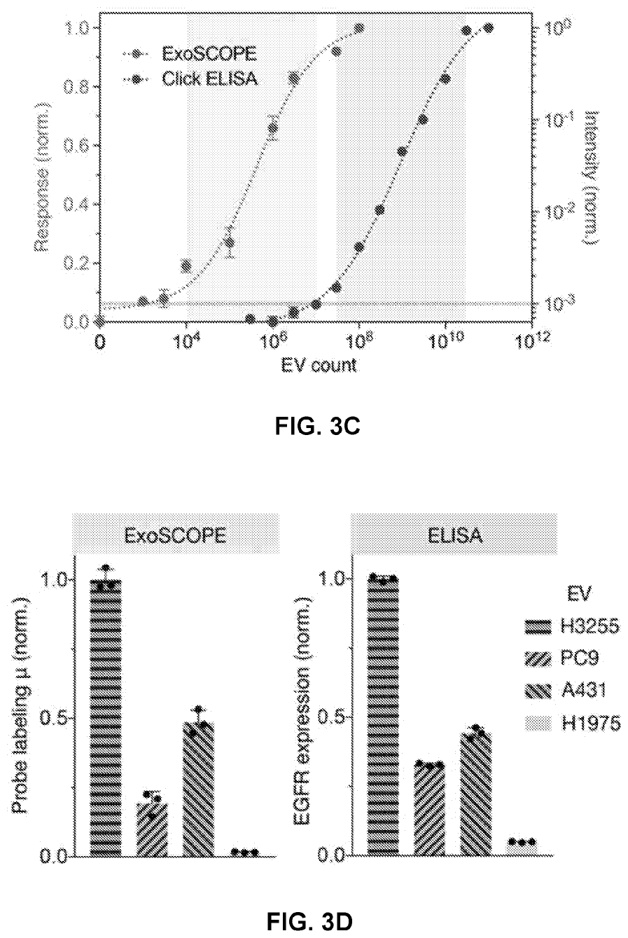

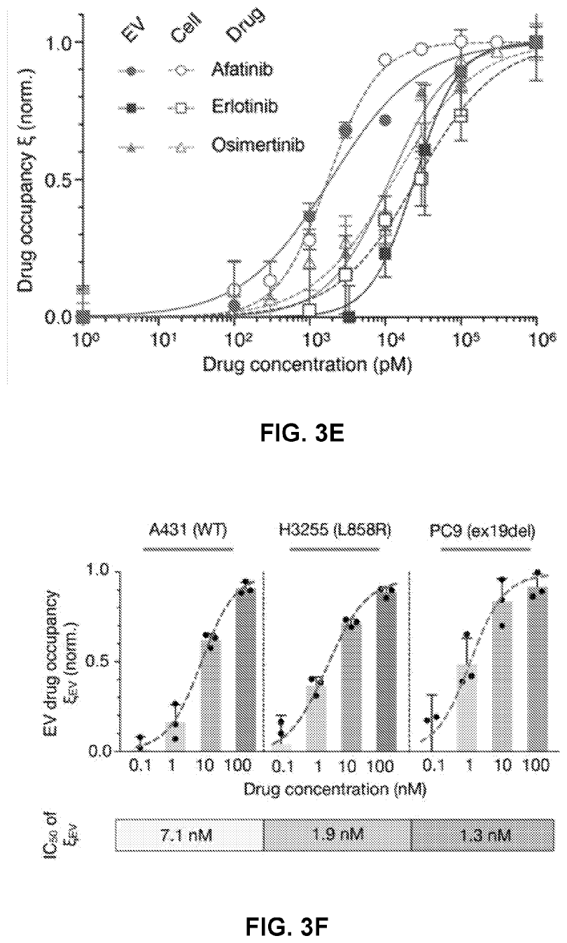

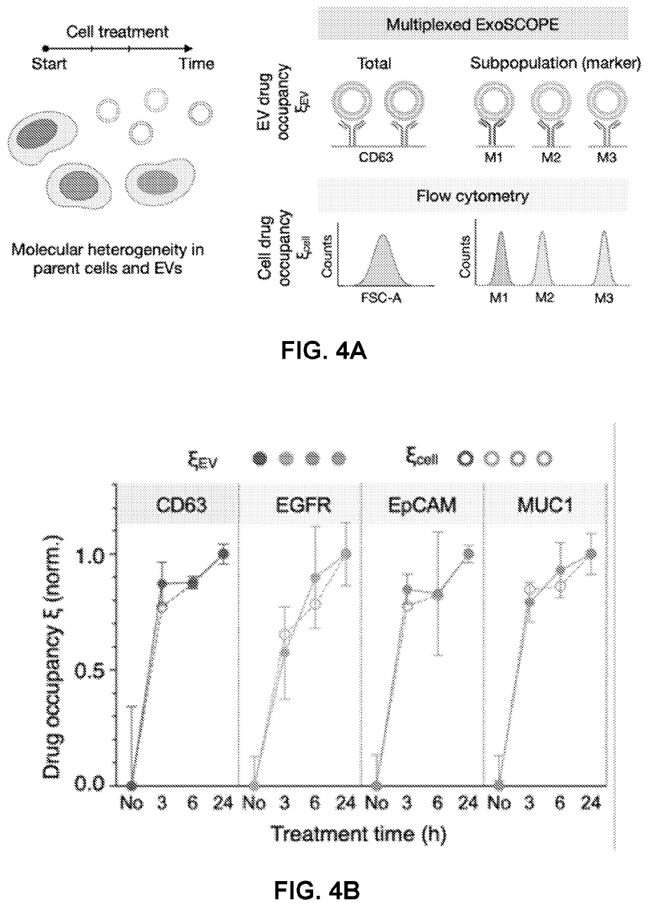

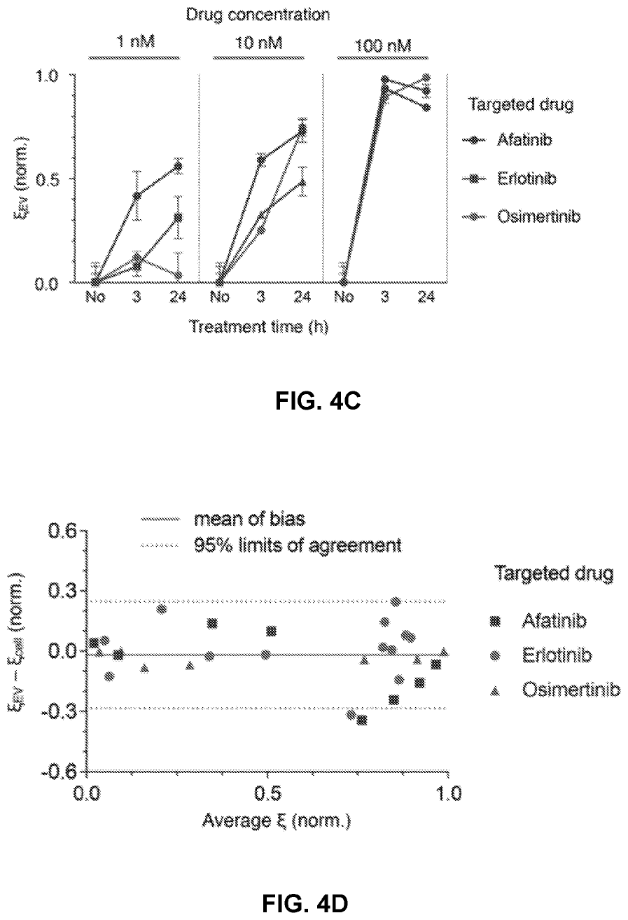

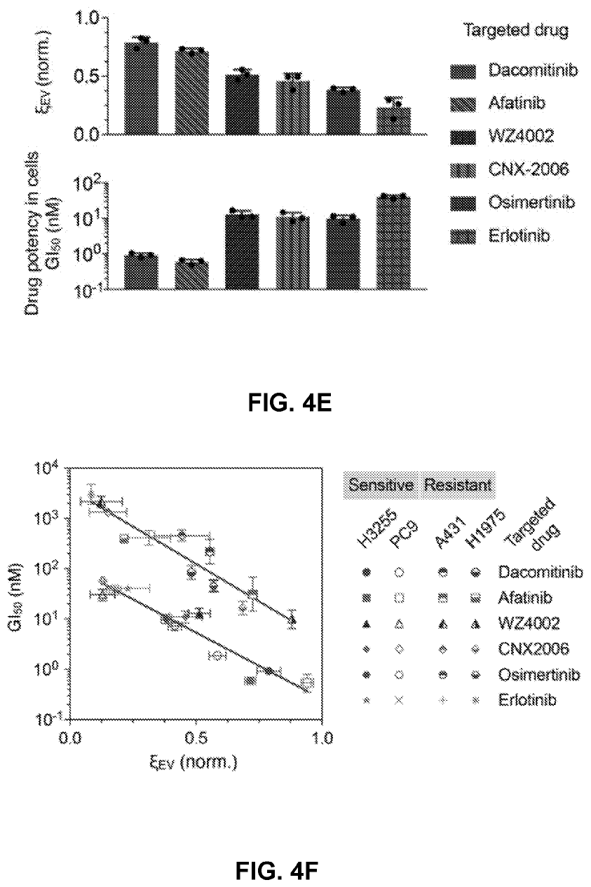

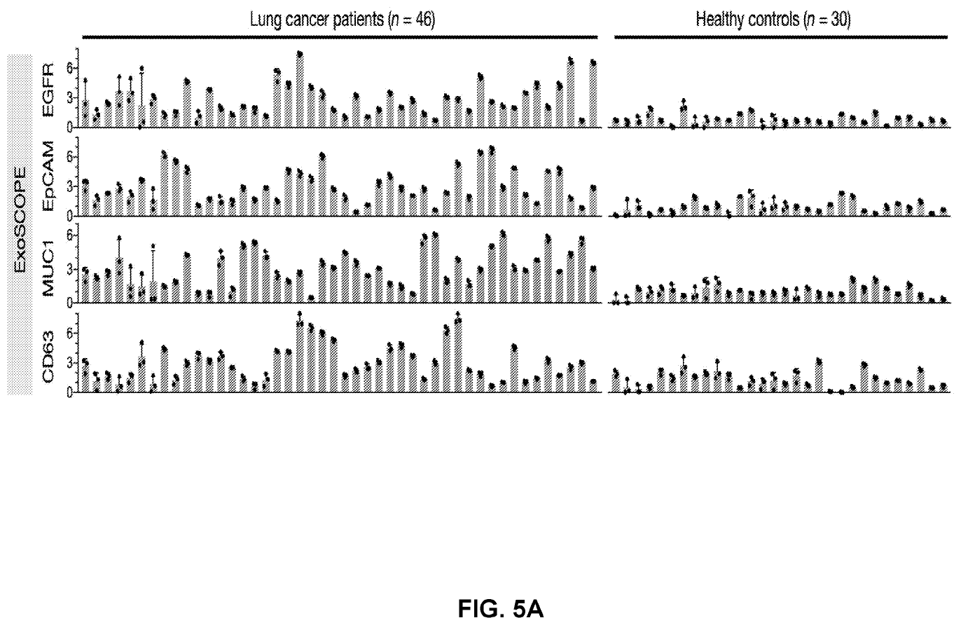

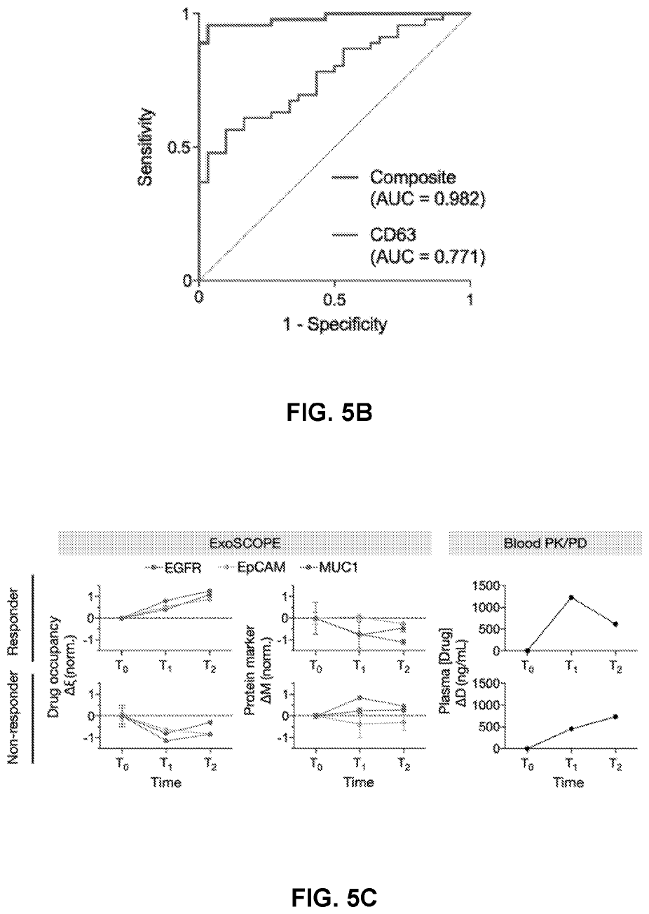

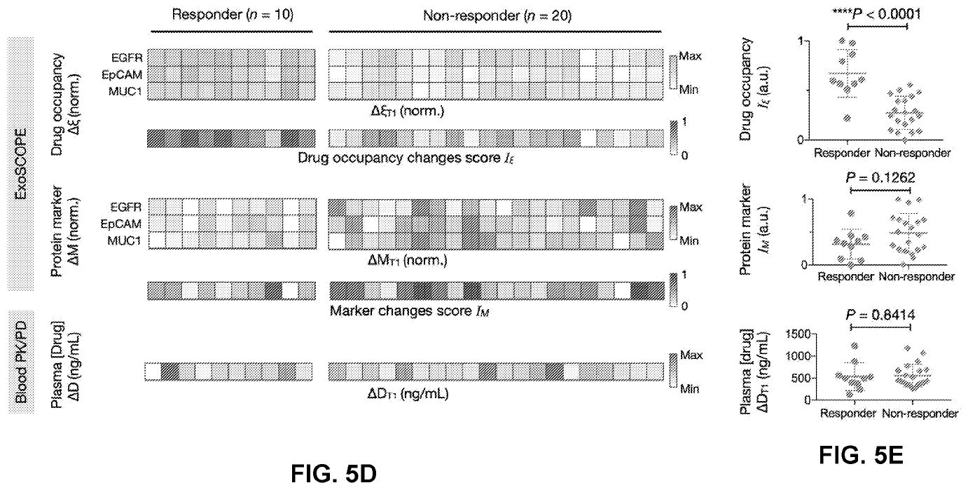

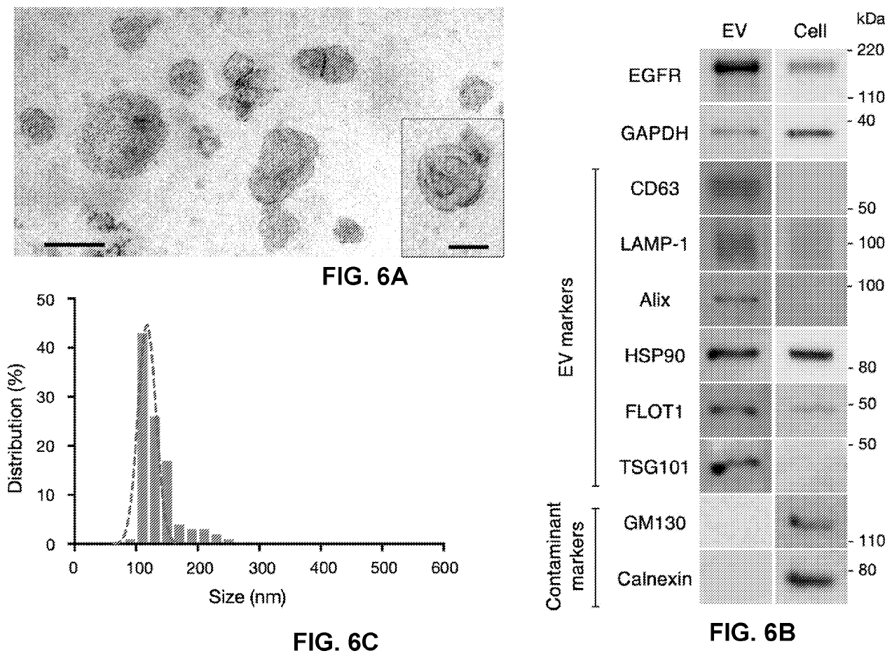

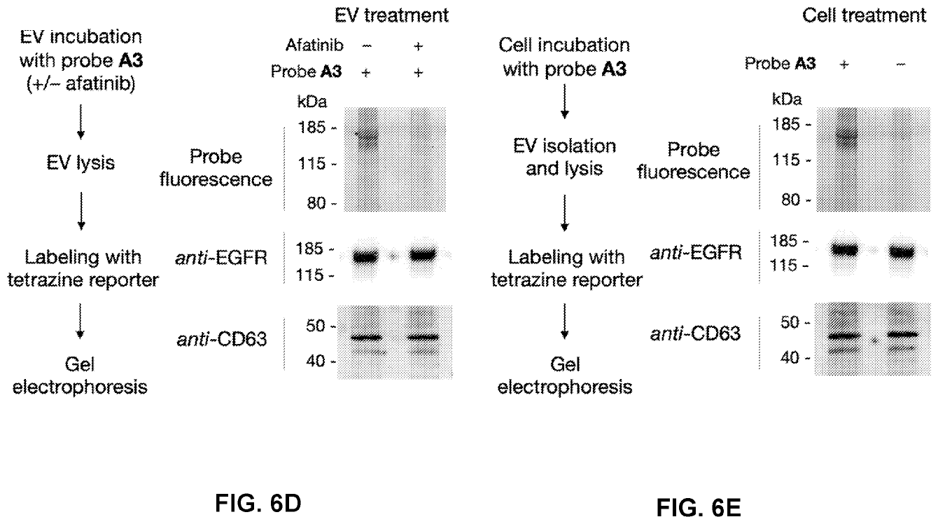

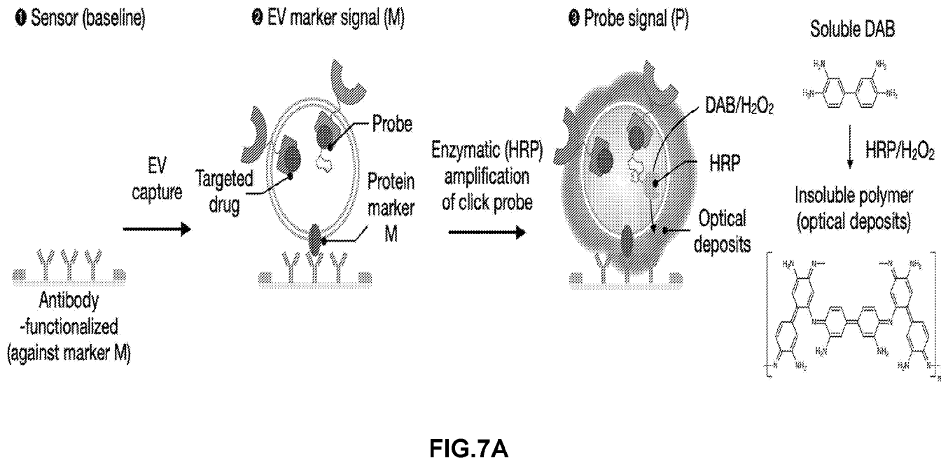

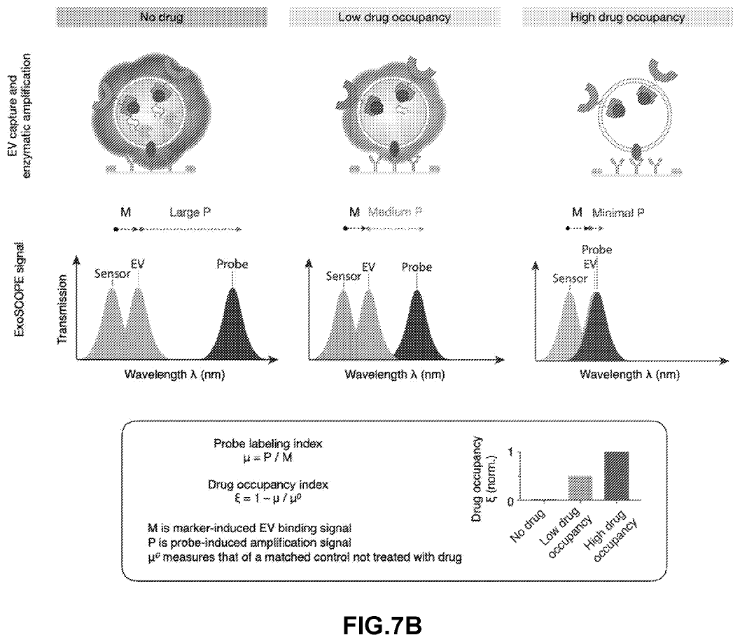

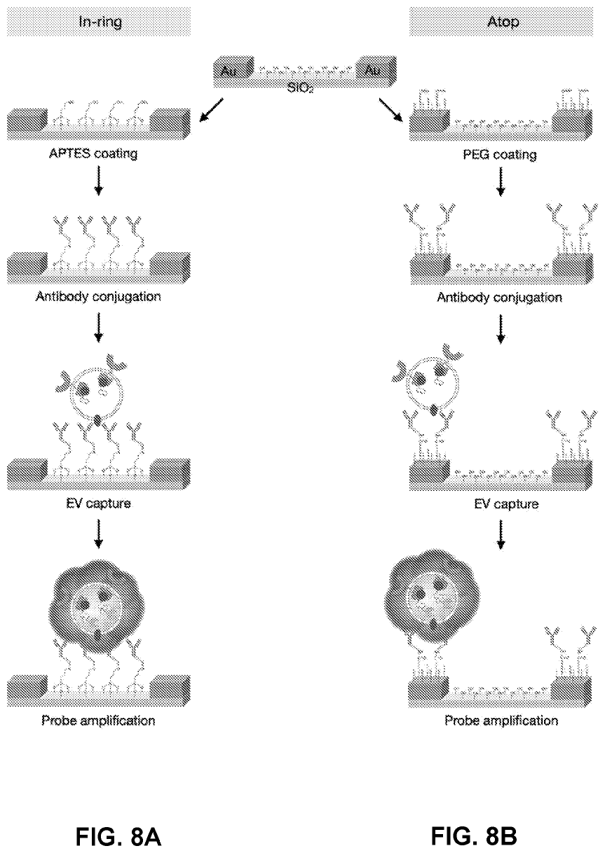

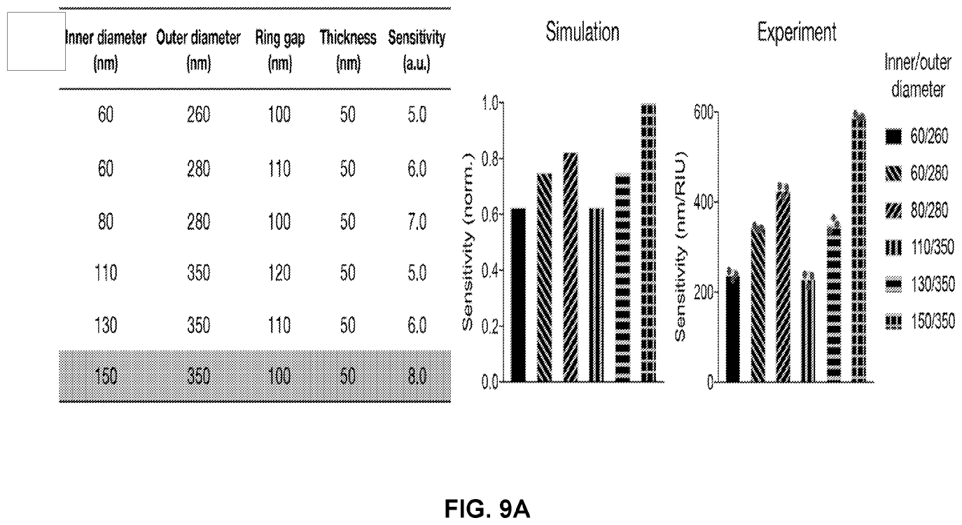

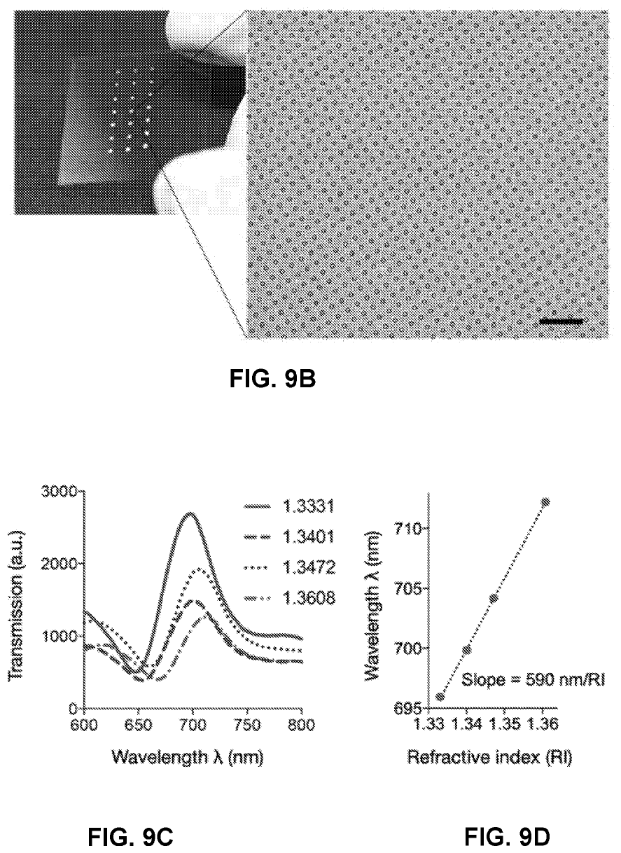

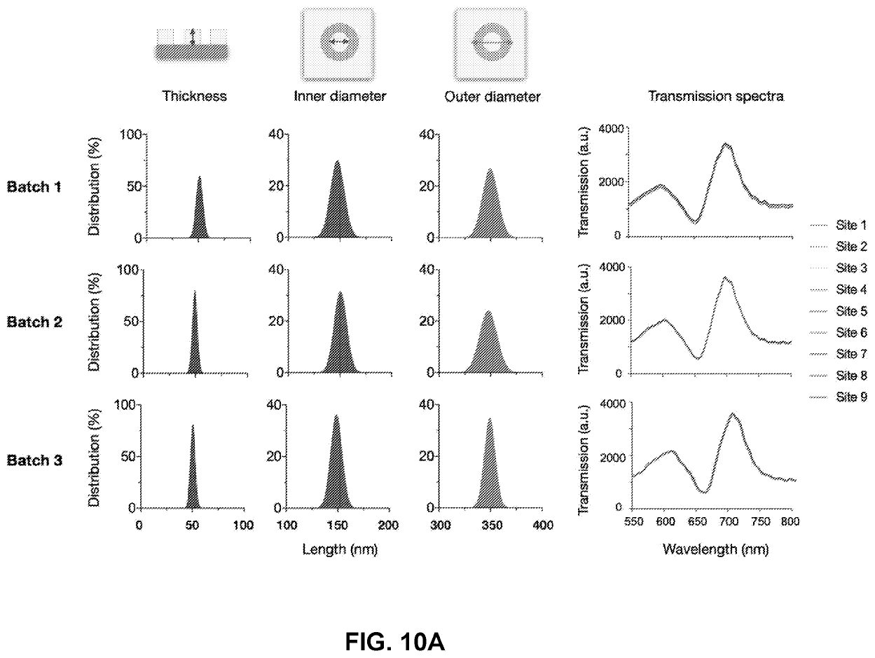

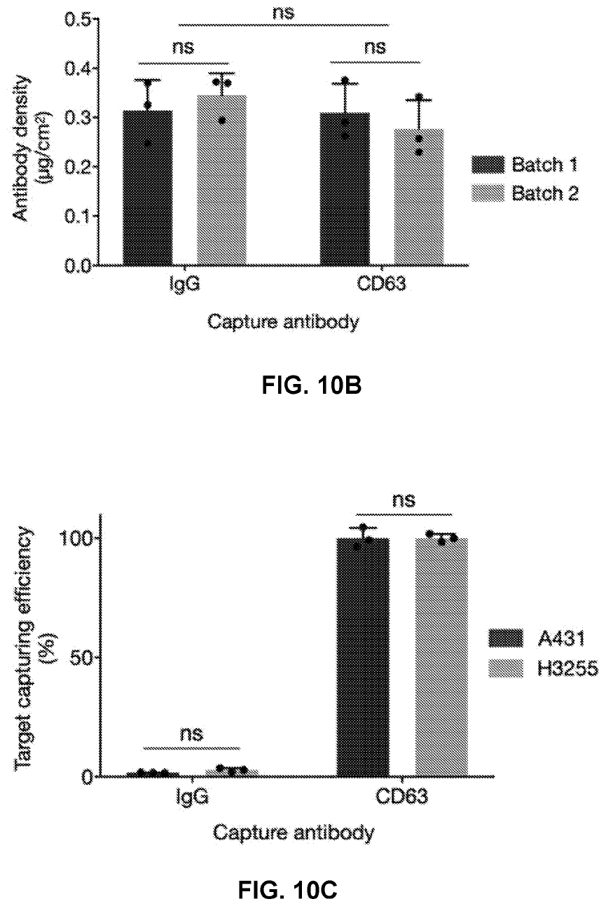

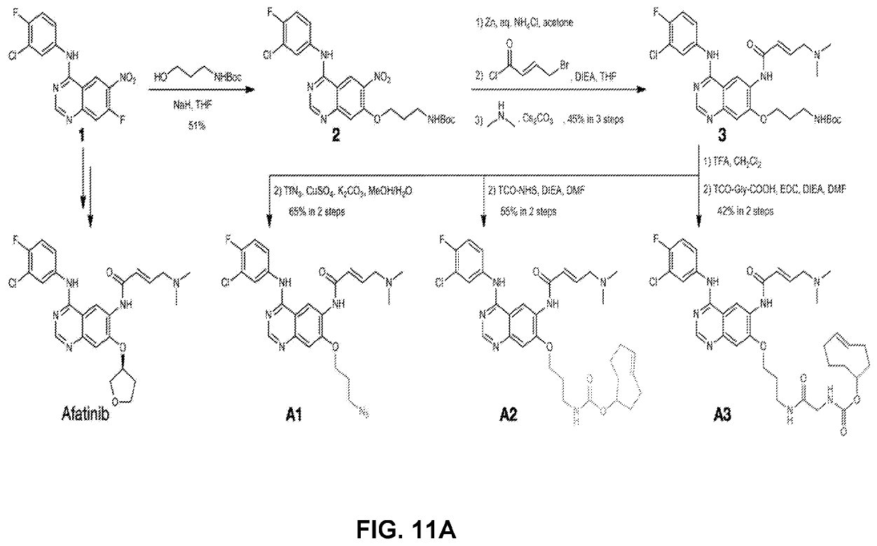

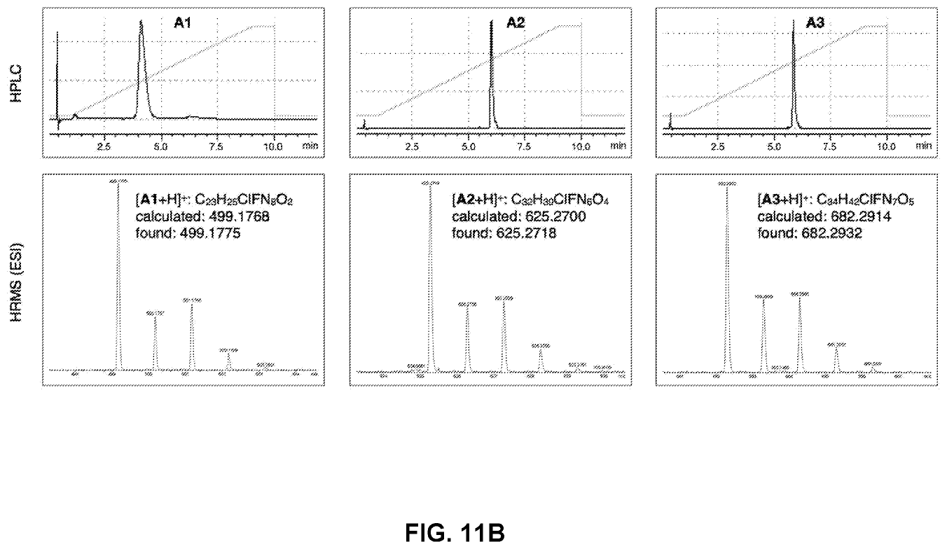

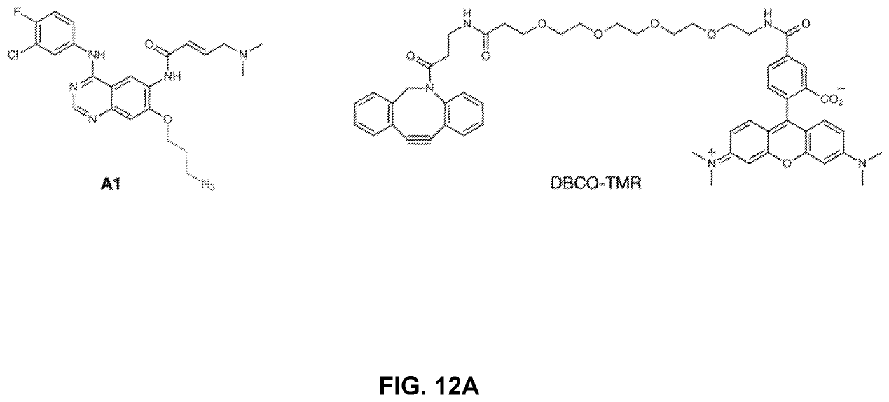

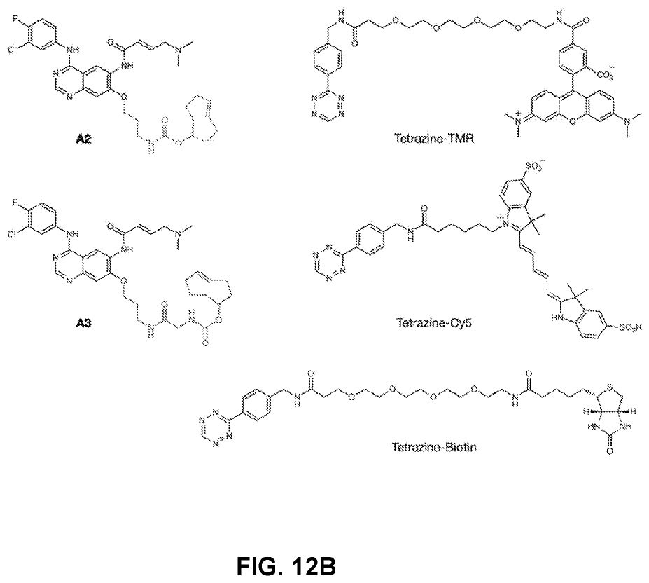

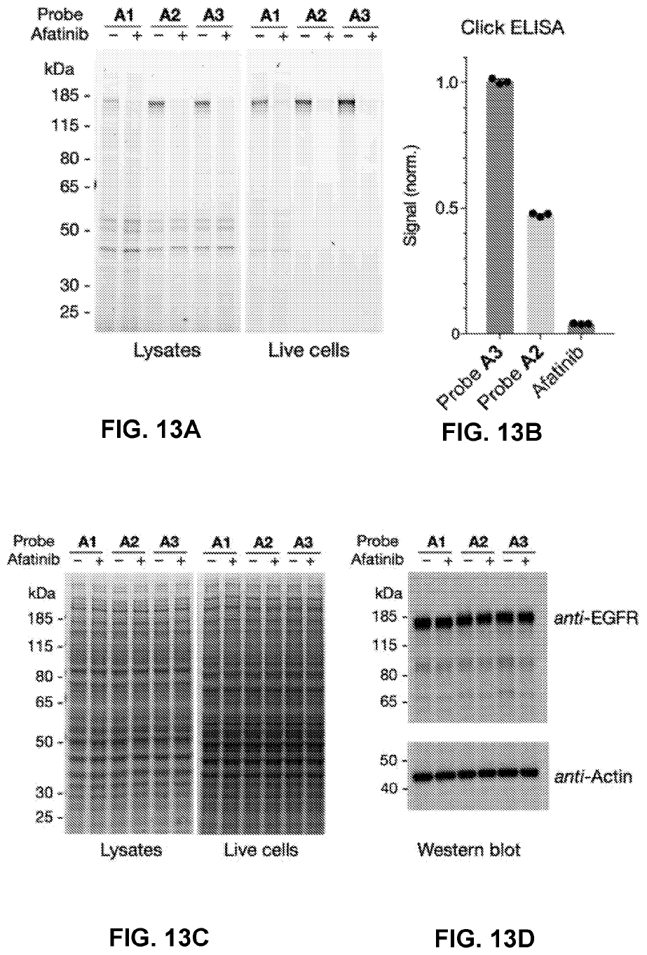

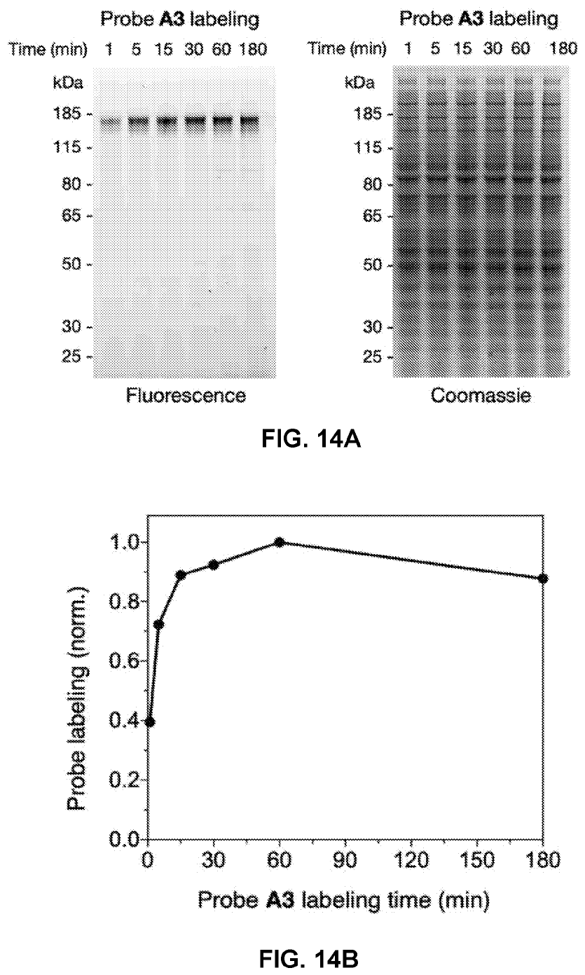

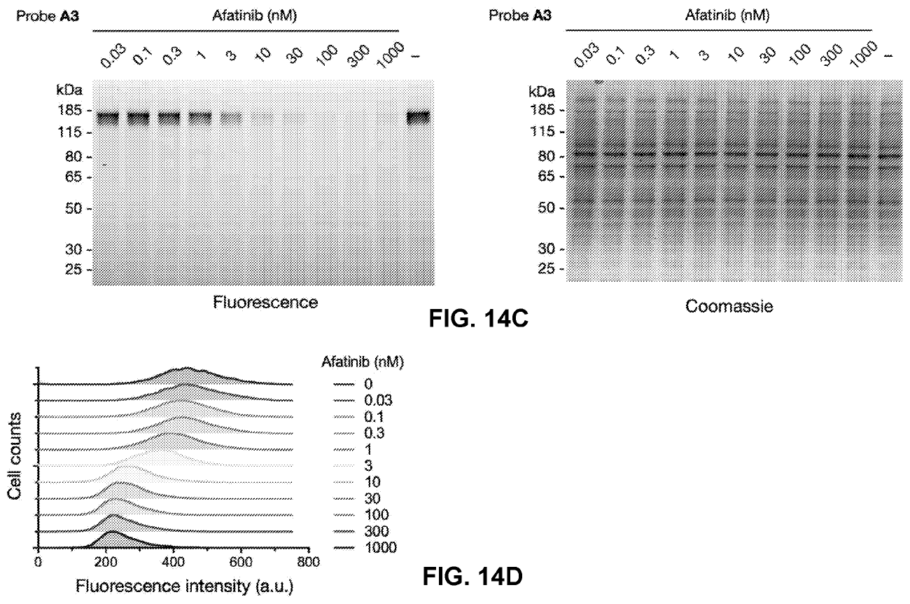

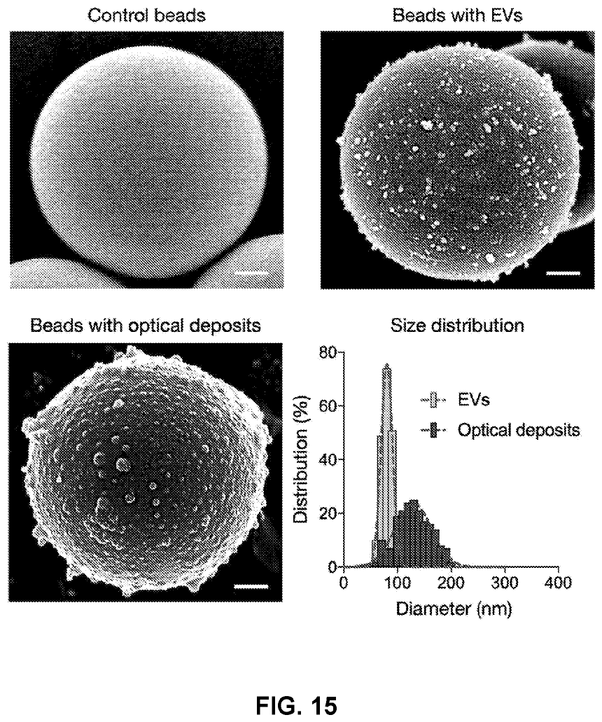

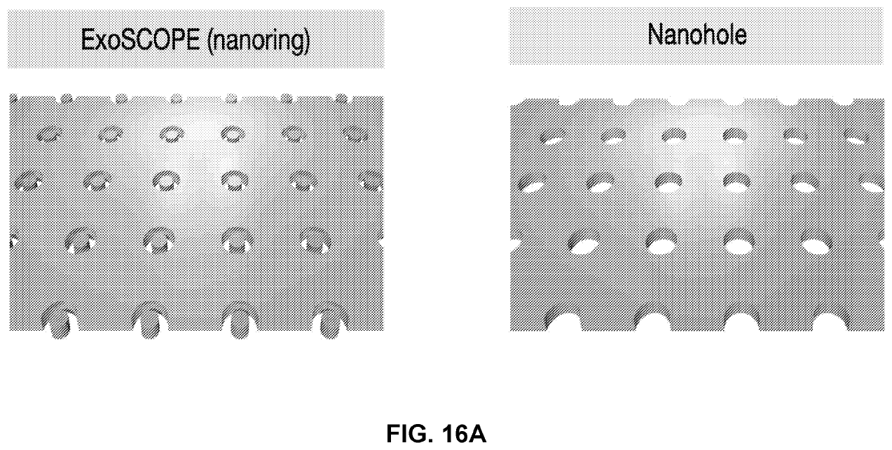

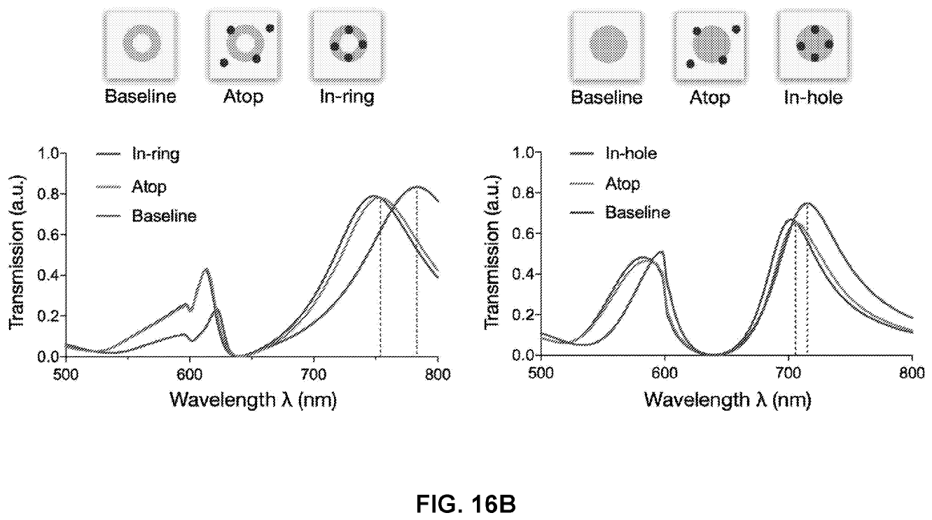

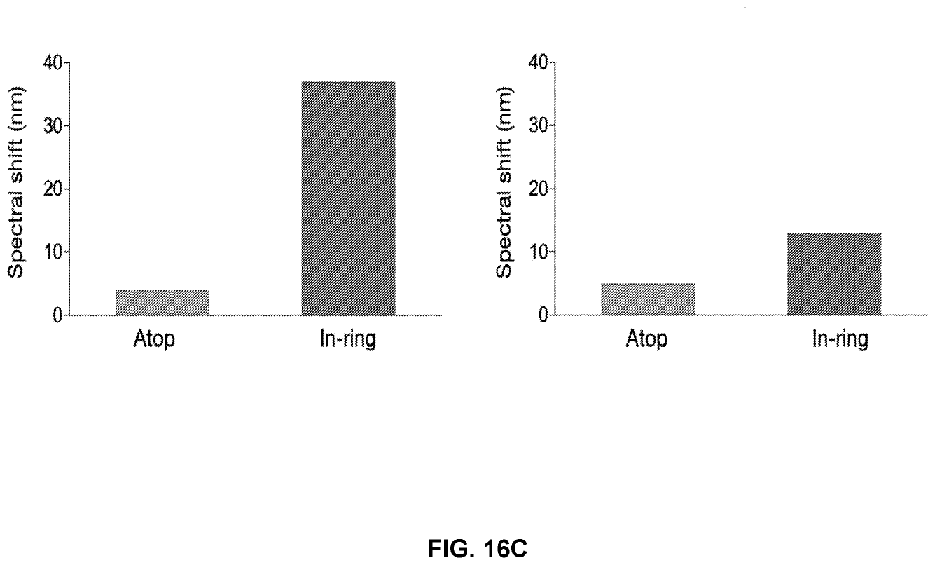

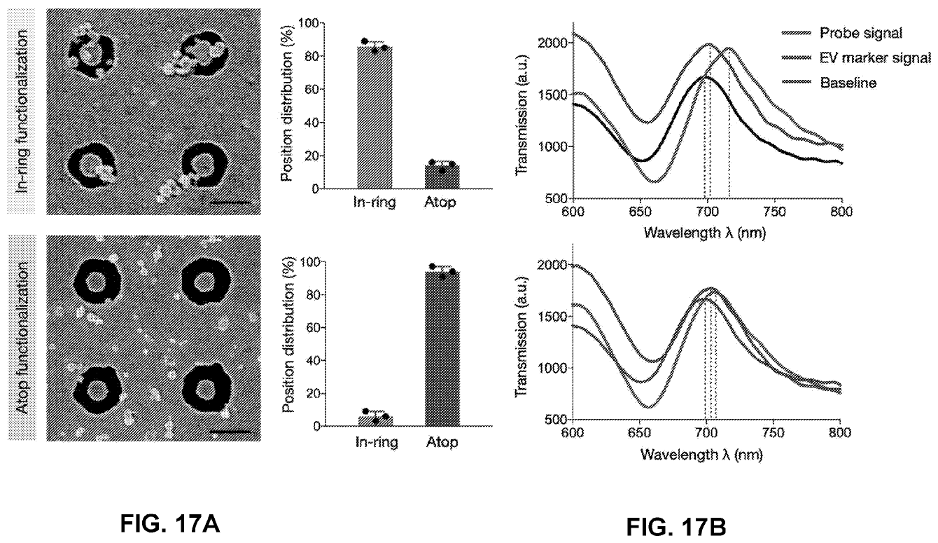

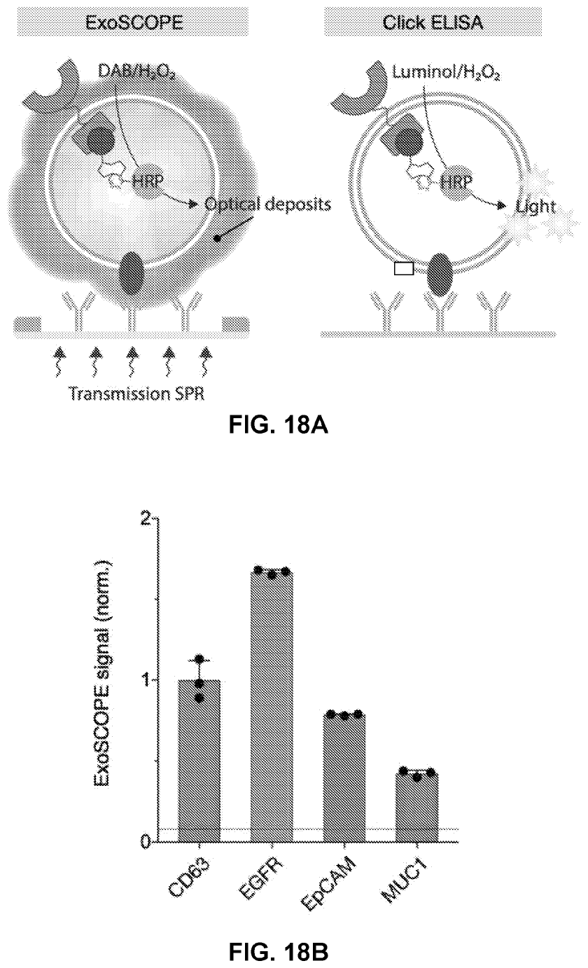

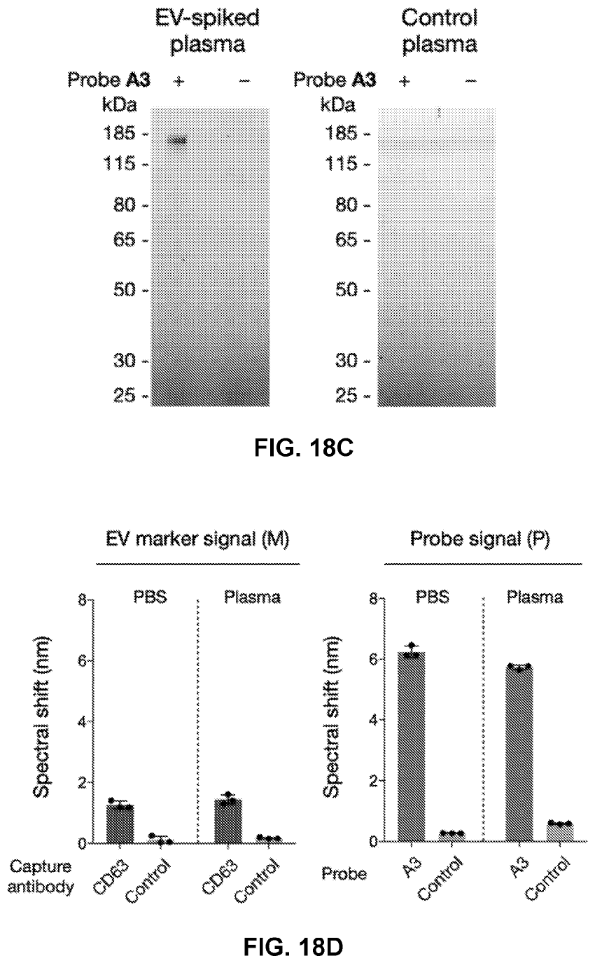

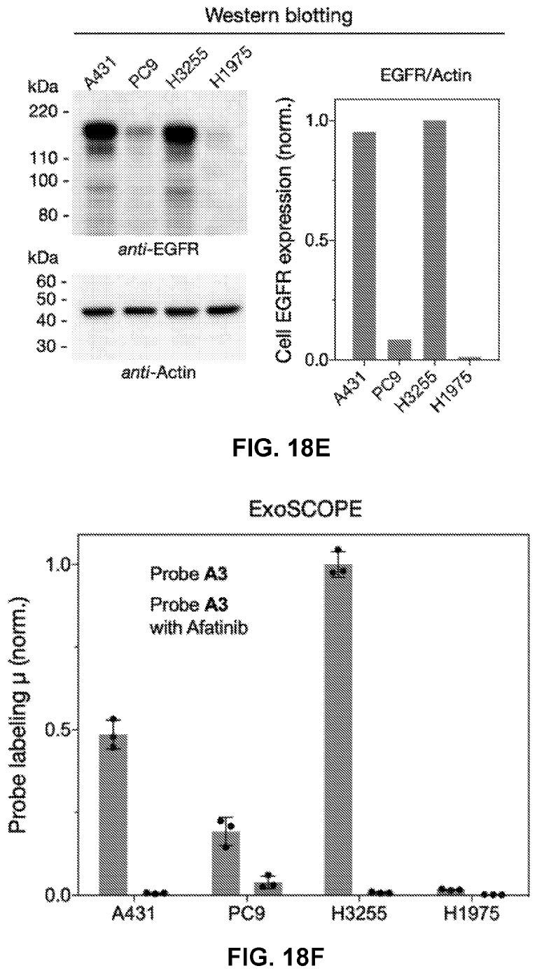

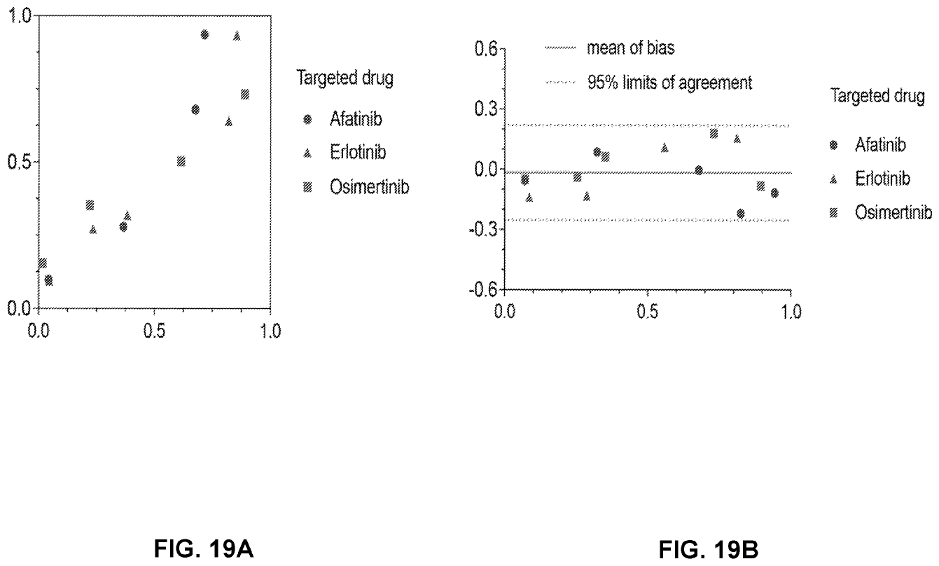

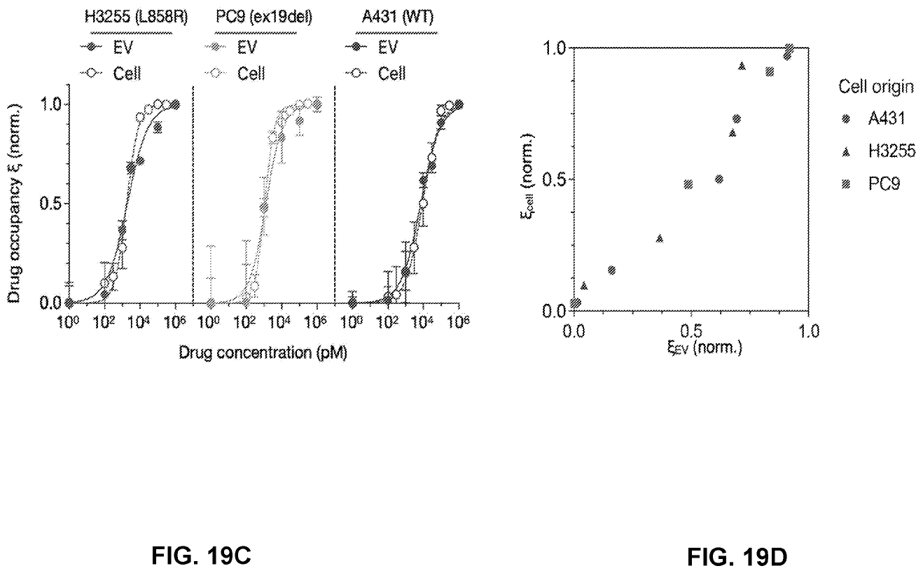

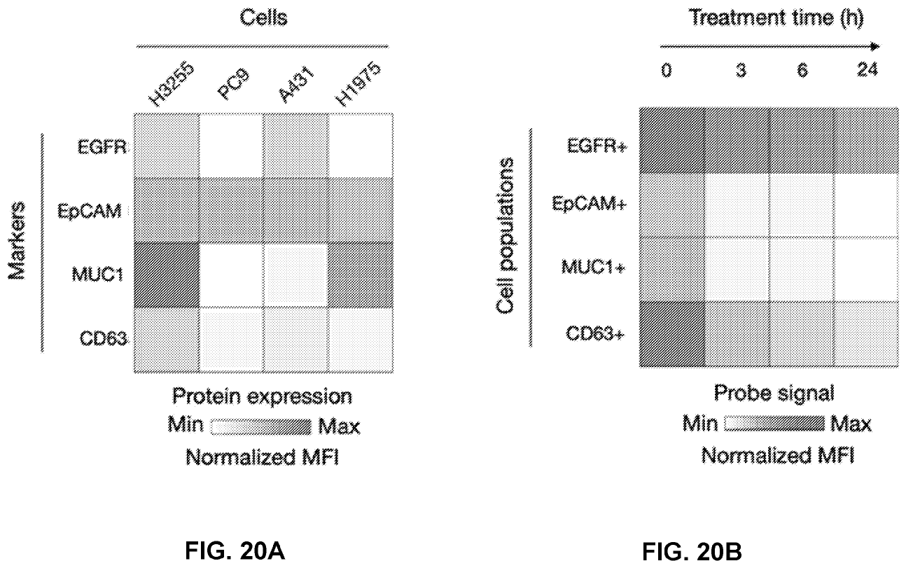

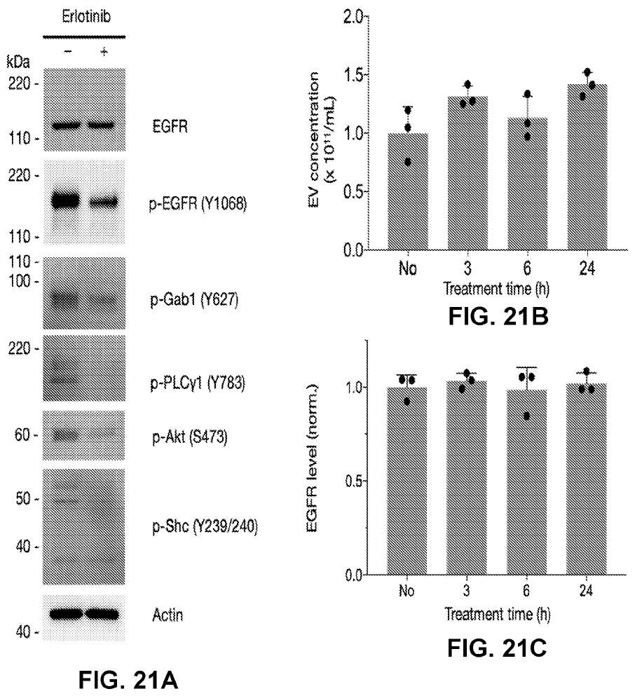

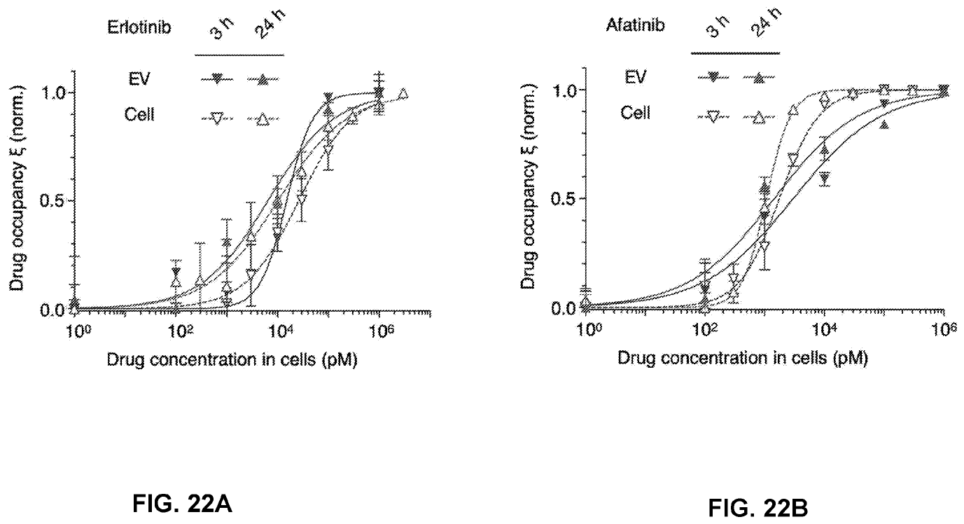

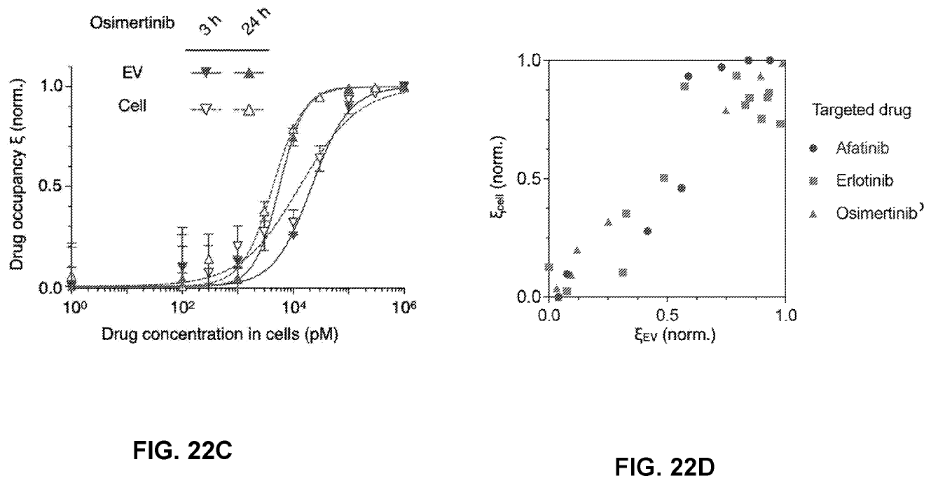

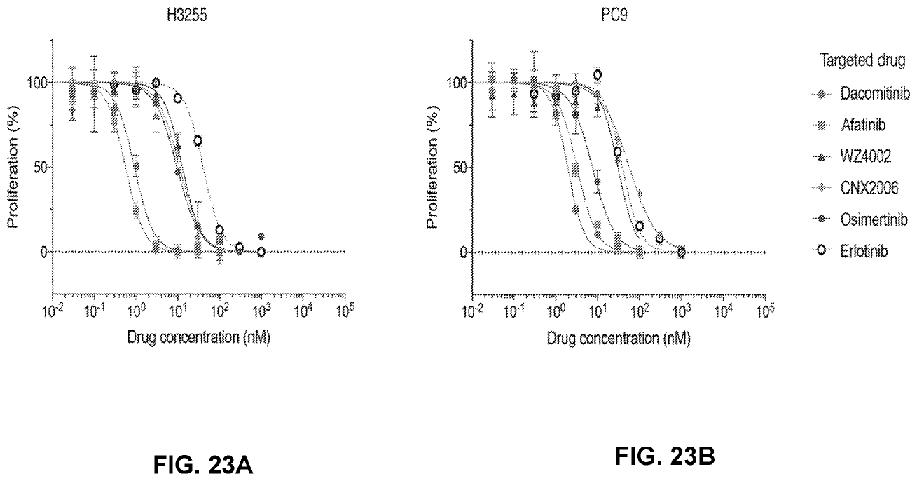

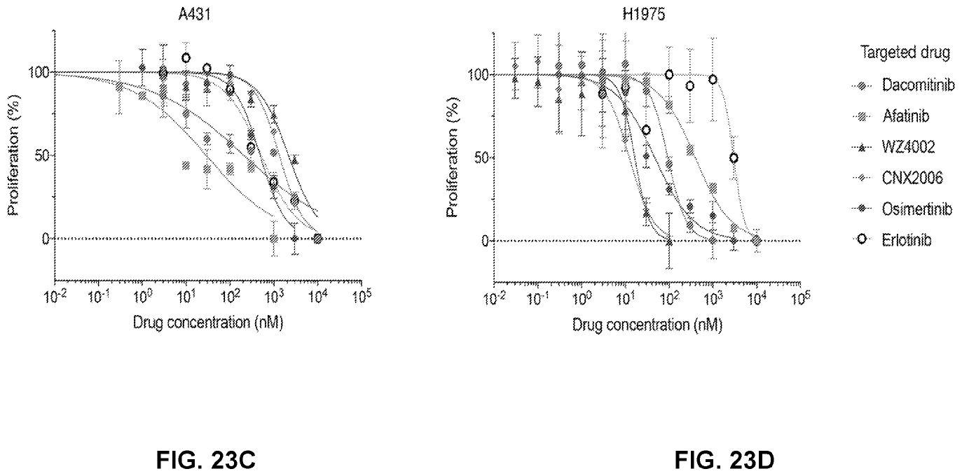

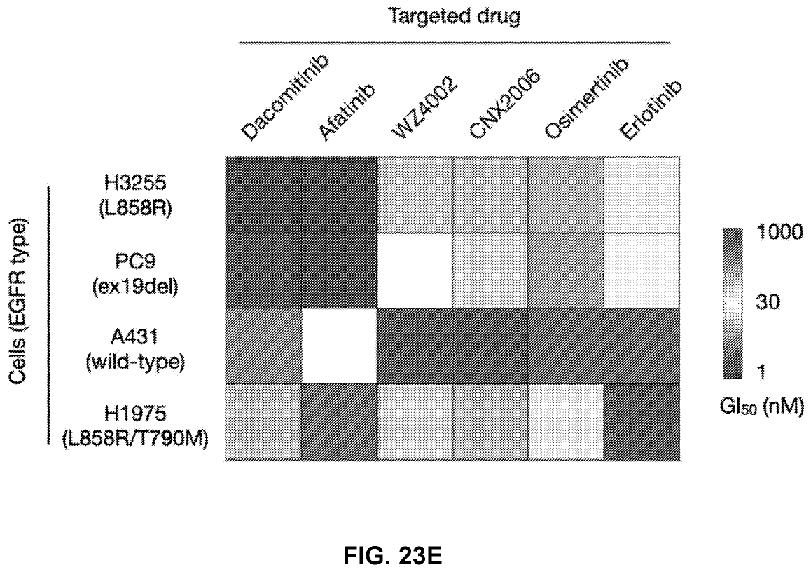

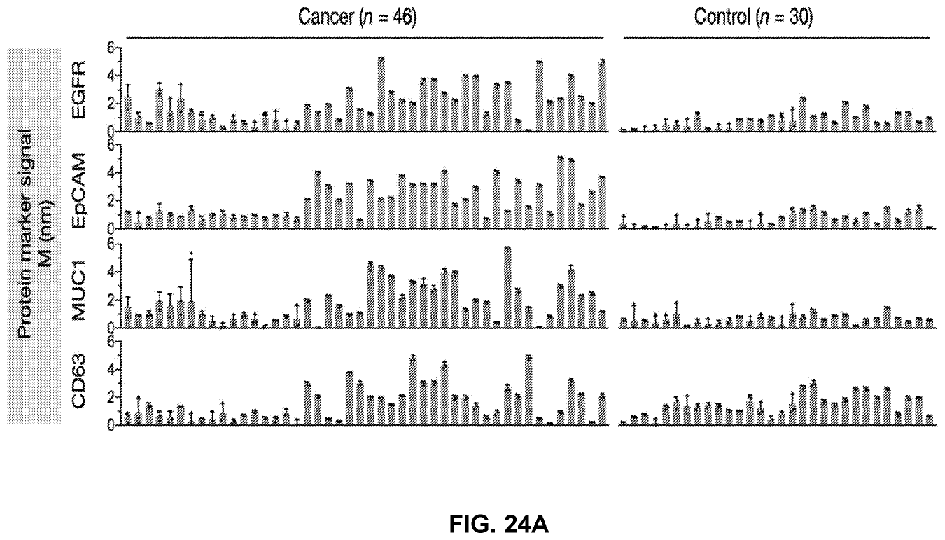

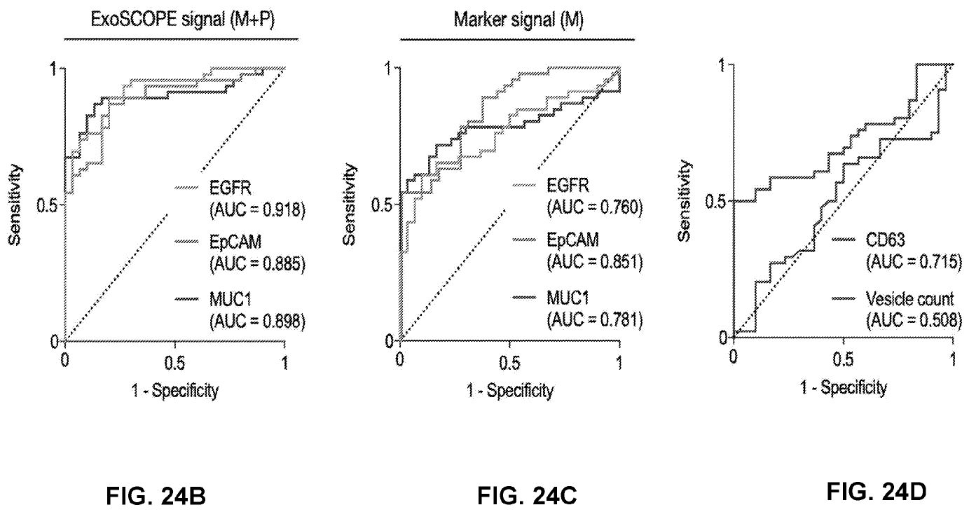

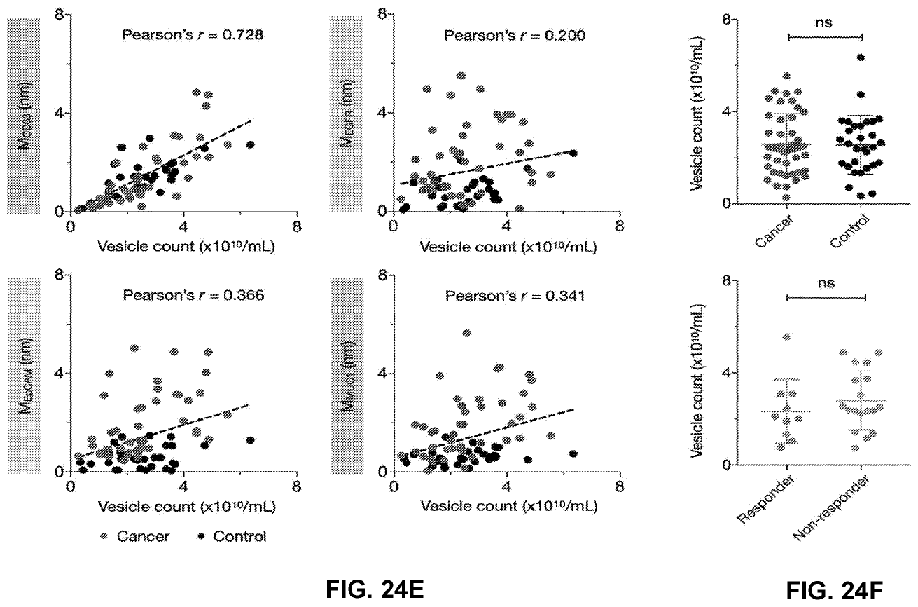

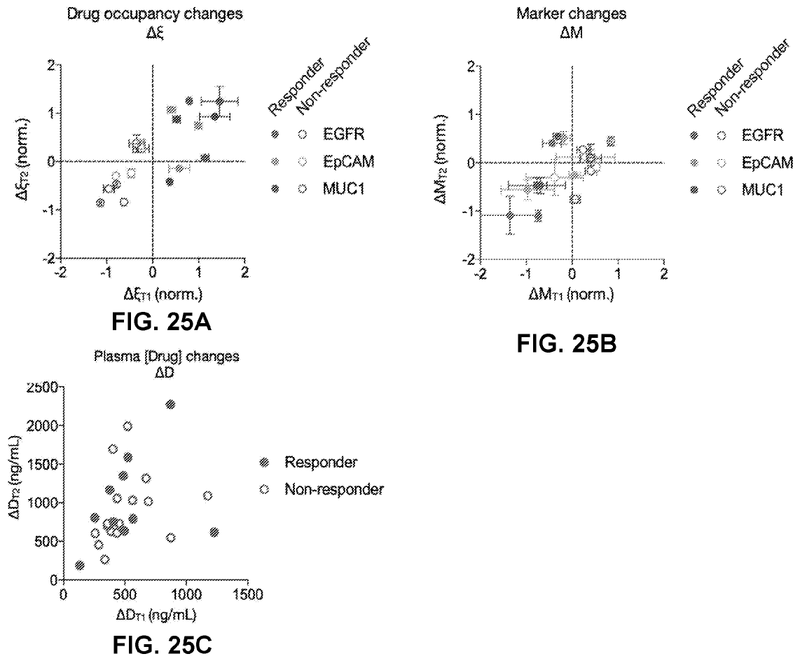

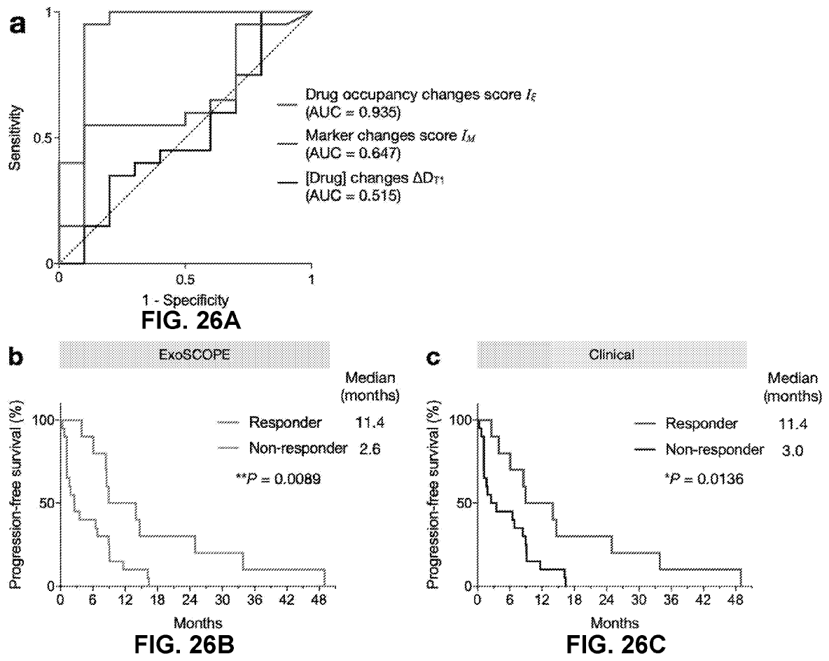

ExoSCOPE schematics. Drug-bound protein receptors are secreted through nanoscale extracellular vesicles (EVs). To measure EV drug occupancy and cellular drug effects, the ExoSCOPE platform utilizes competitive target labeling of EVs by bio-orthogonal click probes. These probes recruit enzymes (horseradish peroxidase) to achieve in situ deposition of insoluble optical products on labeled vesicles, thereby amplifying the probe-labeling signal. FIG. 1B. Spatial patterning of ExoSCOPE molecular reactions within plasmonic resonators. EVs are protein-typed and probe-amplified within plasmonic nanoring gaps, to exploit local electromagnetic hotspots for sensitive detection. Specifically, immuno-captured EVs that have a low drug occupancy are extensively labeled with competitive probes, whose bio-orthogonal handles enable in situ enzymatic amplification to locally deposit insoluble optical products. These optical deposits enhance plasmonic signals to generate a red shift in the transmitted light spectrum, thereby enabling multiparametric analysis of EV drug dynamics (i.e., protein composition and drug occupancy changes). FIG. 1C. Characterization of an ExoSCOPE click probe. Molecular docking simulation shows probe binding to the active site (red box) of EGFR kinase domain. The magnified view illustrates the probe in yellow and the parent drug (afatinib) in grey. Inset: transmission electron micrograph (TEM) of in situ vesicle labeling by the probe. TEM was performed after probe conjugation with gold nanoparticles (10 nm, indicated by arrows). FIG. 1D Plasmonic nanoring resonators. Left: scanning electron micrographs (SEM) of periodic lattices of gold nanorings, fabricated on a glass substrate. Right: enhanced electromagnetic fields are simulated to locate within the nanoring gaps (top), according to the measured cross-sectional dimensions of the nanorings (bottom). FIG. 1E. Real-time monitoring of targeted therapy in lung cancer patients. ExoSCOPE was applied to evaluate drug dynamics in cancer-associated EVs, directly in blood samples. In comparison to conventional blood pharmacologic analysis (PK/PD), ExoSCOPE could effectively distinguish treatment outcome.

FIG. 2A-2E. Design and evaluation of click probes. FIG. 2A Structures of the synthesized click probes. Using the parent drug (afatinib), we designed and prepared bio-orthogonal probes A1, A2 and A3. The probe handles and linkers are respectively colored; their corresponding reporters can be found in FIG. 12. FIG. 2B Anti-proliferation activities of the click probes. Lung cancer cells (H3255) were incubated with increasing concentrations of the probes for 72 hours. As compared to A2, probe A3 showed improved lipophilicity (calculated distribution coefficient at pH 7.4, cLogD) and functional activity (GI₅₀). FIG. 2C Live-cell labeling. Left: H3255 cells were treated with the probes (100 nM), with (+) or without (−) competitive afatinib (1 μM), and analyzed by in-gel fluorescence after click reactions with reporter dyes. Probe A3 not only showed afatinib-competitive and specific labeling of EGFR targets (matched molecular weight ˜170 kDa), but also demonstrated the best signal-to-noise ratio. Right: Western blotting analysis confirmed equal loading of EGFR in the cell lysates. FIG. 2D Real-time binding kinetics of probe A3 with different EGFR mutant proteins. We immobilized and incubated probe A3 with cell lysates containing different EGFR mutant proteins. All cell lysates were prepared in the absence or presence of afatinib treatment. Molecular binding was monitored through bio-layer interferometry. High binding potentials (Bₘₐₓ/K_{d}) were observed in cell lysates bearing L858R and ex19del EGFR mutations, consistent with the reported mutant selectivity of the parent drug afatinib.

FIG. 2E Drug-target engagement in cells. Competitive labeling of H3255 cells with various concentrations of afatinib showed drug dose-dependent decrease in probe A3 labeling. The results were independently validated by click ELISA, in-gel fluorescence and flow cytometry, with IC₅₀=1.4, 1.5, and 1.6 nM respectively. All measurements were performed in triplicate and the data are presented as mean±s.d. in b and e.

FIG. 3A-3F. Multiparametric analysis of EV drug occupancy. FIG. 3A Molecular characterization of A3 labeling in EVs. Left: SEM of EVs immuno-captured on a microbead through anti-CD63 antibody. Scale bar, 500 nm. Right: flow cytometry analysis of bead-bound EVs, after in situ probe labeling (100 nM) and click reaction with Cy5 reporter. FSC, forward scatter. FIG. 3B Probe amplification within plasmonic nanoring resonators. Left: finite-difference time-domain simulations. Enhanced electromagnetic fields are confined within the nanoring gap (in-ring). The in-ring assay exploits these plasmonic hotspots for sensitive, size-matched detection of EVs (indicated as blue circles). Scale bar: 100 nm. Right: experimental validation. EVs were immuno-captured and probe-amplified within the nanoring gaps. The corresponding marker and probe signals were measured. SEM analysis confirmed that molecular reactions were spatially localized. As compared to the atop configuration, the in-ring detection showed superior performance. FIG. 3C Detection sensitivity of the ExoSCOPE in-ring assay. The limit of detection was determined by titrating a known amount of EVs and measuring their A3 probe labeling signal through anti-CD63 vesicle capture. FIG. 3D Quantification of EGFR expression in EVs. Using EVs derived from various cell lines with known levels of EGFR expression, we measured the ExoSCOPE probe labeling index (μ) to evaluate the average probe density per sensor-bound vesicle. The measurements correlated well with ELISA analysis of vesicular EGFR expression. FIG. 3E Drug occupancy in EVs. We incubated EVs with increasing concentration of EGFR inhibitors (covalent: afatinib, osimertinib; and non-covalent: erlotinib) and employed the ExoSCOPE to measure relative EV drug occupancy (ξ_{EV}). A good agreement was observed between ξ_{EV} and independent ξ_{cell} analysis. FIG. 3F Mutant sensitivity of EV drug occupancy. By varying the doses of afatinib, EVs with EGFR mutant proteins demonstrated a lower drug occupancy-based IC₅₀ than those with wild-type proteins. All measurements were performed in triplicate and the data are presented as mean±s.d. in b-f.

FIG. 4A-4F. Multiplexed ExoSCOPE for longitudinal drug analysis. FIG. 4A Schematics of the multiplexed ExoSCOPE analysis. To evaluate molecular heterogeneity of targeted therapy, we used different antibodies to selectively profile EV subpopulations and their respective drug occupancies. For cellular analysis, we used the same marker panels for flow cytometry-based measurements. FIG. 4B Time-dependent drug occupancy in EV and cell subpopulations. We treated a heterogeneous cell mixture with targeted treatment (erlotinib, 1 μM) and measured drug occupancy changes in EVs and cells. FIG. 4C Longitudinal analysis with different targeted drugs. We incubated cells with respective treatments (i.e., different drugs at different concentrations) and measured time- and dose-dependent changes of drug occupancy in EVs. FIG. 4D Bland-Altman analysis. A good correlation was observed between ξ_{EV} and the corresponding ξcell, across multiple drugs and treatment conditions. FIG. 4E Comparison of EV drug occupancy and cellular drug potency. Lung cancer cells (H3255) were treated with six EGFR inhibitors (10 nM for 3 hours) to measure the resultant ξ_{EV}. When compared against their respective cellular potency (GI₅₀), more potent drugs with a low GI₅₀ (<1 nM) demonstrated a high ξ_{EV} (>0.6). FIG. 4F Correlation of ξ_{EV} and GI₅₀. We expanded the above study to other cell lines. Two sets of linear relationships were observed, which could distinguish sensitive (H3255, PC9) vs. resistant (A431, H1975) cell lines. All measurements were performed in triplicate and the data are presented as mean±s.d. in FIGS. 4B-4F.

FIG. 5A-5E. Clinical profiling of lung cancer patients. FIG. 5A Multiplexed ExoSCOPE analysis of clinical plasma samples. Samples were obtained from lung cancer patients (n=46) and healthy controls (n=30). FIG. 5B Receiver operating characteristic curves of the ExoSCOPE analyses. The composite cancer signature, based on EGFR, EpCAM and MUC1, showed a high accuracy to diagnose lung cancer. FIG. 5C Longitudinal monitoring of targeted therapy. Plasma samples were collected from lung cancer patients at various time points: To, before treatment (baseline); T₁, 24 hours (day−1) after erlotinib treatment initiation; T2, 192 hours (day−8) after treatment initiation. Responder and non-responder status was clinically determined at the end of the treatment (day−21). Multiplexed ExoSCOPE was performed to measure changes in EV drug occupancy (Δξ) as well as changes in EV protein marker composition (ΔM). Total drug concentration in plasma (ΔD) was independently determined through conventional blood pharmacologic analysis (PK/PD). FIG. 5D Multiplexed ExoSCOPE for early time point (T₁) assessments. Across different EV subpopulations, we measured respective longitudinal changes (T₁ with respect to T₀) in EV drug occupancy (Δξ_{T1}) and EV protein marker (ΔM_{T1}), and used the data to construct regression models for scoring drug occupancy changes (I_{ξ}) and marker composition changes (I_{M}), respectively. Corresponding changes in plasma drug concentration is denoted ΔD_{T1}. FIG. 5E ExoSCOPE differentiation of treatment outcome. EV drug analysis (I_{ξ}) at early time point (T₁) could effectively distinguish responders from non-responders, while the other metrics could not (n=30 patients). (****P<0.0001, ns: not significant; Student's t-test). All measurements were performed in triplicate and the data are presented as mean±s.d. in FIGS. 5A, 5C and 5E.

FIG. 6A-6E. Multimodal characterization of vesicles derived from lung cancer cells. FIG. 6A Transmission electron micrograph (TEM) of EVs isolated from lung cancer cells (H3255). Inset shows a magnified view of a single vesicle. FIG. 6B Western blotting analysis of EV and cell lysates (H3255). The lysates, loaded at equal protein amount, were probed for receptor target (EGFR), loading control (GAPDH), EV markers (CD63, LAMP-1, Alix, HSP90, Flotillin 1, TSG101), and cellular-specific contaminant markers (GM130, Calnexin). The analysis showed the presence of EGFR in EVs, an enrichment of EV markers and an absence of cellular contamination in the vesicle preparation. FIG. 6C Unimodal size distribution of EVs derived from H3255 cell line, as determined by nanoparticle tracking analysis. The mean diameter was ˜100 nm. FIG. 6D Direct EV treatment. EVs isolated from untreated lung cancer cells (H3255) were incubated with probe A3 (100 nM) in the absence (−) or presence (+) of drug (1 μM). In-gel fluorescence analysis of the vesicle lysates, after click reaction with tetrazine-TMR dye, confirmed probe A3's ability for in situ labeling of vesicular EGFR and that the labeling is specific and afatinib-competitive (top). Western blotting analysis of EGFR and CD63 showed equal loading of the EV lysates (bottom). FIG. 6E Cell treatment. Lung cancer cells (H3255) were incubated with (+) or without (−) probe A3 (100 nM). After removal of the unbound probes, EVs were isolated from the cell culture. In-gel fluorescence and western blotting analysis showed the presence of probe-bound receptors in EVs.

FIG. 7A-7B. ExoSCOPE workflow and analysis. FIG. 7A Step-by-step workflow. The ExoSCOPE leverages competitive target labeling by bio-orthogonal click probes to measure EV drug changes. To enable multiparametric measurements, we perform a series of operations, namely antibody functionalization on the sensor (baseline measurement), marker-induced EV binding (signal M), and probe-induced amplification (signal P), and measure the associated changes in transmitted light spectra for the corresponding molecular signals. Specifically, to measure EV binding (marker-induced, M), vesicles are immuno-captured onto the functionalized sensors. To measure probe labeling (of sensor-bound vesicles, P), we use the click probes' bio-orthogonal handle (trans-cyclooctene, TCO) to recruit tetrazine-conjugated horseradish peroxidase (HRP), which catalyzes in situ, enzymatic conversion of the soluble substrate (3,3′-diaminobenzidine, DAB) to form local, insoluble polymeric products on the labeled vesicles. These insoluble deposits act as optical products to amplify the plasmonic signals. FIG. 7B ExoSCOPE drug occupancy analysis. EV drug occupancy is assessed through competitive probe labeling and enzymatic probe amplification. In comparison to vesicles with a high drug occupancy, EVs with a low drug occupancy are more extensively probe-labeled and amplified. The formation of localized, high-density optical deposits in these vesicles results in a red shift in the transmitted light spectrum, leading to an increased P signal. To account for differences in vesicle counts and composition across samples, we use the multiparametric measurements to perform analysis. Specifically, the ExoSCOPE measures marker-induced EV binding signal (M, promotional to the number of sensor-bound, antibody-captured vesicles) and probe-induced amplification signal (P, proportional to the total number of probes found within the captured vesicles). We use these parameters to define the probe labeling index (=P/M); this normalization reflects an average probe density per antibody-captured vesicle. To quantify relative drug-target engagement in EVs, we define the drug occupancy index (ξ=1−μ/μ⁰, where μ⁰ refers to that of a matched control not treated with drug and thus serves as a positive control for probe labeling).

FIGS. 8A and 8B. ExoSCOPE spatial patterning through differential material functionalization. Schematics for spatial patterning of molecular reactions. FIG. 8A shows the process of in-ring functionalization, where the glass substrate of ExoSCOPE is first coated with APTES, and the capture antibodies are covalently attached to APTES using glutaraldehyde. FIG. 8B shows atop functionalization, where the gold layer is coated with carboxylated (HS-PEG-COOH) and methylated (HS-PEG-Me) thiol-PEG, and the capture antibodies are then coupled to the carboxylate group via EDC/NHC chemistry. Subsequently, both sensors can be applied through identical steps of EV capture and probe amplification, and the transmitted spectral shifts are measured accordingly.

FIG. 9A-9D. Optimization of the ExoSCOPE sensor. FIG. 9A Optimization of nanoring dimensions. We performed numerical simulations to optimize the nanoplasmonics for EV detection within the nanoring gap (left). The optimal condition is highlighted. Experimental verification showed a good correlation to the simulated results (right). FIG. 9B Photograph of the developed ExoSCOPE sensor. The sensor array consists of 3×7 sensing elements, patterned on a glass substrate (left). Each element comprises periodic lattices of gold nanorings. Large area scanning electron micrograph (SEM) showed uniformly fabricated nanorings (right). FIG. 9C Transmitted light spectra of the ExoSCOPE sensor. Increases in the refractive index induced a red shift in the transmission peak, towards the longer wavelength. FIG. 9D A linear correlation could be obtained between the changes in refractive index (RI) and the shifts in the transmitted peak wavelength. a.u., arbitrary unit.

FIG. 10A-10C. Evaluation of sensor variability. FIG. 10A Variability in nanoring fabrication. The nanoring resonators were patterned in a gold film to achieve the following optimized dimensions: 50 nm (thickness), 150 nm (inner ring diameter) and 350 (outer ring diameter). The results showed uniform fabrication and consistent optical performance across sensors. All characterization measurements were performed through SEM and AFM analysis. FIG. 10B Antibody functionalization. Across different functionalization experiments and antibodies used, antibody attachment coverage remained consistent. FIG. 10C Capturing efficiency with different antibodies and different EVs. EVs derived from different cell culture (A431 and H3255) could be effectively captured by anti-CD63 antibody. Negative control (IgG control antibody) showed negligible EV binding. All measurements were performed in triplicate and the data in b and c are presented as mean±s.d. (ns: not significant; Student's t-test).

FIG. 11A illustrates synthesis of click probes. The synthesis began with a commercially available intermediate 1, which is commonly used for afatinib preparation. Firstly, substitution reaction was performed on 1 to incorporate a 3-carbon linker containing a Boc-protected amine. Next, the nitro group on 2 was reduced to amine and then functionalized with the Michael acceptor as afatinib. Finally, the Boc protection group on 3 was removed and the resulting amino group was either converted to azide (A1) or coupled with different TCO building blocks to yield the click probes (A2, A3) accordingly. The purity of click probes was confirmed with high performance liquid chromatography (HPLC) and high resolution mass spectrometry (HRMS). FIG. 11B. See Example 8 for synthesis details and NMR characterization.

FIG. 12A-12C show click chemistry of probes and reporters. FIG. 12A illustrates ligating Azide (on probe A1) to dibenzocyclooctyne (DBCO) via strain-promoted alkyne-azide cycloaddition. FIG. 12B and FIG. 12C illustrate directly ligating TCO (A2, A3) to tetrazine through inverse electron-demand Diels-Alder reaction. All structures of the probes and their paired fluorescence reporters, DBCO-TMR, Tetrazine-TMR and Tetrazine-Cy5, as well as biotinylated reporter Tetrazine-Biotin, are illustrated.

FIG. 13A-13D. Target labeling by different click probes. Results of lysate labeling and live-cell labeling were compared. Click probes (100 nM) were used to label H3255 cells and revealed by in-gel fluorescence after click reaction with respective TMR dye reporter (FIG. 13A). Click ELISA analysis was performed independently to quantify the EGFR labeling by probe A2 and A3 through the live-cell incubation (FIG. 13B). ELISA analysis was performed through protein immuno-capture with anti-EGFR antibody after click reaction with Tetrazine-Biotin reporter. FIG. 13C shows results of Coomassie stain of the gels in FIG. 13 A, which showed equal loading of the lysates. The live-cell labeling samples were further analyzed through western blotting for EGFR and Actin and results were shown in FIG. 13D.

FIG. 14A-14D. Performance evaluation of probe A3 in live cells. FIG. 14A Optimization of probe incubation time. Probe A3 (100 nM) was used to label H3255 cells at increasing incubation time. The probe labeling was revealed by in-gel fluorescence after click reaction with Tetrazine-TMR dye (left) or coomassie stain (right). FIG. 14B Time-dependent labeling efficiency. Analysis of A3-labeled EGFR bands revealed time-dependent labeling, which reached 90% efficiency after 15 min incubation and maxima at 1 hr. FIG. 14C Competitive labeling with afatinib. H3255 cells were drug-treated with varying concentration of afatinib and labeled with probe A3 (100 nM). In-gel fluorescence revealed drug dose-dependent decrease in probe A3 labeling. FIG. 14D Flow cytometry analysis of the afatinib-treated cells confirmed the competitive A3 labeling. Analysis was performed upon A3 labeling and reaction with Tetrazine-Cy5.

FIG. 15. Characterization of EVs and optical deposits. SEM images of control beads, beads with EVs and that after deposition of insoluble, optical products. For enzymatic deposition of optical products, probe-labeled EVs were captured onto antibody-functionalized polystyrene beads (through anti-CD63 capture). The bead-bound vesicles were then incubated with enzyme (HRP) and substrate (DAB) to catalyze the localized deposition of insoluble products. Distributional analysis showed an increase in mean particle size after the formation of insoluble deposits.

FIG. 16A-16C. Theoretical comparison of nanoring and nanohole structures. FIG. 16A Schematics of the nanoring and nanohole structures. Both nanostructures have identical periodicity, thickness, and outer diameter. FIG. 16B Simulated transmission spectra when EVs (red dots) are captured at different locations on the nanoring or nanohole structures. With an equal amount of EV binding, the nanoring (in-ring) configuration experiences the largest transmission spectral shift. FIG. 16C The nanoring detection shows enhanced performance over the nanohole assays, especially when EVs are captured in the nanoring gap (in-ring), where the strongest electromagnetic field is located. All spectral shifts are determined from the transmitted peak wavelengths, relative to respective baseline measurements.

FIG. 17A-17B. Experimental comparison of nanoring and nanohole structures. FIG. 17A SEM characterization on the spatial distribution of bound EVs. The sensors were treated with different functionalization conditions. EVs were immuno-captured and probe-amplified. For the in-ring functionalization, ˜85% of the bound targets were located within the nanoring gap (in-ring). For the atop functionalization, ˜5% of the targets were captured in-ring and ˜95% were bound to the gold surface (atop). FIG. 17B Experimental sensorgrams showed the superior performance of the in-ring functionalization. The optical spectra were determined after antibody conjugation (baseline), EV capture (EV marker signal) and probe amplification (probe signal), respectively.

FIG. 18A-18F. Multiparametric ExoSCOPE detection of EGFR. FIG. 18A Schematics of the ExoSCOPE platform and click ELISA. In both assays, probe-labeled vesicles were antibody-captured onto the sensor surface, and incubated with HRP for enzymatic amplification. In the ExoSCOPE analysis, the bound HRP was used to convert soluble DAB substrate into insoluble deposits over labeled vesicles; this localized deposition of optical products results in an enhanced plasmonic signal (left). In the click ELISA, the bound HRP was used to generate chemiluminescence signal, through the conversion of Luminol substrate in solution (right). FIG. 18B ExoSCOPE analytical performance. To evaluate multiplexed ExoSCOPE analysis with a low number of cancer EVs, measurements were performed on 1.0×10⁴ cancer EVs (derived from H3255 lung cancer cell culture). Probe-labeled vesicles were captured through anti-CD63 antibodies or respective cancer-marker antibodies, before HRP enzymatic amplification for ExoSCOPE analysis. All ExoSCOPE signals were above the limit of detection, indicated as the dotted line (3× s.d. of the signal of a sample-matched isotype control). FIG. 18C Probe labeling in plasma. EVs (derived from H3255 culture) were spiked into control plasma. Both the spiked sample and control plasma were labeled with probe A3, reacted with dye reporter and imaged through in-gel fluorescence. Only in the spiked preparation, probe A3 demonstrated specific binding to molecular targets (EGFR, ˜170 kDa), with minimal cross-reactivity to other proteins. In the control plasma, negligible binding was observed by probe A3. FIG. 18D ExoSCOPE analysis of EVs spiked into plasma. EV marker signal (M) was measured through anti-CD63 capture antibody. Probe signal (P) was measure through A3 labeling and enzymatic amplification. Sample-matched control measurements were performed with IgG isotope control antibody. Plasma-spiked EV measurements demonstrated similar signals to that of pure EVs in PBS. FIG. 18E Western blotting analysis of EGFR expression in different cell lysates. EGFR expression levels were normalized to that of Actin. FIG. 18F ExoSCOPE analysis of EVs derived from different cell lines. EVs were treated without or with afatinib (1 μM) before A3 probe labeling and detection. All measurements were performed in triplicate, and the data are displayed as mean±s.d. in FIGS. 18C, 18D and 18F.

FIG. 19A-19C. Correlation of EV and cellular drug occupancy. Correlation of EV (ξ_{EV}) and cellular ξcell) drug occupancy by FIG. 19A Pearson analysis and FIG. 19B Bland-Altman analysis. The analyses demonstrate good agreement, across different types of targeted EGFR inhibitors (irreversible: afatinib and osimertinib, reversible: erlotinib), at varied drug doses. FIGS. 19C-19D EVs and parent cells showed similar dose-dependent ξ_{EV} and ξ_{cell} curves against afatinib competition. EVs and cells bearing EGFR mutants (H3255 with L858R mutation and PC9 with ex19del mutation) showed a lower IC₅₀ than those with wild-type (A431), consistent with the mutant selectivity of the parent drug afatinib. All measurements were performed in triplicate, and the data are displayed as mean in a, b and d, and as mean±s.d. in FIG. 19C.

FIG. 20A-20B. Flow cytometry analysis of cellular protein markers. FIG. 20A Expression levels of putative cancer markers (EGFR, EpCAM, MUC1) and EV marker (CD63) in different cancer cell lines. All measurements were normalized against that of IgG isotype control antibodies. FIG. 20B Probe signal changes during drug treatment. A heterogeneous cell mixture was treated with erlotinib (1 μM) or vehicle (DMSO). Samples were obtained from the mixture at various time points during drug treatment and labeled with probe A3 (100 nM). To examine the longitudinal samples, we performed antibody-based cell gating to identify various cell subpopulations, based on their cellular marker expressions, and measured their respective probe labeling signals. All measurements were performed in triplicate, and the data are displayed as mean. MFI, mean fluorescence intensity.

FIG. 21A-21C.Western blotting analysis of H3255 cells. The cells were treated with erlotinib (+, 1 μM) or vehicle (−, DMSO) for 6 hours. Receptor targets (EGFR and p-EGFR) and their downstream signaling proteins (p-Gab1, p-PLC71, p-Akt, p-Src) were quantified. FIG. 21B EV concentrations in H3255 cell culture treated with erlotinib (1 μM), as determined by NTA. FIG. 21C EGFR expression levels in the EV samples from FIG. 21B, as measured by ELISA and normalized against CD63 expression. All measurements were performed in triplicate, and the data are displayed as mean±s.d. in FIGS. 21B and 21C.

FIG. 22A-22D. Time-dependent changes in EV and cellular drug occupancy. Time-dependent changes in EV and cellular drug occupancy. (a-c) H3255 cells were treated with varied concentrations of FIG. 22A erlotinib, FIG. 22B afatinib or FIG. 22C osimertinib for 3 hours or 24 hours, respectively. After treatment, the cells and secreted EVs were analyzed separately to evaluate their respective dose-dependent drug occupancy ξ_{cell} and ξ_{EV}). FIG. 22D Pearson analysis showed a good correlation of EV and ξ_{cell} at different doses and treatment durations, across different targeted drugs. All measurements were performed in triplicate, and the data are displayed as mean±s.d. in FIGS. 22A-22C.

FIG. 23A-23D. Cellular growth inhibition by targeted inhibitors. FIGS. 23A-23D shows proliferation inhibition of EGFR inhibitors on cancer cell lines known to express EGFR mutants L858R (H3255), ex19del (PC9), wild-type (A431) and L858R/T790M (H1975). Cells were treated with six EGFR inhibitors (afatinib, erlotinib, osimertinib, dacomitinib, WZ4002, CNX2006) for three days. Dose-dependent growth inhibition was determined by MTS assays. All measurements were performed in triplicate, and the data are displayed as mean±s.d. FIG. 23E Summary of GI₅₀ (nM), indicating that H3255 and PC9 cells are more sensitive to these drugs.

FIG. 24A-24F. Other EV analyses to diagnose lung cancer. FIG. 24A EV protein markers (M) were measured in lung cancer patients (n=46) and healthy controls (n=30). Putative cancer markers (EGFR, EpCAM and MUC1) as well as EV marker (CD63) were quantified in individual samples. FIGS. 24B, 24C ROC curve analysis of the ExoSCOPE signals as well as marker signals. FIG. 24D ROC curve analysis of CD63 marker signals and vesicle counts showed poor diagnostic accuracy. FIG. 24E Correlation of ExoSCOPE marker signals with vesicle counts. Only CD63 analysis showed a good agreement to vesicle counts. Poor correlations were observed between respective cancer-marker signals and vesicle counts. FIG. 24F Vesicle count distribution in clinical samples. No significant difference was observed between cancer vs. control samples, nor responder vs. non-responder samples. All measurements were performed in triplicate, and the data are displayed as mean±s.d. in FIGS. 24A and 24F. (ns: not significant; Student's t-test).

FIG. 25A-25C. Longitudinal treatment monitoring by ExoSCOPE and conventional blood analysis. Scatter plots of longitudinal ExoSCOPE changes in FIG. 25A EV drug occupancy (Δξ) and FIG. 25B protein marker (ΔM). Plasma samples were obtained from lung cancer patients undergoing targeted erlotinib treatment, at various time points (To: before treatment, baseline; T₁, 24 hours since treatment initiation; T₂, 192 hours since treatment initiation). All time-stamped changes were made with respect to the baseline (T₀) measurements. Only the EV drug occupancy changes (Δξ) could differentiate the clinical responders from non-responders. FIG. 25C Plasma drug concentration changes (ΔD) at both time points could not differentiate the responders from non-responders.

FIG. 26A-26C. Evaluation of ExoSCOPE treatment monitoring. FIG. 26A ROC curve analysis of the composite scores for treatment monitoring: drug occupancy changes score (I_{ξ}), marker changes score (I_{M}) and plasma drug concentration (ΔD_{T1}). All measurements were performed at T₁ (24 hours after treatment initiation) with respect to T₀ (baseline, before treatment). The composite scores were computed through a cross-trained regression model based on linear combination of analyses. I_{ξ} showed the highest accuracy to predict patient response at the early time point T₁. (b-c) Kaplan-Meier analysis of progression-free survival in lung cancer patients. Patient response was classified based on FIG. 26B ExoSCOPE drug occupancy changes (I_{ξ}>0.50) at 24 hours after treatment initiation, or FIG. 26C imaging assessment using RECIST criteria at the end of treatment (21 days post treatment).

### DETAILED DESCRIPTION

### Overview

The present disclosure relates to a technology, termed extracellular vesicle analysis of small-molecule chemical occupancy and protein engagement (ExoSCOPE), which utilizes bio-orthogonal probe amplification and spatial patterning of molecular reactions within matched plasmonic nanoresonators for in situ analysis of EV drug dynamics. The technology is sensitive and informative. It detects delicate changes of drug binding with mutant proteins, provides multiparametric evaluation-drug occupancy and protein composition in molecular subpopulations of extracellular vesicles, and reveals real-time cellular changes of drug engagement and potency across different targeted drugs. When applied for clinical cancer monitoring, through scant patient blood, the ExoSCOPE not only accurately classified disease status, but also rapidly distinguished targeted treatment outcomes, e.g., within 24 hours after treatment initiation.

The present disclosure provides an analytical platform to leverage circulating extracellular vesicles for activity-based monitoring of tumor-specific drug-target interactions, directly in native blood samples. The technology, termed extracellular vesicle analysis of drug occupancy and protein engagement (ExoSCOPE), utilizes bio-orthogonal probe amplification and spatial patterning of molecular reactions within matched plasmonic nanoring resonators, to achieve in situ analysis of EV drug dynamics. The technology is sensitive and informative. It detects delicate changes of drug binding with mutant proteins, provides multiparametric evaluation—drug occupancy and protein composition—in molecular subpopulations of extracellular vesicles, and reveals real-time cellular changes of drug engagement and potency, across different targeted drugs. When applied for clinical cancer monitoring, through scant patient blood, the ExoSCOPE not only accurately classified disease status, but also rapidly distinguished targeted treatment outcomes, e.g., within 24 hours after treatment initiation.

In some aspects, the methods and compositions disclosed herein leverage bio-orthogonal probe amplification, spatial patterning of molecular reactions within matched plasmonic nanoring resonators and in situ enzymatic conversion of optical product for localized signal amplification for signal amplification. The platform thus enables 1) high sensitivity; The platform showed a limit of detection (LOD) of ˜1,000 probe-labeled extracellular vesicles, which is 104-fold better than that of ELISA-based assay. 2) measurement of time-dependent drug dynamics in distinct subpopulations of secreted vesicles. 3) examine serial blood samples of cancer patients undergoing targeted therapy and not only accurately classify disease status, but also effectively distinguish treatment outcomes.

The technology also employs spatially-optimized plasmonic nanoresonators (e.g., nanoring resonators) and in situ enzymatic conversion of localized optical product for signal amplification and molecular co-localization to enable highly sensitive, multiplexed population analysis.

Additional benefits provided by the technology disclosed herein include but are not limited to the following.

The ExoSCOPE classification based on vesicular drug dynamics was accurate and correlated well with clinical patient survival data, indicating the effectiveness of the technology for early monitoring of targeted treatment outcomes.

The methods and compositions disclosed herein enable inexpensive, direct, non-invasive and quantitative detection of lung cancers: Current clinical evaluation of targeted cancer therapies in solid tumors relies primarily on tumor volumetric imaging, which is delayed and insensitive to drug molecular interactions and mechanisms. Therefore, we develop a dedicated analytical platform to leverage circulating extracellular vesicles for activity-based monitoring of tumor-specific drug-target interactions, directly in native blood samples. Through multiplexed analysis of extracellular vesicle subpopulations, the invention could accurately detected cancer patients, and further revealed drug occupancy signatures to distinguish treatment efficacy.

The methods and compositions disclosed herein enable early monitoring of targeted therapy outcomes: Clinically, responder and non-responder status was determined at the end of the treatment (day−21) by tumor volumetric imaging. Through multiplexed analysis on time-dependent changes in EV drug occupancy (Δξ), this invention could effectively distinguish responders from non-responders (P<0.0005) undergoing targeted treatment of EGFR inhibitor. The difference could be observed as early as in 24 hours after treatment initiation, using only 5 μL of native plasma samples

Various features related to the ExoSCOPE technology are set forth below and further explained in detail.

### Terminology

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject.

As used in herein, the singular forms “a”, “an” and “the” include plural referents unless the content clearly dictates otherwise. Thus, for example, reference to “an antibody” optionally includes a combination of two or more such molecules, and the like.

The term “about” as used herein refers to the usual error range for the respective value readily known to the skilled person in this technical field, for example ±20%, ±10%, or ±5%, are within the intended meaning of the recited value.

The term “biomarker” as used herein is understood to be an agent or entity whose presence or level correlates with an event of interest. The biomarker may be a cell, a protein, nucleic acid, peptide, glycopeptide, an extracellular vesicle, or combinations thereof. For example, the biomarker is an EGFR protein whose presence or level indicates whether a subject suffers from, or is at risk of developing lung cancer.

The term “cancer marker” refers to a biomarker that is preferentially expressed in cancer than a normal tissue.

The term “subject” means any animal, including any vertebrate or mammal, and, in particular, a human, and can also be referred to, e.g., as an individual or patient.

The term “antibody” includes, but is not limited to, synthetic antibodies, monoclonal antibodies, recombinantly produced antibodies, multispecific antibodies (including bi-specific antibodies), human antibodies, humanized antibodies, chimeric antibodies, single-chain Fvs (scFv), Fab fragments, F(ab′) fragments, disulfide-linked Fvs (sdFv) (including bi-specific sdFvs), and anti-idiotypic (anti-Id) antibodies, and epitope-binding fragments of any of the above. The antibodies provided herein may be monospecific, bispecific, trispecific or of greater multi-specificity. Multispecific antibodies may be specific for different epitopes of a polypeptide or may be specific for both a polypeptide as well as for a heterologous epitope, such as a heterologous polypeptide or solid support material.

The terms “protein” and “polypeptide” are used interchangeably and refer to any polymer of amino acids (dipeptide or greater) linked through peptide bonds or modified peptide bonds. Polypeptides of less than about 10-20 amino acid residues are commonly referred to as “peptides.” The polypeptides of the invention may comprise non-peptidic components, such as carbohydrate groups. Carbohydrates and other non-peptidic substituents may be added to a polypeptide by the cell in which the polypeptide is produced and will vary with the type of cell. Polypeptides are defined herein, in terms of their amino acid backbone structures; substituents such as carbohydrate groups are generally not specified, but may be present, nonetheless.

The term “sample” refers to any sample comprising or being tested for the presence of a target of a drug of interest. Such a sample includes samples derived from or containing cells, organisms (bacteria, viruses), lysed cells or organisms, cellular extracts, nuclear extracts, components of cells or organisms, extracellular fluid, media in which cells or organisms are cultured in vitro, blood, plasma, serum, gastrointestinal secretions, urine, ascites, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, pleural fluid, nipple aspirates, breast milk, external sections of the skin, respiratory, intestinal, and genitourinary tracts, and prostatic fluid. A sample can be a viral or bacterial sample, a sample obtained from an environmental source, such as a body of polluted water, an air sample, or a soil sample, as well as a food industry sample. A sample can be a biological sample which refers to the fact that it is derived or obtained from a living organism. The organism can be in vivo (e.g. a whole organism) or can be in vitro (e.g., cells or organs grown in culture). A “biological sample” also refers to a cell or population of cells or a quantity of tissue or fluid from a subject. Most often, a sample has been removed from a subject, but the term “biological sample” can also refer to cells or tissue analyzed in vivo, i.e., without removal from the subject. Often, a “biological sample” will contain cells from a subject, but the term can also refer to non-cellular biological material, such as non-cellular fractions of blood, saliva, or urine. The biological sample may be from a resection, bronchoscopic biopsy, or core needle biopsy of a primary, secondary or metastatic tumor, or a cellblock from pleural fluid. In addition, fine needle aspirate biological samples are also useful. In one embodiment, a biological sample is primary ascites cells. Biological samples also include explants and primary and/or transformed cell cultures derived from patient tissues. A biological sample can be provided by removing a sample of cells from subject, but can also be accomplished by using previously isolated cells or cellular extracts (e.g. isolated by another person, at another time, and/or for another purpose). Archival tissues, such as those having treatment or outcome history may also be used. Biological samples include, but are not limited to, tissue biopsies, scrapes (e.g. buccal scrapes), whole blood, plasma, serum, urine, saliva, cell culture, or cerebrospinal fluid. The samples analyzed by the compositions and methods described herein may have been processed for purification or enrichment of extracellular vesicles contained therein. In one embodiment, the sample is blood.

The term “resist” refers to both a thin layer used to transfer an image or pattern to a substrate which it is deposited upon. A resist can be patterned via lithography to form a (sub)micrometer-scale, temporary mask that protects selected areas of the underlying substrate during subsequent processing steps, typically etching. The material used to prepare the thin layer (typically a viscous solution) is also encompassed by the term resist. Resists are generally mixtures of a polymer or its precursor and other small molecules (e.g. photoacid generators) that have been specially formulated for a given lithography technology. Resists used during photolithography, for example, are called “photoresists.” Resists used during electron-beam lithography are called “ebeam resists.”

The term “drug,” refers to any molecule that can be used in a patient for diagnostics or therapeutic purposes. In some embodiments the drug is a non-protein chemical compound. In some embodiments the drug is a protein, for example, a polypeptide, an antibody or a functional fragment thereof.

### Extracellular Vesicles (EVs)

Extracellular vesicles (EVs) are nanoscale membrane vesicles actively secreted by a variety of mammalian cells, and most notably by rapidly dividing cancer cells (Refs. 10 and 11). These vesicles abound in blood, play important roles in mediating intercellular communication (Refs. 12, 13), and contain a trove of reflective molecular contents inherited from the parent cells (e.g., proteins (Refs. 14, 15), nucleic acids (Refs. 16, 17), lipids as well as various modifications (Refs. 18, 19)). These vesicles are shed by eukaryotic cells, or budded off of the plasma membrane, to the exterior of the cell. These membrane vesicles are heterogeneous in size with diameters ranging from about 10 nm to about 5000 nm. The small vesicles (approximately 10 to 1000 nm, preferably 30 to 100 nm in diameter) that are released by exocytosis of intracellular multivesicular bodies or outward budding of plasma membrane are referred to in the art as “extracellular vesicles”. See, Cocucci et a., (2015). The methods and compositions described herein are equally applicable for other vesicles of all sizes.

The application provides useful methods and compositions related to technology, referred to herein as ExoSCOPE, which analyzes the drug-bound proteins on EVs to molecularly characterize specific drug-target interactions, even of solid tumors. In some embodiments, a plasma sample from a patient is used directly for the ExoSCOPE analysis, without the need for isolating EV's from the rest of the plasma sample, as described below. In some embodiments, EVs can be isolated from in vitro cell culture, e.g., tumor cells lines, as described below. In some embodiments, EVs can be isolated from a bodily fluid (e.g., a blood sample) or a sample prepared from a tissue (e.g., a tumor biopsy) from a patient by differential centrifugation. This method typically employs a series of centrifugation steps with increasing centrifugal force to separate the extracellular vesicles from cells, cell debris and other larger cellular particles. In one embodiment, the blood sample can be first centrifuged at 10,000 g to remove any debris and/or apoptotic bodies and subsequently at 100,000 g to precipitate EVs. The extracellular vesicles are then collected, washed and resuspended in suitable buffer, e.g., PBS. If needed, the extracellular vesicles so prepared can be stored at −80° C. for future usage. In some embodiments, extracellular vesicles can be isolated using a size exclusion chromatography. Suitable size exclusion chromatography is commercially available, for example, sepharose 2B columns, available from Sigma Aldrich (St. Louis, MO). The columns are prepared according to manufacturer's instructions.

EVs prepared as above can be confirmed based on the presence of EV molecular and biochemical markers, using methods well known in the art. In one embodiment, the presence of extracellular vesicles can be confirmed using flow cytometry to analyze markers associated with EVs, e.g., CD63 and CD81. The concentration and size distribution of the EVs can be analyzed using the devices commercially available, for example, the nanoparticle tracking analysis (NTA) system (Nanosight, NS300). In one embodiment, the presence of extracellular vesicles can be confirmed using Western blots to detect EV proteins described above. In some embodiments, size and morphology of the extracellular vesicles can be confirmed using methods such as flow cytometry and transmission electron microscopy.

In methods and compositions disclosed herein, the EVs obtained from patient samples are captured on a sensor. The interaction of drug and its target on the EVs are analyzed using bio-orthogonal probes that are competitive with the drug in binding to its target, as further discussed below.

### Bio-Orthogonal Probe

Aspects of the invention involve bio-orthogonal probes that can be used to label target proteins. As used herein, a bio-orthogonal probe refers to a molecule that binds to a target protein (e.g., the EGFR protein) and comprises a chemically tractable tag enable label to enable label visualization or in situ enzymatic amplification. In some embodiments, the probes were developed for competitive, in situ target labeling in whole extracellular vesicles; this probe labeling can be enzymatically amplified to reflect EV drug occupancy. In some embodiments, the probes are used for rapid, sensitive and specific detection of EGFR proteins and the EGFR drug-target interactions in various settings (cell lysate, live cells, extracellular vesicles and plasma samples).

A bio-orthogonal probe disclosed herein typically comprise a core structure for competitive binding with the drug to the target on the EVs, a chemical the tractable tag, and a linker connecting the chemical the tractable tag and the core structure.

### Core Structure for Competitive Binding

The bio-orthogonal probes are designed to be able to compete with a drug of interest in binding to its target. In some embodiments, the bio-orthogonal probe comprises a core structure that closely resembles the drug such that it is capable of competing with the drug in binding its cognate site in the target. For example, the bio-orthogonal probe may bind to the same site or near the same site on the target as the drug. For purposes of this disclosure, the site on the target that the drug binds are referred to as the cognate site of the drug. In some embodiments, the bio-orthogonal probe confers specific covalent binding to cognate site of the drug on the target and thus blocks the drug from accessing the cognate site. In some embodiments, the bio-orthogonal probe does not confer specific covalent binding to cognate site of the drug on the target, but blocks the drug from accessing its cognate site through other means, e.g., steric hindrance.

Methods for designing probes having the core structure of the drug of a known structure so that it can compete with the drug are well known. In some cases the molecular interaction between the drug and the target are analyzed and probes are then designed based on the three dimensional configurations. For example, the cognate site of the drug on the target can be identified by from the crystal structure of the drug in complex with the target. Various bio-orthogonal probes that can bind to the cognate site on the target can also be designed by using computer modeling, for example covalent docking using flexible side chain method at the cognate site of the target. Software packages for performing such modeling are well known and available, for example, AutoDock Vina, visualized by PyMOL (version 2.3.2).

In some embodiments, the target is EGFR and the drug is an EGFR inhibitor. Non-limiting examples of EGFR inhibitors include small-molecule tyrosine kinase inhibitors, such as gefitinib, erlotinib, afatinib, osimertinib, and icotinib, dacomitinib, CNX2006, and WZ4002. Gefitinib, erlotinib, and icotinib bind reversibly to EGFR and thereby inhibit both the mutant and the wild type EGFR. In contrast, Afatinib and Osimertinib bind covalently and irreversibly blocks EGFR signaling. Xu et al., Comparative review of drug-drug interactions with epidermal growth factor receptor tyrosine kinase inhibitors for the treatment of non-small-cell lung cancer, Onco Targets. Ther. 2019; 12: 5467-5484. Other EGFR inhibitors that can be used in the present methods and compositions include dacomitinib, WZ4002, CNX2006, as disclosed in FIG. 23.

In one embodiment, the bio-orthogonal probe is capable of competing with afatinib in binding to the cognate site in EGFR. In some embodiments, the cognate site is the adenosine triphosphate (ATP) binding pocket of the tyrosine kinase domain of EGFR, a well-known druggable target. See, Kumar et al., (2008), the relevant disclosure is herein incorporated by reference. It is a catalytic domain of protein kinase that relies on ATP as substrate, and the binding of drug will compete with ATP and hence inhibit kinase activity The cognate site of afatinib on EGFR comprise Cys797. Various probes can be designed to compete with afatinib to bind to EGFR at cognate site. In some embodiments, the bio-orthogonal probe is synthesized based on the core structure of afatinib (BIBW2992) (Li et al. (2008)) to confer specific covalent binding to the EGFR kinase site. Exemplary procedures used to produce a bio orthogonal probe is described in FIG. 11 and also Example 8. In some embodiments, the bio-orthogonal probe that competes with afatinib has a structure of formula I below.

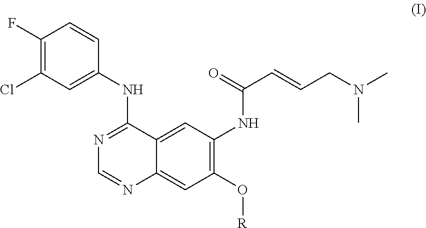

In some embodiments, R contains a chemically tractable tag and a three-carbon linker. In some embodiments, R is selected from the group consisting of

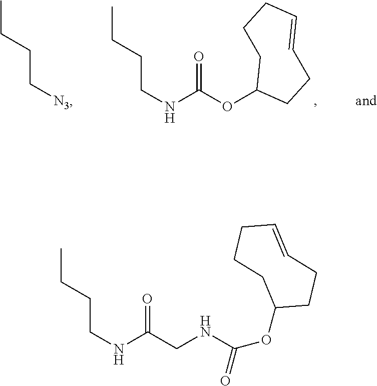

In some embodiments, the probe comprises or consists of the structure of A1:

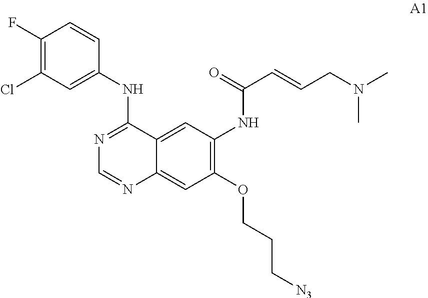 [SMILES: CN(C)C/C=C/C(=O)Nc1cc2c(Nc3ccc(F)c(Cl)c3)ncnc2cc1OCCCN=[N+]=[N-]]

In some embodiments, the probe comprises or consists of A2: NH

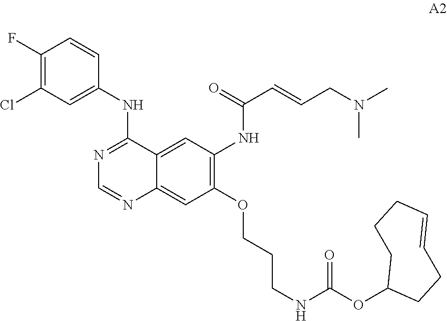 [SMILES: CN(C)C/C=C/C(=O)Nc1cc2c(Nc3ccc(F)c(Cl)c3)ncnc2cc1OCCCNC(=O)OC1CC/C=C/CCC1]

In some embodiments, the probe comprises or consists of A3:

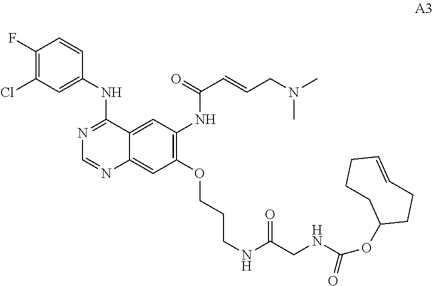 [SMILES: CN(C)C/C=C/C(=O)Nc1cc2c(Nc3ccc(F)c(Cl)c3)ncnc2cc1OCCCNC(=O)CNC(=O)OC1CC/C=C/CCC1]

### Chemically Tractable Tag and Signaling Amplification

The bio-orthogonal probe further comprises a chemically tractable tag to enable visualization and/or in situ enzymatic amplification of the signal resulted from binding of the bio-orthogonal probe to the EVs. The chemical tractable tag used herein can be any molecule that is detectable. In some embodiments, the chemical tractable tag is one that can participate in a chemical reaction to produce an insoluble optical product that can be detected, and the detected signal corresponds to the binding of the click probe to the target.

In some embodiments, the chemically tractable tag is ligated to a reporter (e.g., Cy5) through a click chemistry. A bio-orthogonal probe comprising the chemically tractable tag that can be ligated to a reporter through click chemistry is referred to as a click probe in this disclosure. In some embodiments, the click chemistry is a rapid copper-free bio-orthogonal ligation (also known as copper-free click chemistry or copper free click reaction) (Jewett, J. C. & Bertozzi, C. R. (2010); Patterson et al. (2014)). In some embodiments, the chemical tractable tag is able to participate in a copper-free click chemical reaction. Non-limiting examples of the chemical tractable tags include an azide, and a trans-cyclooctene (TCO). (Refs. 24 and 25). In some embodiments, the R group of a probe according to formula I comprises an azide (for example, probe A1), which can be conjugated to dibernzocyclooctyne (DBCO). Thus, the click probes having this structure can be used to recruit a DBCO-conjugated reporter, e.g., a DBCO-conjugated Cy5. In some embodiments, the R group comprises a trans-cyclooctene (TCO), which can be directly conjugated to a tetrazine. The click probes having this structure can be used to recruit a tetrazine-conjugated reporter. Various probes and the respective reporters that they ligate to are shown in FIG. 12.

Besides Cy5, any other reporter that is capable of producing a detectable signal can be used to ligate to the chemically tractable tag, for example, those produce fluorescent, such as, include rhodamine, tetramethylrhodamine (TMR, TMARA) or luminescent signals. Examples of reporters may also include affinity reporters (e.g., biotin) or enzymatic reporters (e.g., horse radish peroxidase). Examples of reporters are also described in Rutkowska et al. (2016), the relevant disclosure is herein incorporated by reference.

In some embodiments, the bio-orthogonal probe further comprises a linker (e.g., a carbon linker) connecting the chemically tractable tag and the rest of the probe (e.g., the core structure). For example, the R group in formula (I) comprises or consists of a carbon linker and one or more chemical tractable tags. In preferred embodiments, the carbon linker used in the probe is short in length so that it does not interfere with the biological properties of the probe. In some embodiments, the carbon linker has no more than 10 carbons. In some embodiments, the carbon linker has 2-6 carbons, e.g., 2-5 carbons, or about 3 carbons.

### Signal Amplification

In some embodiments, the chemically tractable tag can recruit a signal amplifying moiety comprising an enzyme, and enzyme can induce the formation of an insoluble aggregate on the surface on the sensor chip, e.g., by catalyzing in situ conversion of a soluble substrate to form a local insoluble deposits on the probe-bound vesicles (FIG. 7a). These high-density optical deposits, formed in low-drug-occupancy EVs, lead to plasmonic signal enhancement and a red shift in the resultant transmission optical spectrum. This phenomenon is referred to as in situ amplification. In situ amplification increases the sensitivity of the sensor chip by resulting in a greater change in transmission wavelength (spectral shift) or change in transmission intensity when a second recognition molecule binds to an analyte on the surface of the sensor chip.

The enzyme may be horse radish peroxidase (HRP), alkaline phosphatase, glucose oxidase, 3-lactamase or β-galactosidase or an enzymatic fragment thereof. In one embodiment, the enzyme is horse radish peroxidase. In one embodiment, the first biorecognition molecule is fused to the signal amplification moiety. For example, the first biorecognition molecule may be an antibody that is covalently fused to a horse radish peroxidase enzyme that is covalently linked to the antibody using techniques that are well known in the art.

The method may further comprise contacting the enzyme with an enzyme substrate. The enzyme substrate may be one that could form an insoluble product in the presence of enzymes or upon enzymatic action. For horse radish peroxidase (HRP), formulations such as 3-amino-9-ethylcarbazole, 3,3′,5,5′-Tetramethylbenzidine or Chloronaphthol, 4-chloro-1-naphthol can be used. These substrates are able to turn into an insoluble product upon enzymatic reaction of HRP. In one embodiment, the enzyme substrate is 3,3′-diaminobenzidine tetrahydrochloride.

In some embodiments, similar to ligating to a reporter described above, the chemically tractable tag recruits the signal amplifying moiety through a click chemistry, e.g., a copper-free click chemical reaction. In some embodiments, a click probe comprise azide as the chemically tractable tag, which can be readily ligated to an enzyme to that is conjugated to dibernzocyclooctyne (DBCO). Thus, the click probes having this structure can be used to recruit a DBCO-conjugated enzyme, e.g., a DBCO-conjugated HRP. In some embodiments, a click probe comprises a trans-cyclooctene (TCO) as the chemically tractable tag, which can be directly conjugated to a tetrazine via click chemistry. Click probes having this structure can be used to recruit a tetrazine-conjugated enzyme, e.g., a tetrazine-conjugated HRP.

### Evaluating the Properties of the Bio-Orthogonal Probes

The bio-orthogonal probe competes with the drug in binding to the target on the EVs. The ability of the probe to compete with the drug can be determined using a competitive binding assay. In some embodiments, a competitive binding assay is set up in which the bio-orthogonal probe incubated with cells or EVs expressing the target, e.g., EGFR, in the presence of varying concentrations of the drug (e.g., afatinib). A drug dose-dependent decrease in the probe labeling of the target indicates the probe competes with the drug (i.e., the probe is competitive to the drug in binding the target). In some embodiments the IC₅₀ of the drug from the competitive binding assay (i.e., the concentration of the drug used which corresponds to 50% of signal from probe's labeling of the target in the absence of the drug) is in the range of 0.5 nM to 10 nM, e.g., the 1.0 nM to 5 nM, or about 1.4 to 1.6 nM. Assays for conducting such competitive binding include, but are not limited to, in-gel fluorescence, flow cytometry, and click ELISA. One illustrative experiment in FIGS. 2c and 2e showed that probe A3 competed with afatinib and the IC₅₀ of the afatinib was 1.4 to 1.6 nM.

Optionally, a bio-orthogonal probe disclosed herein possess substantially similar functional activity as the drug that it competes with. The term “substantially similar functional activity,” as used herein, refers to the functional activity of the probe is 50% to 500% of that of drug when measured under the same assay conditions. For example, if the drug has a role of inhibiting proliferation (“anti-proliferation”) of cancer cells, the bio-orthogonal probe and the drug can be evaluated in a proliferation assay of the target cell lines, and the inhibition function on proliferation can be evaluated. In some embodiments, the anti-proliferation function is measured by a GI₅₀. As used herein, GI₅₀ measures the anti-proliferation function of the drug or the bio-orthogonal probe. GI₅₀ equals to the concentration of the drug or probe used to cause 50% inhibition of proliferation. A lower GI₅₀ indicates a higher potency, i.e., a higher anti-proliferation activity. In some embodiments the GI₅₀ of the bio-orthogonal probes is in a range of 50%-500% of the GI₅₀ of the drug itself, e.g., 60%-300%, 100%-400%, or 100%-300%. As compared to A2, A3 showed enhanced anti-proliferation activity on human lung cancer cells H3255. See FIG. 2B. And as compared to A1 and A2, A3 demonstrated the highest signal-to-noise ratio in terms of target (e.g., EGFR) labeling. See FIG. 2C and Example 3. One illustrative example is shown in FIG. 2B, in which the probe A3 shows a GI₅₀ of 1.8 nM as compared to Afatinib, which has a GI₅₀ of 0.6 nM.

Optionally, the bio-orthogonal probe is also selected based on its lipophilicity. Lipophilicity affects the solubility, permeability, potency, selectivity, absorption, distribution of the probe. Typically, a higher lipophilicity is associated with higher permeability but lower solubility. It is desirable to have probes having an optimal lipophilicity such that it can enter the EVs to bind the target with high potency. In some cases, it is desirable to have probes that have properties similar to that of the drug (e.g., afatinib) to achieve competition in binding to the target (e.g., EGFR). Lipophilicity of the probes can be evaluated by a distribution coefficient, cLogD (also referred to as Log D). A higher value of cLogD indicates a higher lipophilicity. Methods to measure the distribution coefficient are well known, for example, as described in Csizmadia F, et al. (1997). In some embodiments, the probe has a cLogD that in range of 60% to 400% of the value of the drug it competes with in binding the target on the EVs under the same assay conditions, for example, 70% to 300%, 80% to 200%, 90% to 180%. In some embodiments, the same conditions include the same pH, e.g., pH 7.4. As compared to A2, A3 showed improved lipophilicity that is closer to that of the drug, Afatinib. See FIG. 2B. And as compared to A1 and A2, A3 demonstrated the highest signal-to-noise ratio. See FIG. 2C and Example 3 below.

A1-A3 compete with afatinib in binding to EGFR. Exemplary methods of using these probes to monitor drug occupancy of patients who have been administered with the drug are shown in, e.g., Example 3, FIG. 2-3 and FIG. 14.

### Manufacturing the Click Probes

In general, synthesis of the click probes can begin with an intermediate product that is used to produce the drug of interest, e.g., Afatinib. Carbon linkers containing a Boc-protected amine is incorporated which is followed by removing the Boc protection group and the resulting amino group can be converted to azide or different trans-cyclooctene TCO building blocks to produce click probes. NMR analysis can be performed during each step of synthesis to confirm the formation of the product.

Example 8 and FIG. 11 show an illustrative example of synthesis of click probes that are competitive to afatinib. The synthesis began with a commercially available intermediate 1, which is commonly used for afatinib preparation. Firstly, substitution reaction was performed on 1 to incorporate a 3-carbon linker containing a Boc-protected amine. Next, the nitro group on 2 was reduced to amine and then functionalized with the Michael acceptor as afatinib. Finally, the Boc protection group on 3 was removed and the resulting amino group was either converted to azide (A1) or coupled with different trans-cyclooctene TCO building blocks to yield the click probes (A2, A3) accordingly. The purity of click probes was confirmed with high performance liquid chromatography (HPLC) and high-resolution mass spectrometry (HRMS). See Example 8 for details of synthesis and NMR characterization of probes A1, A2, and A3.

### Sensor

The methods and compositions of the disclosure use a sensor to detect interactions among the bio-orthogonal probe, EVs, and the capture agent on the sensor. The sensor is a plasmonic sensor, which generates a plasmonic resonance and/or plasmonic coupling when illuminated by an optical source. Plasmonic resonance may be influenced by factors such as materials and geometric features, causing an enhanced electromagnetic field distribution near the dielectric interface. Plasmonic coupling occurs when two or multiple resonating structures are brought closely together, such that their resonant field start to interact and hybridize. Factors influencing plasmonic coupling include materials, geometric features of individual structures, and inter-structure distancing. See Jain et al. (2010); Hugall et al. (2018). Typically, the sensor comprises at least a conductive layer that is deposited above a substrate layer. The conductive layer can be any metal layer, for example, gold, copper, titanium, aluminum, and chromium. The substrate {Aizpurua, J. et al. Optical properties of gold nanorings. Phys Rev Lett 90, 057401 (2003).} can be, for example, a polymeric substrate, a glass substrate (Sio2), or the like.

The conductive layer comprises nanostructures that are patterned to form nanogaps (nanovoids) among them. The nanogaps are of appropriate sizes such that they provide spaces to capture optical energy and produce plasmonic resonance and/or plasmonic coupling. These nanostructures are also referred to as nanoresonators or plasmonic nanoresonators. In some embodiments, the average size of the nanogaps are in the range of 20 to 500 nm, for example, 20-200 nm, 50 to 450 nm, 100 to 400 nm, or 150 nm to 250 nm, or about 200 nm. In some embodiments, the thickness of the nanostructures are in the range of 20 nm-200 nm, 20 nm-100 nm, 30 nm-90 nm, or about 50 nm. FIG. 10 illustrates the thickness, outer diameter, and the inner diameter of an exemplary nanostructure of the sensor.

In some embodiments, the nanogaps in the sensor are of uniform size. In some embodiments, they are of different sizes, i.e., at least some of the nanogaps are of different size from other nanogaps. In some embodiments, the nanogap structures are patterned on the substrate to form a periodic lattice. As used herein, periodic lattice refers to a network of nanostructures (e.g., nanorings) arranged in a uniform and periodic pattern.

As used herein, the term “periodicity” refers to the recurrence or repetition of nanostructures at regular intervals by their positioning on the sensor chip. The term “periodic” thus refers to the regular predefined pattern of nanostructures with respect to each other. The regular periodicity among the nanorings (i.e., the distance between the adjacent nanorings) may allow the tight control of the resonance wavelength and penetration of the evanescent wave. In one embodiment, the nanostructures have a periodicity of about 250 nm to about 650 nm. In one embodiment, the nanostructures have a periodicity selected from the group consisting of 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm and 650 nm or anywhere in between. In one embodiment, the nanostructures have a periodicity of 450 nm.

In some embodiments, the nanostructures comprised in the conductive layer are nanorings. In some embodiments, the conductive layer is a gold layer and the nanorings so formed are referred to as gold nanorings. Each nanoring comprises a nanoring gap, which is defined by an outer circular shape and an inner circular shape and the size of the nanoring gap equals to the half of the difference between the outer circle diameter and the inner circle diameter. The outer circular shape has an outer diameter in a range from 200 nm to 500 nm, and/or the inner circular shape has a inner diameter in a range from 30 nm to 250 nm. In some embodiments, the thickness of the nanorings is in the range of 20 nm-200 nm, e.g., 20 nm-100 nm, 30 nm-90 nm, or about 50 nm. FIG. 10 illustrates the thickness, outer diameter, and the inner diameter of an exemplary nanostructure of the sensor. In one illustrative example is shown in FIG. 10, a sensor comprising nanoring resonators patterned in a gold film with dimensions of 50 nm (thickness), 150 nm (in ring diameter) and 350 nm (outer ring diameter) showed uniform fabrication and consistent optical performance across sensors.

In some embodiments, the sensor used in this application comprises an array of sensing elements, and this type of sensor is also referred to as a sensor array in this disclosure. In some embodiments, each sensing element comprises a spatially-optimized nanorings.

As shown in FIG. 16, a periodic lattice of nanorings showed higher signal amplification as compared to nanoholes, suggesting that sensors using nanoring resonators are more sensitive in signal detection. Field simulation experiments showed that the electromagnetic fields within the nanoring gap (“in-ring”) of the nanoring structures, were stronger than as compared to that on the sensor surface (“atop”) (FIG. 3B, left). These results show that the sensor comprising periodic lattices of gold nanorings have improved spatial control for signal amplification. Specifically, field simulations showed that enhanced electromagnetic fields are located within the nanoring gap (in-ring).

To leverage this in-ring field confinement, extracellular vesicle molecular reactions can be patterned and performed within the nanoring gaps. The design of the device (e.g., inner diameter, outer diameter) can be optimized for enhanced SPR transmission intensity and detection sensitivity as described herein. In some embodiments, a click probe is designed such that it can be used for competitive, in situ target labeling in whole extracellular vesicles; this probe labeling can be enzymatically amplified to reflect EV drug occupancy, as further disclosed herein. In some embodiments the sensor comprise an array of sensing elements different sensoring elements are in ring functionalized with different capture agents, such that the sensor can be used for multiplex detection, as further discussed below.

### Sensor Fabrication

In some embodiments, a microarray chip containing a large number of sensing elements can be fabricated for high-throughput, multiplexed analysis, as well as parallel measurements of multiple biomarkers. In some embodiments, the chip is a microarray nanoring sensor chips with an improved and coupled optical performance which is robust Methods for manufacturing arrays having sensing elements are described in, for example, Xin et al. (2018), entire content of which is herein incorporated by reference. In some embodiments, the sensor may be fabricated using one or more of the following steps. A glass substrate can be coated with PMMA 495 k, and additional layers of Espacer to improve substrate conductivity. lithography (EBL, Joel 6300FS) may be performed to define the nanoring pattern in the resist before development in a suitable organic solvent. An adhesion layer may also deposited onto the substrate that bear the nanoring pattern. Optionally, a lift-off process in solvent stripper is performed. The dimensions of the nanorings can be characterized by microscopy, e.g., scanning electron microscopy and/or atomic force microscopy. The nanorings of the sensor then can be functionalized with specific molecules for detection of certain molecules on the EVs.

### Channel Assembly

Standard lithography can be used to fabricate a multichannel flow cell that comprises channels for delivering reagents to the sensor. One exemplary procedure of channel assembly is shown in Example 1. A SU-8 negative resist is spin-coated on a Si wafer and then baked at high temperature briefly, for example at 65 C and 95 C for 1 and 6 min. In some cases, after UV light exposure, the resist is baked again before being developed under agitation. The developed wafer can then be rinsed and dried. In some instances, the resist is then chemically treated by e.g., trichlorosilane vapor inside a desiccator for 15 min, and treated by polydimethylsiloxane polymer (PDMS) and cross-linker that are mixed at a suitable ratio (e.g., a ratio of 10:1). The treated SU-8 mold is then cured at a high temperature (e.g., in an oven at 75° C. for 30 min). The PDMS layer can be cut from the SU-8 mold and assembled onto the ExoSCOPE sensor. The channels are processed to have inlets and outlets with dimensions suitable for sample processing.

### Optical Setup and Analysis

In some embodiments, a light source is provided to illuminate the ExoSCOPE sensor. Transmitted light is then collected and fed to a detector (e.g., a spectrometer) and the intensity of the light can be recorded in counts against wavelength.

In some embodiments, the spectral peaks of the transmitted light can be analyzed using a software package suitable for this purpose, for example, a custom-built R program by fitting the transmission peak using local regression method.

### Microfluidic System

Any one of the assay workflows disclosed herein can be implemented in a microfluidic system. In some embodiments, the microfluidic system comprises a flow cell housing the sensor array comprising a plurality of sensing elements. The system may further comprise microfluidic channels for introducing samples into the sensor array. Optionally the system provides a light source to illuminate the sensor array. In some embodiments, the microarray chips are pre-functionalized with capture agents (e.g., antibodies against cancer markers) to enable rapid and sensitive readouts, without requiring extensive sample processing and is thus suitable for targeted clinical measurements.

The microfluidic implementation disclosed herein facilitates parallel workflow and enables small volume of samples to be used for detection with the developed platform.

### Detecting Binding of EVs and Probes to the Sensor

The detection of “binding” of the EVs or the click probe to the captured agent on the surface of a sensor may be via a spectral shift in terms (change in transmission wavelength) or a change in transmission intensity at a fixed wavelength. For example, an EV that is captured on a surface of a sensor chip will have an initial reference wavelength. Upon binding and amplification of a bio-orthogonal probe, as described above, the associated transmission spectrum may shift to a longer wavelength.

The change in transmission resonance wavelength (or spectral shift (AX)) or change in transmission intensity at a fixed wavelength in a sample may be compared to the change that is observed in a control sample. This may be used to, for example, determine whether there is increased binding of a bio-orthogonal probe to the captured EV.

The “increased binding of the bio-orthogonal probe” in a sample as compared to a control sample may be determined by comparing the change in spectral shift, or a change in transmission intensity at a fixed wavelength, between the sample and the control sample upon binding of the second recognition molecule. An increased change in spectral shift or change in transmission intensity may indicate that there is an increased binding of the second recognition molecule to the analyte.

In one embodiment, the increased change in spectral shift or transmission intensity may refer to a 1.2-fold or greater increase between the subject and the control subject. The term may also refer to an increase that is selected from a group consisting of 1.1 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, 29 fold, 30 fold, 31 fold, 32 fold, 33 fold, 34 fold, 35 fold, 36 fold, 37 fold, 38 fold, 39 fold, 40 fold, 41 fold, 42 fold, 43 fold, 44 fold, 45 fold, 46 fold, 47 fold, 48 fold, 49 fold, 50 fold, 51 fold, 52 fold, 53 fold, 54 fold, 55 fold, 56 fold, 57 fold, 58 fold, 59 fold, 60 fold, 61 fold, 62 fold, 63 fold, 64 fold, 65 fold, 66 fold, 67 fold, 68 fold, 69 fold, 70 fold, 71 fold, 72 fold, 73 fold, 74 fold, 75 fold, 76 fold, 77 fold, 78 fold, 79 fold, 80 fold, 81 fold, 82 fold, 83 fold, 84 fold, 85 fold, 86 fold, 87 fold, 88 fold, 89 fold, 90 fold, 91 fold, 92, fold, 93 fold, 94 fold, 95 fold, 96 fold, 97 fold, 98 fold, 99 fold and 100 fold.

### Exoscope

ExoSCOPE represents an assay format and system supporting the assay used in extracellular vesicle monitoring of drug occupancy and protein expression. ExoSCOPE utilizes bio-orthogonal probe amplification and spatial patterning of molecular reactions within matched plasmonic nanoring resonators to achieve in situ analysis of EV drug dynamics.

In some embodiments, ExoSCOPE is used to evaluate drug occupancy of a subject who has been treated with a drug. EVs from a biological sample (e.g., a plasma sample) from patients who have been treated with the drug (e.g., an EGFR inhibitor) are collected and immobilized onto the sensor that have been functionalized with a capture agent that can bind to the EVs. In some embodiments, the capture agent is an antibody. In some embodiments, the capture agent is anti-CD63, CD81, or CD9. In some embodiments, the capture agent is an antibody against a cancer marker, e.g., HER2, MUC1, EpCAM and EGFR.

### In Ring Functionalization and Binding of EVs to the Sensor

The EVs are captured on the sensor via a capture agent immobilized on the sensor. A capture agent can be any molecule that binds to a target on the EVs. Immobilizing the capture agent on the sensor is also referred to as functionalization of the sensor. In some embodiments, at least 70%, at least 80%, at least 85%, at least 90%, or at least 95% of the capture agent molecules are immobilized on the substrate in the nanoring, referred to as “in—ring functionalization”. As shown in FIG. 3B left, field simulations showed that the enhanced electromagnetic fields were located within the nanoring gap (“in-ring”) as compared to that on the sensor's conductive surface (“atop”) (FIG. 3B, left). As shown in FIG. 17, as compared to atop functionalization, in ring functionalization showed superior performance. As discussed below, in some embodiments, the nanoring gaps of each sensing element are in ring functionalized with molecules of a capture agent. In some embodiments, the sensor comprises multiple sensing elements and at least one sensing element is in ring functionalized with a capture agent that is different from another sensing element.

In one embodiment, the capture agent is an antibody. The antibody may, for example, be an antibody that recognizes a pan-EV biomarker or a marker that is associated or bound to an EV. For example, the antibody may be an antibody that is specific to CD63, LAMP-1, Alix, HSP90, Flotillin 1, TSG101, CD9 or CD81, which are abundant and characteristic in EVs. The antibody may also be specific to a cancer marker such as HER2, EGFR, EpCAM, and MUC1.

The capture agent may be immobilized on the sensor using techniques that are well known in the art. For example, the capture agent may be adsorbed onto the surface. Alternatively, the surface may be coated with a layer of streptavidin or avidin prior to immobilization of the capture agent. The capture agent molecule may be biotinylated and immobilized onto the surface via streptavidin-biotin conjugation. In one embodiment, the surface may be treated with polyethylene glycol (PEG) molecules. The surface may be treated with an active (carboxylated) thiol-PEG. The surface may then be activated through carbodiimide crosslinking in a mixture of excess NHS/EDC dissolved in MES buffer and conjugated with the capture agent molecule. In an alternative embodiment, the surface may be treated with a mixture of polyethylene glycol (PEG) containing long active (carboxylated) thiol-PEG and short inactive methylated thiol-PEG. The ratio of long active (carboxylated) thiol-PEG to short inactive methylated thiol-PEG can be optimized for maximal functional binding. The surface may then be activated through carbodiimide crosslinking in a mixture of excess NHS/EDC dissolved in MES buffer and conjugated with a capture agent molecule.

The capture (binding) of the EVs to the sensor surface may result in a spectral shift in terms of change in transmission wavelength or a change in transmission intensity at a fixed wavelength. This signal can be detected by the sensor as discussed above and recorded as signal M.

### Binding of the Bio-Orthogonal Probe to the EVs Captured on the Sensor

The bio-orthogonal probe described above can be introduced to the flow cell and allowed to contact the sensor. The bio-orthogonal probe is competitive to the drug, and it will bind to the drug's target molecules on the EVs unless they are occupied by the drug. Such binding will cause a spectral shift in term of change in transmission wavelength or a change in transmission intensity, and the binding can be detected and recorded as signal P.

In some embodiments, a signal amplifying moiety is added, which is ligated to the bio-orthogonal probe via click chemistry. Appropriate substrates are added to the sensor and can react with the signal amplifying moiety to produce insoluble optical product deposited on the sensor, which leads to plasmonic signal enhancement and a red shift in the resultant transmission optical spectrum. In these embodiments, signal P refers to the signal corresponding to the signal resulted from the signal amplification.

In some embodiments, a labeling index is defined based on the marker-specific EV binding (signal M) and the probe-induced amplification signal (signal P) in the same vesicles to account for differences in vesicle counts and composition across samples. In some embodiments, μ=kP/M, where k is a coefficient constant. In some embodiments, k=1. This labeling index corresponds to the average probe density per sensor-captured vesicle. (FIG. 7). Thus, in some embodiments a labeling index can be used to indicate the expression level of the target in the EVs. In one illustrative examples, the ExoSCOPE method was used to measure the labeling index of EVs derived from various cell lines with known EGFR expression (FIG. 18e). The results confirmed that the analyses indeed reflect vesicular EGFR expression levels (FIG. 3d and FIG. 18f).

The ExoSCOPE assay is sensitive and highly specific; it can be directly performed on plasma samples without the need for isolating EVS therefrom. In one illustrative embodiment ExoSCOPE measurements performed directly in plasma samples showed a high specificity, as indicated in that the ExoSCOPE measurement remained similar when the sample were spiked in PBS or plasma (FIG. 18c-d). In some embodiments, the assay has a limit of detection (LOD) of about 1000 probe-labeled EVs, which is 10e4 fold better than of the click ELISA assay. See FIG. 3c. In some embodiments, the ExoSCOPE assay can be performed on scant exosome sample (e.g., 5 μL of native plasma) to measure multiparametric drug dynamics (i.e., protein composition and drug occupancy changes). and can be completed within one hour. In some embodiments, ExoSCOPE can detect even delicate changes of drug interaction with different mutant proteins.

The ExoSCOPE assay can be used in a number of applications, such as determining drug occupancy, screening for drug that is suitable to the patient, determining whether a patient has a mutation in a target that would affect the drug treatment, and diagnosing cancer in a patient, as further described below.

### Method of Determining Drug Occupancy (Treated Evs)

The methods and compositions disclosed herein can be used to determine drug occupancy over time. Drug occupancy as used herein, refers to that percentage of the target molecules (e.g., EGFR) on the cell that are bound by a drug (e.g., afatinib). For a drug the mechanism of action is through binding to a target on the tumor cell, a higher drug occupancy indicates that the drug is more likely to be effective. In some embodiments, the method measures binding of a drug to a target in a subject that has been treated with a drug over a treatment period. In some embodiments, the drug occupancy can be determined within 24 hours after the drug administration, which provides fast and accurate determination whether the drug is effective and aid in the selection of the most suitable treatment plan for the patient.

As shown in Example 5, the inventors have discovered a strong correlation between the drug occupancy of the cell and drug occupancy of the EV, indicating that the ExoSCOPE analysis can reflect cellular drug occupancy in real time. Thus, in some embodiments, the method measures the binding of a drug to a target in a subject that has been treated with a drug over a treatment period. The method includes providing a probe that is capable of competing with the drug in binding to the target and contacting the probe with extracellular vesicles (EVs) from samples obtained from the subject at different time points of the treatment period and detecting the binding of the probe to the EVs in the samples. A decrease in the binding of the probe to the EVs as treatment period progresses indicates an increase in the binding of the drug to the target in the subject, which indicates the drug is effective. Stated differently, the drug can be determined as effective if the binding of the probe to the EVs at a later time point in the treatment period is higher relative to the binding of the probe to the EVs at an earlier time point. Conversely, the drug can be determined as ineffective if the binding of the probe to the EVs at a later time point in the treatment period is lower relative to the binding of the probe to the EVs at an earlier time point. In some embodiments, the EV samples are taken from the patient at regular intervals from the start of the treatment period, and the detection of the increase in drug occupancy at a later time point as compared to an earlier time point indicates that the drug is effective. In some embodiments, the patient is administered with the drug on a regular interval (e.g., daily, every other day, or every three days) and the EV samples are also taken at regular intervals from the start of the treatment period, e.g., within 24 hours from each of the at least two administrations. In some embodiments, the patient is administered with the drug every 24 hours and the EV samples are obtained between 0.5 and 24 hours, between 5 and 24 hours, or between 8 and 24 hours after every drug administration, but before the next drug administration. In one embodiment, patient blood are drawn at the desired time points described above, and plasma are prepared from these blood samples. The plasma samples contain the EVs and can be collected and stored at −80° C. before use in the ExoSCOPE assay disclosed herein.

In some embodiments, the EVs (e.g., the plasma samples) from patients who have received drug treatment are captured on the sensor by a capturing agent as described above and the EVs are incubated with a probe to determine drug occupancy. Contacting EVs with the probe and capturing the EVs on the sensor may be performed in any order. In some embodiments, the EVs are captured on the sensor and the probe is applied to the EVs that have been captured on the sensor. In some embodiments, the EVs are first incubated with the probe before the probe bound-EVs are captured on the sensor.

In some embodiments, the EVs are first captured before contacting the probe, and the capture of the EVs to the sensor surface result in a spectral shift in terms of change in transmission wavelength or a change in transmission intensity at a fixed wavelength. This signal can be detected by the sensor as discussed above and recorded as signal M. The probe's binding to the target molecules on the EVs (unless they are occupied by the drug) will cause a spectral shift in term of change in transmission wavelength or a change in transmission intensity. A signal amplifying moiety comprising an enzyme is then be added, and the signal amplifying moiety is ligated to the probe via click chemistry as described above. Appropriate substrates are added to the sensor and react with the enzyme to produce insoluble optical product deposited on the sensor. The deposit of the optical product on the sensor causes lead to plasmonic signal enhancement and a red shift in the resultant transmission optical spectrum, which is detected and recorded as signal P. Because the amplification occurs on the sensor where the EVs are bound, this amplification is referred to as in situ amplification of the signal corresponding to the binding of the probe to the target molecule on the EVs.

In some embodiments, the EVs are first incubated with the probe and the probe binds to the EVs which are not fully occupied by the drug. The EVs (including those are bound by the probe and those are not bound by the probe) are captured on the sensor. The capture (binding) of the EVs to the sensor surface results in a spectral shift in terms of change in transmission wavelength or a change in transmission intensity at a fixed wavelength. This signal can be detected by the sensor as discussed above and recorded as signal M. A signal amplifying moiety comprising an enzyme can then be added, which is ligated to the probe. Appropriate substrates are added to the sensor and react with the enzyme to produce insoluble optical product deposited on the sensor. The deposit of the optical product on the sensor lead to plasmonic signal enhancement and a red shift in the resultant transmission optical spectrum, which is detected and recorded as signal P

In some embodiments the probe labeling index μ is determined based on the ratio of signal P to signal M, as described above. In some embodiments, the probe labeling index μ is normalized against a reference probe labeling index μ₀ to produce a normalized probe labeling index. The reference probe labeling index μ⁰ refers to the probe labeling index determined on a control sample, e.g., a sample from a subject that has not been treated with the drug.

Drug occupancy is inversely related the probe labeling index. In one embodiment the drug occupancy index can be represented by the following formula: ξ=1−μ/μ⁰.

A drug occupancy index is useful for a study on longitudinal treatment monitoring, i.e., a study to monitor the effect of a treatment over the entire or a portion of the treatment period. In one embodiment, a drug occupancy index is determined for EV samples taken from patients at a different time points after start of the treatment; an increase or no change in the drug occupancy index at a later time points as compared to the drug occupancy index at an earlier time point indicates that the drug is effective; conversely, a decrease in the drug occupancy index as a later timepoint as compared to the drug occupancy at an early time point indicates that the drug is ineffective.

### Multiplexed Detection of Protein-Typed Extracellular Vesicle Subpopulations

In some aspects, the invention involves multiplexed detection of marker-typed extracellular vesicle subpopulations and respective drug occupancy within these extracellular vesicle subpopulations. Bioassays can be developed for the multiplexed ExoSCOPE workflow to marker-type and measure drug occupancy in molecular subpopulations of extracellular vesicles.

In some embodiments, a multiplex drug occupancy index determination can be performed. For example different subpopulations of EVS are captured on the sensor, and each subpopulation are bound by a different capture agent immobilized on a discrete area on the sensor, such that the EVs bind to two or more different capture agents. Each of the two or more different capture agents can bind to EV subpopulations expressing a different marker, e.g., HER2, EGFR, EpCAM, and MUC1. Drug occupancy can be determined on each of the EV subpopulations at a time point, e.g., within 24 hours, e.g., between 5 and 24 hours, between 8 and 24 hours after the administration of the drug.

A drug occupancy index can be determined for each population and a plurality of occupancy indexes for all EV subpopulations can be generated. Optionally, a composite drug occupancy index can be determined based on the combination of a plurality of occupancy indexes. In one embodiment, the compositions drug occupancy index is generated from the plurality of drug occupancy indexes using a multiple linear regression model. In some embodiments, the compositions drug occupancy index is calculated from at least two, at least three, at least four, or at least five individual drug occupancy indexes using the multiple regression model; each individual drug occupancy is determined as described. For illustration and not for limitation, the following exemplifies to calculate a composition and drug occupancy index based on three drug occupancy indexes, X1, X2, and X3, determined on EV subpopulations that are captured by three different capture reagents. For example, three capture reagents that bind to three different markers, each selected from the group consisting of CD63, CD9, CD81, HER2, MUC1, EpCAM, and EGFR. The clinical RECIST analysis can be used as the outcome variable, a leave-one-out cross-validation is applied to avoid overfitting. That is to say, the prediction score of each patient sample is computed based on the best fit relationship using all other patient data as a training set. Leave-one-out cross-validation is well known, for example, as described in Sammut C., Webb G. I. (eds) Encyclopedia of Machine Learning. Springer, Boston, MA. doi.org/10.1007/978-0-387-30164-8_469. RECIST identified treatment response as Responder (Y=1) or Non-responder (Y=0); so every patient is denoted as (Y, X1, X2, and X3). The model fits multiple patient data and determines the best fit relationship as Y=aX1+bX2+cX3+d, where a, b, c and d are constants. The constants and marker expressions are used to compute the composite score (=aX1+bX2+cX3+d) for each patient from the regression model. As discussed above, a longitudinal treatment monitoring study can be conducted to determine the composite drug occupancy index at different time points after treatment; an increase or no change in the composite drug occupancy index at a later time points as compared to the composite drug occupancy index at an earlier time point indicates that the drug is effective; conversely, a decrease in the composite drug occupancy index as a later timepoint as compared to the compositions drug occupancy index at an earlier time point indicates that the drug is ineffective. One illustrative example of using the composite drug occupancy index to determining the efficacy of lung cancer therapy is shown in Example 6.

Further multiplexed ExoSCOPE on time-dependent changes in EV drug occupancy (Δξ) could effectively distinguish responders from non-responders (P<0.0005) undergoing targeted treatment of EGFR inhibitor. The difference could be observed as early as in 24 hours after treatment initiation, while responder and non-responder status was clinically determined at the end of the treatment (day-21) by tumor volumetric imaging. The other changes in EV protein marker composition (ΔM) or total drug concentration in blood plasma (ΔD) showed insignificant differences between the two clinical groups.

The technology could be directly applied to clinical plasma samples and accurately detected lung cancer patients, providing drug occupancy signatures that could distinguish treatment efficacy.

### Drug Screening

The methods and compositions disclosed carrying can also be used for drug screening. In my embodiments a method provided herein can be used to compare the potency of a first drug relative to a second drug on a subject. In some embodiments the first drug is a test drug of unknown potency and the second drug is a reference drug with known potency in treating the cancer.

In some embodiments, the method comprises contacting EV samples of a subject with the first drug and the second drug used at the same concentration separately. A bio-orthogonal probe that is competitive to the first and the second drug in binding to the target is added to the EVs that have been contacted with the first drug or the second drug. The method further comprises detecting the binding of the probe to the EVs. A lower binding of the probe to the EVs that have been contacted with the first drug relative to the second drug indicates that the first drug is more potent than the second drug. Conversely, a higher binding of the probe to the EVs that have been contacted with the first drug relative to the second drug indicates that the first drug is less potent than the second drug. The binding of the probe to the EVs may be determined based on the probe labeling index or the drug occupancy index, the two inversely related parameters.

In some embodiments, the binding of the probe to the EV's is determined based on the probe labeling index and comparing the potency of the first drug to a second drug involves contacting the EVs with varying concentrations of the first drug and varying concentrations of a second drug, respectively. The method further comprises adding a probe to the EVs that have been contacted with the first or the second drug and determining the probe labeling index at each concentration of the first or second drug. Then an IC₅₀ of the probe labeling index of the first drug and an IC₅₀ of the second drug are calculated. The method then determines the first drug is less potent than the second drug if the IC₅₀ of the probe labeling index of the first drug is lower than the second drug and determines the first drug is more potent than the second drug if the IC₅₀ of the probe labeling index of the first drug is higher than the second drug.

In some embodiments, the binding of the probe to the EV's is determined based on drug occupancy index and comparing the potency of the first drug to a second drug involves contacting the EVs with varying concentrations of the first drug and varying concentrations of a second drug, respectively. The method further comprises adding a probe to the EVs that have been contacted with the first or the second drug and determining the drug occupancy index at each concentration of the first or second drug. Then an IC₅₀ of the drug occupancy index of the first drug and an IC₅₀ of the drug occupancy index of the second drug are calculated. The method then determines the first drug is more potent than the second drug if the IC₅₀ of the drug occupancy index of the first drug is lower than the second drug and determines the first drug is less potent than the second drug if the IC₅₀ of the drug occupancy index of the first drug is higher than the second drug. In one illustrative example, three EGFR inhibitors, afatinib, osimertinib and erlotinib, each in an increasing concentration, are incubated with EVs. Drug occupancy index or determined using the ExoSCOPE method disclosed herein. Afatinib, osimertinib and erlotinib demonstrated IC₅₀ of 1.9 nM, 11.5 nM, 24.4 nM, which indicates that afatinib is most potent EGFR inhibitor among the three.

In some embodiments, after determining which one of the first drug and second drug is more potent, the patient is treated with the more potent drug.

### Patient Screening

Methods and compositions disclosed herein can also be used to detect a mutation in a subject that is related to drug resistance. A method of detecting the mutation in a target in a subject comprises: i) contacting an EV sample from the patient with a drug that binds the wild type target; ii) adding a probe to the EVs samples (i.e., a sample from the patient containing EVs) that have contacted with the drug, and the probe is competitive to drug in binding to a wild type target; iii) determining the IC₅₀ of drug occupancy for EVs from the patient as compared to a control EV sample expressing wild type target; and iv) determining that the subject has a mutation in the target if the IC₅₀ of drug occupancy for the EV sample from the patient is less than the IC₅₀ of drug occupancy for the control EV sample. In some embodiments, the EVs are captured on the sensor via binding to a capture agent immobilized on the sensor. In some embodiments, the capture agent is an antibody that is against one or more proteins selected from the group consisting of CD63, CD81, CD9, HER2, LAMP-1, Alix, HSP90, an Flotillin 1, a TSG101, EGFR, EpCAM, and MUC1

In one embodiment the mutation is an EGFR mutation. The method of detecting mutations in EGFR in a subject comprises: i) contacting an EV sample from the patient with a drug that targets EGFR; ii) adding a probe to the EV samples that have contacted with the drug, and the probe is capable of competing with the drug in binding to the EGFR in the EVs; iii) determining the IC₅₀ of drug occupancy for EVs from the patient as compared to a control EV sample expressing the wild type EGFR; and iv) determining that the subject has a mutation in the EGFR if the IC₅₀ of drug occupancy for the EV sample from the patient is less than the IC₅₀ of drug occupancy for the control EV sample. In some embodiments, the EVs are captured on the sensor via binding to a capture agent immobilized on the sensor. In some embodiments, the capture agent is antibody against one or more proteins selected from the group consisting of CD63, CD81, CD9, HER2, LAMP-1, Alix, HSP90, Flotillin 1, a TSG101, HER2, EGFR, EpCAM, and MUC1.

### Method of Diagnosis/Prognosis (Untreated Evs)

Also provided herein are methods and compositions for diagnosing a lung cancer in a subject. In some embodiments, the method comprises contacting a probe that binds EGFR with extracellular vesicles (EVs) from a sample obtained from the subject. In some embodiments, the probe is capable of competing with an EGFR inhibitor in binding to EGFR, wherein the EGFR inhibitor is any one of afatinib, osimertinib, erlotinib, dacomitinib, CNX2006, and WZ4002.

In some embodiments, the EVs are captured by a capture agent immobilized on a sensor, and the capture agent binds to a cancer marker that is preferentially expressed the lung cancer than normal cells. In some embodiments, the capture agent's binding to cancer marker does not substantially interfere with the binding of the probe to the cancer marker on the EVs. The method further comprises detecting the signal associated with binding of the probe to the EVs, and detecting a signal greater than a threshold indicates that subject has the a lung cancer. In some embodiments, the threshold is a signal associated with the binding of the probe to EVs from an individual that is free of the type of lung cancer under the same conditions.

EVs used for the diagnosis may be captured on the sensor before or after contacting the probe. The cancer marker that is preferentially expressed in lung cancer than normal tissue include, but are not limited to, HER2, MUC1, EpCAM, and EGFR.

### Composite Signature for Cancer Diagnosis

The ExoSCOPE technology enables detection of protein markers on the same platform, enabling biomarker discovery and direct clinical application for different diseases. For example, as disclosed above, identification of EGFR, EpCAM and MUC1 as marker combination in circulating extracellular vesicles for detecting lung cancer and providing drug occupancy signatures to distinguish treatment efficacy. In some embodiments plasma samples are obtained from the patients, and the EV subpopulations expressing two or more cancer markers selected from the group consisting of EGFR, EpCAM and MUC1 are captured on the plasmonic sensor disclosed above. A composite drug occupancy index (or a composite cancer signature) can be generated using a cross-trained regression model based on the ExoSCOPE analyses of the cancer markers and validated the model using leave-one-out cross-validation, as described above. A composition and drug occupancy index greater than a threshold indicates the patient has lung cancer. In some embodiments, the threshold is the composite drug occupancy index determined on EVs from an individual that is free of the type of lung cancer under the same conditions.

In comparison to other EV analyses (e.g., EV protein composition alone or vesicle counts) the ExoSCOPE composite cancer signature demonstrated the best accuracy for disease classification—the area under the curve (AUC) of the assay is typically at least 0.8 or at least 0.9. see (FIG. 24). In one illustrative example, the accuracy of using ExoSCOPE composite cancer signature on three putative cancer markers (EGFR, EpCAM and MUC1) as well as pan-extracellular vesicle marker (CD63) for the lung cancer diagnosis demonstrated the best accuracy, represented by area under curve AUC is 0.982, see FIG. 5b.

As described above and below in the Examples, the application discloses a robust, blood-based approach involving EVs for the molecular characterization of drug-target interactions, even of solid tumors. The ExoSCOPE platform a dedicated system for multiparametric analysis of EV drug dynamics, directly in clinical blood samples. In comparison to other analytical technologies, the ExoSCOPE not only presents distinct technology advances, but also expands the clinical reach of EVs for activity-based monitoring of drug-target interactions.

The methods and compositions related to ExoSCOPE leverage synergistic assay and sensor development and is a technology advancement in the field of monitoring drug-target interactions. In terms of assay design, the ExoSCOPE employs amplified labeling with bio-orthogonal probes; the competitive probes not only enable specific labeling of whole vesicles, but also provide reactive handles for enzymatic signal amplification in situ, to locally produce optical deposits for enhanced plasmonic sensing. In terms of sensor design, the platform supports precise spatial engineering. EVs are protein-typed and probe-amplified within the cavities of plasmonic nanoring resonators; all molecular reactions are mapped accordingly to exploit local electromagnetic hotspots. Drawing on this assay-sensor synergy, the platform achieves superior analytical performance. While drug-target interactions are commonly evaluated for drug discovery and development (i.e., on recombinant proteins, cell line and/or animal models) (Refs. 34 and 35), such measurements cannot be readily performed in patients during clinical studies. This is primarily due to the limitations of existing analytical approaches; in comparison to these technologies for quantifying drug-target interactions (e.g., thermal shift assays), which require extensive processing and a large sample amount (e.g., invasive tissue biopsies), the ExoSCOPE is sensitive and measures multiparametric drug dynamics (i.e., protein composition and drug occupancy changes) directly in a small amount of EV specimen (5 μL of clinical plasma sample in 1 hour).

For biomarker innovation, the ExoSCOPE not only reveals new insights about vesicular composition, but also introduces many clinical opportunities. In comparison to conventional EV analyses, which measure either biophysical or biochemical markers (e.g., vesicle counts or total proteins) (Shao et al. (2018)), the ExoSCOPE monitors activity-based drug dynamics and reveals integrative metrics that closely correlate to cellular drug effects (e.g., drug occupancy and potency). Unlike conventional blood pharmacologic analyses (e.g., PK/PD), which measure total drug concentration or ensemble biochemical responses in blood (i.e., lack tumor-specificity) (Tuntland et al. (2014)), the ExoSCOPE interrogates distinct subpopulations of circulating EVs to unveil cell-specific drug effects. When applied for clinical monitoring, the ExoSCOPE-developed signatures accurately reflect disease status and rapidly distinguish treatment outcomes.

With its enhanced capabilities for multiparametric evaluation of vesicle drug dynamics, the methods and compositions disclosed herein could be used to investigate complex drug interactions. Beyond the current demonstration of EGFR inhibitors, further development and incorporation of new, high-quality small molecule probes (Arrowsmith et al. (2015)) could enable multiplexed, broad-spectrum drug analysis. Likewise, comprehensive profiling in an expanded panel of EVs (e.g., oncosomes) (Meehan et al. (2016)) could provide new insights on target identification and drug selectivity. Technical improvements, through the integration of new click chemistries (Schreiber et al. (2019)), molecular assays (Ho et al., 2018 and Sundah et al., 2019) and advanced sensors (Gooding and Gaus (2016); Wu et al. (2020)), are likely to further enhance the analytical performance to enable measurements of rare interactions and reveal synergistic effects in combination therapies.

Clinically, the methods and compositions disclosed herein can be used to realize diverse medical opportunities. Specifically, multiparametric and activity-based analysis of EVs could lead to a paradigm shift in blood-based drug evaluation, particularly for targeted drug selection and real-time treatment monitoring. With its robust performance, even in clinical plasma samples, the technology could be applied to discover new EV composite signatures, across different drugs and vesicle molecular subtypes (e.g., derived from different cell origins) (Lim et al., Adv Biosyst e1900309 (2020); Lim et al., ACS Sens 5, 4-12 (2020)), in a spectrum of diseases (e.g., cancers, cardiovascular diseases, and neurological diseases). Such signatures could provide new metrics for correlating to various (un)desired drug effects (e.g., on-target potency and off-target side effects) (Borrebaeck (2017)) thereby improving patient stratification and rationalizing drug selection. Further technical advances, through the incorporation of advanced fluidics and large-scale sensor fabrication (Yeh et al. (2017); Yelleswarapu et al. (2019)), could facilitate highly parallel analysis and accelerate clinical validation.

### Embodiments

This disclosure provides the following non-limiting embodiments:

Embodiment 1. A method of measuring binding of a drug to target molecules in a subject that has been treated with a drug over a treatment period, wherein the method comprises: contacting a probe with extracellular vesicles (EVs) from samples obtained from the subject at different time points of the treatment period, wherein the probe is capable of competing with the drug in binding to the target molecules in the EVs, and detecting the binding of the probe to the EVs in the samples, wherein a decrease in the binding of the probe to the EVs as treatment period progresses indicates an increase in the binding of the drug to the target molecules in the subject.

Embodiment 2. The method of embodiment 1, where contacting the probe with EVs from the samples obtained from the subject comprises: for each sample, i) contacting the EVs from the sample with a sensor, wherein the EVs are captured to the sensor, ii) contacting the probe with the EVs captured on the sensor, wherein the probe binds to target molecules on the EVs that are not already bound by the drug, wherein the binding of the probe to the target molecules results in a signal P.

Embodiment 3. The method of embodiment 2, wherein the signal P is in situ enzymatic amplification of signal corresponding to the binding of the probe to the target molecules.

Embodiment 4. The method of embodiment 2, wherein contacting the EVs with the sensor results in a signal M, and wherein the method further comprises determining the binding of the drug to the target based on the signal P and the signal M.

Embodiment 5. The method of embodiment 4, wherein the determining the binding of the drug to the target at different time points in the treatment period comprises: determining a probe labeling index μ based on the ratio of the signal P to the signal M, normalizing the probe labeling index μ to a reference probe labeling index μ₀ to produce a normalized probe labeling index p/μ₀, wherein the reference probe labeling index μ₀ is determined on a control sample, wherein the control sample is obtained from a subject that has not been treated with the drug, determining a drug occupancy index based on the normalized probe labeling index.

Embodiment 6. The method of any of embodiment 1-5, wherein the different time points are at intervals after start of the treatment period, wherein the method comprises determining drug occupancy at each time point, and determining the drug is effective if the drug occupancy at a later time point is higher than the drug occupancy at an earlier time point.

Embodiment 7. The method of any one of embodiments 2-6, wherein the EVs are captured by binding to one or more capture agents immobilized on the sensor.

Embodiment 8. The method of embodiment 7, wherein the captured EVs comprise two or more different subpopulations, each subpopulation binding to a different capture agent immobilized on a discrete area on the sensor, thereby the captured EVs bind to two or more different capture agents, wherein the method comprises calculating a composite drug occupancy based on the drug occupancies determined for the two or more different subpopulations using a multiple linear regression model.

Embodiment 9. The method of embodiment 8, wherein the two or more different capture agents are selected from the group consisting of an anti-CD63 antibody, an CD9 antibody, an CD81 antibody, an HER2 antibody, an MUC1 antibody, an EpCAM antibody, and an EGFR antibody.

Embodiment 10. A method of comparing the potency of a first drug relative to the potency of a second drug on a subject comprising: contacting the first drug and the second drug with extracellular vesicles (EVs) obtained from a sample of the subject separately, adding a probe to the EVs that have been contacted with the first drug and to the EVs that have been contacted with the second drug, wherein the probe is capable of competing with both the first drug and the second drug in binding to the target molecules in the EVs, and detecting the binding of the probe to the EVs that have been contacted with the first drug and EVs that have been contacted with the second drug, wherein a lower binding of the probe to the EVs that have been contacted with the first drug relative to the second drug indicates that the first drug is more potent than the second drug, and wherein a higher binding of the probe to the EVs that have been contacted with the first drug relative to the second drug indicates that the first drug is less potent than the second drug in the samples.

Embodiment 11. The method of embodiment 10, wherein the contacting the EVs with varying increasing concentrations of a first drug and a second drug, contacting the EVs that have been contacted with varying increasing concentrations of the first or the second drug with a probe, determining the drug occupancy at each concentration of the first and second drug, determining an IC₅₀ of the drug occupancy of the first drug and an IC₅₀ of the drug occupancy of the second drug, determining that the first drug is more potent than the second drug if the IC₅₀ of the drug occupancy of the first drug is lower than that of the second drug, or determining the first drug is less potent than the second drug if the IC₅₀ of the drug occupancy of the first drug is higher than that of the second drug.

Embodiment 12. The method of embodiment 10-11, wherein the method further comprises treat the subject with the first drug, if the first drug is determined to be more potent than, the second drug, or treat the subject with the second drug if the second drug is determined to be more potent than the first drug..

Embodiment 13. A method of detecting mutations in EGFR in a subject, the method comprising: i) contacting an EV sample from a patient with a drug that targets the wild type EGFR, ii) adding a probe to the EVs samples that have contacted with the drug, wherein the probe is capable of competing with the drug in binding to the wild type EGFR iii) determining an IC₅₀ of drug occupancy for EVs from the patient as compared to that of control EVs expressing the wild type EGFR, and iv) determining that the subject has a mutation in the EGFR if the IC₅₀ of drug occupancy for the EV sample from the patient is less than the IC₅₀ of drug occupancy for the control EVs.

Embodiment 14. The method of any of embodiments 2-10, wherein the EVs are captured on the sensor via binding to a capture agent immobilized on the sensor.

Embodiment 15. The method of embodiment 14, wherein the capture agent is an antibody that is against one or more proteins selected from the group consisting of CD63, CD81, CD9, HER2, LAMP-1, Alix, HSP90, an Flotillin 1, a TSG101, EGFR, EpCAM, and MUC1.

Embodiment 16. A method of diagnosing a lung cancer in a subject, the method comprising: contacting a probe with extracellular vesicles (EVs) from a sample obtained from the subject, wherein the EVs are captured by a capture agent immobilized on a sensor, wherein the capture agent binds to a cancer marker on the EVs, wherein the cancer marker is preferentially expressed in lung cancer than normal cells, wherein the probe binds to EGFR on the EVs, wherein the binding of the capture agent to the cancer marker does not substantially interfere with the binding of the probe to the cancer marker on the EVs, detecting a signal associated with binding of the probe to the EVs, and determining subject has the lung cancer if the signal is greater than a control.

Embodiment 17. The method of embodiment 16, wherein the EVs are immobilized on the sensor before contacting the probe.

Embodiment 18. The method of embodiment 16, wherein the EVS are immobilized on the sensor after contacting the probe.

Embodiment 19. The method of any of embodiments 16-18, wherein the cancer marker is one or more of MUC1, EpCAM, and EGFR.

Embodiment 20. The method of any one of embodiments 16-19, wherein the capture agent is an antibody against any one or more of MUC1, EpCAM, and EGFR.

Embodiment 21. The method of any one of embodiments 16-20, wherein the probe is capable of competing with an EGFR inhibitor in binding to EGFR, wherein the EGFR inhibitor is any one of afatinib, osimertinib, erlotinib, dacomitinib, CNX2006, and WZ4002.

Embodiment 22. The method of any one of embodiments 16-21, wherein the control is a signal associated with the binding of the probe to EVs from an individual that is free of the type of cancer under the same conditions.

Embodiment 23. The method of any one of embodiments 1-22, wherein the samples are bodily fluid or tissue biopsy.

Embodiment 24. The method of embodiment 23, wherein the bodily fluid is selected from the group consisting of blood, plasma, serum, ascites, and urine.

Embodiment 25. The method of any one of embodiments 2-24, wherein the sensor is a plasmonic sensor.

Embodiment 26. The method of any one of embodiments 2-24, wherein the sensor is the sensor of embodiments 35.

Embodiment 27. The method of any one of embodiments 1-25, wherein the drug is an EGFR inhibitor.

Embodiment 28. The method of embodiment 27, wherein the EGFR inhibitor is selected from the group consisting of afatinib, osimertinib, erlotinib, dacomitinib, CNX2006, and WZ4002

Embodiment 29. A sensing element comprising nanogap structures patterned on a conductive layer that is deposited on a glass substrate, wherein the nanogap structures are patterned to form nanogaps between adjacent nanostructures, and wherein the average size of nanogap is 20 to 500 nm, wherein illumination of the nanogap structures produces a surface plasmon resonance.

Embodiment 30. The sensing element of embodiment 29, wherein the average size of the nanogaps is 20 to 500 nm.

Embodiment 31. The sensing element of embodiment 29 or 30, wherein the nanogap structures are nanorings, wherein the nanogaps are formed between an outer circular shape and an inner circular shape wherein the outer circular shape has an outer diameter in a range from 200 nm to 500 nm, and/or the inner circular shape has an inner diameter in a range from 30 nm to 250 nm.

Embodiment 32. The sensing element of embodiment any of embodiments 29-31, wherein the conductive layer comprises a material selected from the group consisting of a silver, gold, copper, titanium, aluminum, and chromium.

Embodiment 33. The sensing element of any of embodiments 29-32, wherein the nanogap structures form a periodic lattice.

Embodiment 34. The sensing element of any one of embodiments 29-33, wherein the sensing element further comprises a capture agent immobilized on the glass substrate in the nanoring gap.

Embodiment 35. A sensor comprising an array of the sensing element of any one of embodiments 29-34.

Embodiment 36. A microfluidic system comprising: a flow cell, wherein the flow cell comprises a sensor array comprising a plurality of sensing elements of embodiment 29, microfluidic channels for introducing samples into the sensor array; and a light source, wherein the light source is arranged to illuminate the sensor array.

Embodiment 37. A probe that is capable of competing with a drug in binding to its target, wherein the probe contains a tag, wherein the tag can ligate to an enzyme, and wherein the enzyme is capable of catalyzing a reaction to produce an insoluble optical product and producing a detectable signal.

Embodiment 38. The probe of embodiment 37, wherein the probe is a click probe.

Embodiment 39. The probe of embodiment 38, wherein the click probe ligates to the enzyme through a copper-free click reaction.

Embodiment 40. The probe of embodiment 37 or 38, wherein the enzyme is conjugated to tetrazine or dibenzocyclooctyne (DBCO).

Embodiment 41. The probe of any one of embodiments 37-40, wherein the enzyme is tetrazine-conjugated horseradish peroxidase (HRP).

Embodiment 42. The probe of any one of embodiments 37-41, wherein the drug is an EGFR inhibitor and its target is EGFR, and the probe has substantially similar binding and/or functional activity to the EGFR inhibitor.

Embodiment 43. The probe of embodiment 42, wherein the functional activity is an anti-proliferation activity.

Embodiment 44. The probe of any one of embodiments 38-43, wherein the click probe has a structure of

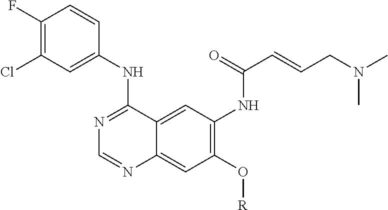

Embodiment 45. The probe of embodiment 44, wherein R contains a chemically tractable tag and a linker.

Embodiment 46. The probe of embodiment 44, wherein R is selected from the group consisting of

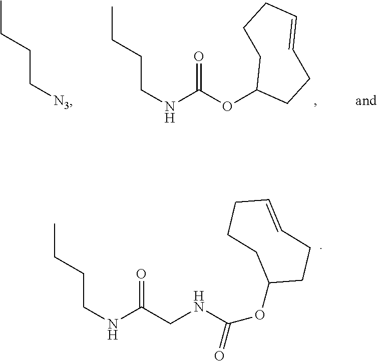

Embodiment 47. The probe of embodiment 42 or 43, wherein the EGFR inhibitor is selected from the group consisting of afatinib, osimertinib, erlotinib, dacomitinib, CNX2006, and WZ4002.

The following examples are offered for illustrative purposes and are not intended to limit the invention. Those of skill in the art will readily recognize a variety of non-critical parameters that can be changed or modified to yield essentially the same results.

### Examples

### Example 1. Methods

Chemical synthesis. All chemicals were purchased from commercial vendors and used without further purification, unless indicated otherwise. Stock solutions were prepared in DMSO and stored at −20° C. before use. Click probes were prepared as described in the detailed synthesis section (Example 8) and characterized through NMR spectra (¹H-NMR, ¹³C-NMR) on a Bruker 400 MHz/500 MHz NMR spectrometer and high-resolution electrospray ionization mass spectra (HRMS-ESI) on a Bruker microTOF-Q II at 25° C. (room temperature).

Cell culture. All human cancer cell lines were obtained from American Type Culture Collection. H3255, PC9 and H1975 cells were cultured in Roswell park memorial institute medium (RPMI-1640, Hyclone), while A431 was cultured in Dulbecco's modified eagle medium (DMEM, Hyclone), supplemented with 10% (v/v) of fetal bovine serum (FBS, Gibco) and 1% (v/v) of penicillin-streptomycin (Gibco) in a humidified 37° C. incubator with 5% CO2. All cell lines were tested and free of mycoplasma contamination (MycoAlert Mycoplasma Detection Kit, Lonza, LT07-418).

EV collection and characterization. Cells at passages 1-15 were cultured in vesicle-depleted medium (with 5% depleted FBS) for 48 hr, before vesicle collection and characterization according to MISEV guidelines (Thery et al. (2018)). All media containing EVs were filtered through a 0.2-μm membrane filter (regenerated cellulose, Millipore). For conventional analysis (e.g., Western blotting and ELISA), EVs were enriched by differential centrifugation (first at 10,000 g and subsequently at 100,000 g). For ExoSCOPE analysis, all samples were used directly for measurements. All EV samples were stored at −80° C. before further analysis. For independent characterization of vesicle concentration and size distribution, we used the nanoparticle tracking analysis (NTA) system (Nanosight, NS300) embedded with a 488-nm laser and sCMOS camera. All samples were first diluted to contain an estimated concentration of ˜10⁸-10⁹ vesicles/mL, to match NTA's counting compatibility in this concentration range. For each sample, NTA experiments were performed in triplicates, collecting 60 s videos with a minimum of 200 valid tracks recorded per video. For each counting experiment, we manually adjusted the camera focus to achieve optimal particle visualization. Camera level setting was maintained at 10 and detection threshold at 5, for maximal sensitivity with minimal background noise. The field of view was adjusted to contain ˜50 vesicles. All collected videos were used directly for statistical analyses of particle counts and size distribution.

ExoSCOPE sensor design. We performed full three-dimensional, finite-difference time-domain (FDTD) simulations to optimize the sensor design (FDTD solutions, Lumerical). Periodic boundary conditions in x- and y-directions were used to simulate an infinite array of periodic nanorings. Nanoring arrays with different periodicities and geometries were illuminated with a plane wave from the bottom side. A non-uniform mesh with a minimum grid size of 2 nm was applied. In determining the optimal sensor geometry (FIG. 9a), we used the spectral shift in response to global and local refractive index changes, respectively.

Sensor fabrication. The optimized ExoSCOPE sensor design was fabricated on 2.5×2.5 cm glass substrate. First, the substrate was spin-coated (4000 r.p.m. for 70 s) with a 180-nm layer PMMA 495 k, followed by hard baking on a hotplate at 170° C. for 5 min. A second 180-nm layer PMMA 495 k was spin-coated (4000 r.p.m. for 70 s) onto the substrate, and post baked at 180° C. for 2 min. A thin layer of Espacer was then applied to the surface to improve the substrate conductivity. Subsequently, electron-beam lithography (EBL, Jeol 6300FS) was performed to define the nanoring array pattern in the resist, before development in organic solvent (1:3 v/v methyl isobutyl ketone MIBK and isopropyl alcohol IPA) for 45 s. After rinsing with IPA, an adhesion layer (Ti/Au; 5 nm/50 nm) was deposited onto the substrate, through electron-beam physical vapor deposition (AJA E Beam Evaporator System). This was followed by a lift-off process in solvent stripper (MicroChem Remover PG). All nanoring dimension, thickness and sensor uniformity were characterized by scanning electron microscopy (JEOL 6701) and atomic force microscopy (Bruker Dimension FastScan).

Channel assembly. A standard soft lithography was used for the fabrication of a multichannel flow-cell. First, SU-8 negative resist (SU-8 2050, Microchem) was spin-coated on a Si wafer at 3500 r.p.m. for 30 s. The resist was then baked at 65° C. and 95° C. for 1 and 6 min, respectively. After UV light exposure, the resist was baked again before being developed under agitation. The developed wafer was rinsed with IPA and dried by nitrogen. The SU-8 mold was chemically treated by trichlorosilane vapor inside a desiccator for 15 min. Polydimethylsiloxane polymer (PDMS) and cross-linker were mixed at a ratio of 10:1 and casted onto the SU-8 mold and cured in an oven at 75° C. for 30 min. Finally, the PDMS layer was cut from the mold and assembled onto the ExoSCOPE sensor. All inlets and outlets were made with 1.1-mm biopsy punch for sample processing.

Optical setup and analysis. For experimental analysis, a tungsten halogen lamp (StockerYale Inc.) was used to illuminate the ExoSCOPE sensor through a 10× microscope objective. Transmitted light was collected by an optical fiber and fed into a spectrometer (Ocean Optics). All measurements were performed at room temperature, in an enclosed box to eliminate ambient light interference. The transmitted light intensity was digitally recorded in counts against wavelength. For spectral analysis, the spectral peaks were determined using a custom-built R program by fitting the transmission peak using local regression method.

In-ring surface functionalization. To evaluate the location effect of molecular reactions on the ExoSCOPE detection signal, we performed differential surface functionalization to achieve bioconjugation within the nanoring gap (in-ring, SiO2) and on top of the sensor surface (atop, Au), respectively. For in-ring functionalization, the fabricated sensor was first treated with oxygen plasma to activate the dangling OH groups on SiO2 surface and improve the surface uniformity for conjugation. After treatment, the cleaned sensor was immersed into a 2% solution of (3-Aminopropyl)triethoxysilane (APTES) in ethanol for 15 min, rinsed and dried in an oven at 100° C. for 5 min. The APTES-modified sensor was washed in PBS and treated with 2.5% (v/v) glutaraldehyde in PBS for 10 min at room temperature. Following a rinse, the sensor was reacted with 0.1 mg/ml of capture antibodies in PBS buffer at room temperature for 15 min. Comparatively, for atop functionalization, the sensor was incubated in a mixture of long active (carboxylated) thiol-PEG and short inactive methylated thiol-PEG (1:3 active: inactive, 10 mM in PBS) to enable S—Au interaction. The modified sensor was washed in PBS and activated through carbodiimide crosslinking, in a mixture of excess NHS/EDC dissolved in MES buffer, and reacted with capture antibodies as described above. All surface modifications were spectrally monitored. All conjugated sensors were stored in PBS at 4° C. for subsequent use.

ExoSCOPE assay. We developed the ExoSCOPE assay for the direct analysis of both marker composition and drug occupancy in EVs. Samples were diluted 1:1 in PBS before ExoSCOPE analyses. We treated the diluted samples with click probe A3 (tagged with TCO, 100 nM) at 37° C. for 30 min before analysis. For competitive drug binding, EVs were incubated with varying concentrations of drugs (e.g., afatinib, erlotinib, osimertinib, from 100×DMSO stock) or vehicle control (DMSO) for 10 min prior to probe treatment and ExoSCOPE analysis. To measure EV marker composition (for a single marker analysis), we incubated the samples on antibody-functionalized ExoSCOPE sensors (1 μL, 10 min, room temperature), to achieve marker-specific EV capture. As a control experiment, sensors functionalized with IgG isotope control antibodies were used. Relative spectral changes (plasmonic signals) were measured to determine marker-specific EV binding. To measure EV drug occupancy, we used the click probes within sensor-bound vesicles to achieve enzymatic amplification. Specifically, we utilized the probes' click handles (TCO) to recruit tetrazine-conjugated horseradish peroxidase (HRP), which catalyzed in situ, enzymatic conversion of the soluble substrate (3,3′-diaminobenzidine, DAB) to form local, insoluble polymeric products on labeled vesicles; these insoluble deposits acted as optical products to amplify the plasmonic signals. Experimentally, to prepare tetrazine-conjugated HRP, high sensitivity streptavidin-conjugated HRP (BD Biosciences) was reacted with tetrazine-biotin (1 μM) in PBS at room temperature for 30 min and desalted through a Zeba column (Thermo Scientific). For in situ probe amplification, sensor-bound vesicles were fixed with 4% paraformaldehyde and permeabilized with 0.1% Triton X-100 (5 min, room temperature), before being incubated with the purified tetrazine-RP (5 min, room temperature). After removing the unbound HRP, we incubated the reaction with a mixture of DAB and hydrogen peroxide (Life Technologies) as the reaction substrate (5 min, room temperature) to achieve enzymatic development of insoluble optical deposits. Relative spectral changes were measured to determine probe labeling. All flow rates for incubation and washing were maintained at 3 μL/min and 10 μL/min, respectively. Localized deposition of insoluble enzymatic products was confirmed through scanning electron microscopy. To evaluate performance, all experiments were also accompanied with quality controls (batch positive controls) to ensure successful antibody and probe labeling. The optimized workflow and analysis are illustrated in FIG. 7.

ExoSCOPE analysis. Based on spectral measurements, we defined EV probe labeling index (μ) and drug occupancy index (ξ) as follows.

For ExoSCOPE analysis with targeting antibody against marker m:
μₘ=Pₘ/Mₘ; where Mₘ=marker-induced EV binding signal, relative to a sample-matched control that was incubated with IgG isotope control antibody.

Pₘ=probe-induced amplification signal, relative to a sample-matched control treated with analog probe without the click group.
ξₘ=1−μm/μm⁰; where μm⁰ refers to that of a matched control not treated with the drug.

Probe molecular modeling. Studies were performed by using Autodock Vina as previously reported (Bianco et al. (2016)). The crystal structure of EGFR kinase in complex with afatinib (PDB ID: 4G5J) was used for the modeling, and the active site was defined by the bound ligand. Covalent docking was performed by using flexible side chain method at the site of Cys797. The results were presented by PyMOL (version 2.3.2).

Probe labeling in cells. Cells were cultured in EV-depleted medium for 48 hr to reach ˜80% confluency, and then treated with a click probe (100 nM, from 200 μM DMSO stock) for 1 hr in a humidified 37° C. incubator with 5% CO2. For competitive labeling, cells were treated with a drug (from 1,000×DMSO stock) or vehicle control (DMSO) for 1 hr prior to probe addition. After incubation, cells were washed twice with PBS, collected by scraping and centrifugation. The pellets were used directly for cell validation experiments, as described below.

In-gel fluorescence analysis of cell labeling. Cell pellets were resuspended in Pierce IP lysis buffer (Thermo Scientific) supplemented with protease inhibitor cocktail (Thermo Scientific). After lysis on ice, the lysate was centrifuged at 2000 g, 4° C. for 2 min, and the supernatant was collected and normalized to 1.5 mg/mL with PBS. 40 μL of each sample was first blocked with iodoacetamide (15 mM, Sigma) for 30 min at room temperature in the dark, followed by click reaction with either DBCO-TMR or tetrazine-TMR (25 μM, Click Chemistry Tools) at room temperature for 30 min. The sample was then boiled in LDS sample buffer at 95° C. for 5 min, and resolved by 4-12% SDS-PAGE gel. Finally, the fluorescently labeled proteins were visualized with a ChemiDoc imaging system and subsequently stained with Bio-Safe Coomassie (Bio-Rad).

Click ELISA. For cell studies, 1 μg of probe-labeled lysates diluted in 50 μL of PBS were reacted with tetrazine-biotin (1 μM) at room temperature for 1 hr. The reaction was then diluted with 50 μL of PBS and added to antibody-coated plate, prepared as described in the ELISA section (see below for details). After incubation for 1 hr at room temperature and washing, streptavidin-conjugated HPR was added and incubated for 1 hr at room temperature. Finally, chemiluminescent substrate Luminol and hydrogen peroxide (Thermo Scientific) were added and the signal was recorded by a microplate reader (Tecan). For EV studies, 10 μL of samples containing different concentrations of vesicles in PBS were prepared and labeled with probe A3, as described above. The reaction was then diluted with 90 μL of PBS and added to antibody-coated plate for 1 hr at room temperature. Following washing of the unbound vesicles, tetrazine-conjugated HRP was added and incubated for 1 hr before chemiluminescence signal development (Luminol).

Real-time binding kinetics. Cell pellets were resuspended in PBS and lysed by probe sonication (Q700, Fisherbrand) in an ice bath. Lysates were then centrifuged at 2000 g, 4° C. for 2 min. The supernatant was collected and diluted to 4 mg/mL in PBS supplemented with 0.1% NP-40 and 1 mM DTT. Binding kinetics of the probe A3 to EGFR was measured through biolayer interferometry (Pall Fortebio). In brief, 100 nM of biotinylated A3 (prepared by reacting A3 with tetrazine-biotin) were immobilized onto streptavidin-functionalized interferometry sensors. After a brief washing step, the loaded biosensors were incubated for 500 s with cell lysate solutions, each with distinct EGFR expression level and/or mutation status, to measure different probe binding. This was followed by another washing step. All binding data (changes in biolayer optical thickness) were in a continuous manner, to determine real-time binding kinetics.

Flow cytometry. We performed flow cytometry analysis to validate probe labeling of cells and EVs, respectively. For cellular studies, cells incubated with probes were fixed with BD Phosflow Fix Buffer I (BD Biosciences) for 30 min. Cell suspensions were labeled with 5 μg/mL of primary antibodies for 1 hr at 4° C. Following centrifugation and washing, cells were labeled with 2 μg/mL of FITC-conjugated secondary antibody (BD Biosciences) for 30 min at 4° C. and washed twice by centrifugation. For visualization of the click probes, cells were finally labeled with 100 nM tetrazine-Cy5 for 5 min at room temperature and washed twice by centrifugation, before flow cytometry cell analysis.

For EV analysis, vesicles were first incubated with the click probes at 37° C. for 1 h. The mixture was added to functionalized polystyrene beads. For bead functionalization, streptavidin-coated 3.0 m polystyrene beads (Spherotech) was incubated with biotinylated anti-CD63 antibodies (10 g/mL, BD Biosciences) in PBS with 0.5% bovine serum albumin (BSA, Sigma) overnight at 4° C. The mixture was washed and resuspended in PBS with 0.5% BSA, before being applied for EV capture. For visualization of the click probes, the bead-captured vesicles were labeled with 100 nM tetrazine-Cy5 for 5 min at room temperature and washed. For all flow cytometry analysis, FITC and APC fluorescence were assessed using a CytoFLEX Flow Cytometer (Beckman Coulter). Mean fluorescence intensities of all cells/beads, excluding debris, was determined using FlowJo (version 10.6.1), and biomarker expression levels were normalized against isotype control antibodies while probe expression levels were normalized against no-drug no-probe control.

Western blotting. Cells or EVs isolated by ultracentrifugation were lysed in radioimmunoprecipitation assay (RIPA) buffer containing protease inhibitors (ThermoFisher Scientific, Waltham, MA) and quantified using bicinchoninic acid assay (BCA assay, ThermoFisher Scientific, Waltham, MA). Protein lysates (10 μg) were resolved by sodium dodecyl sulfate polyacrylamide gel electrophoresis (SDS-PAGE), transferred onto polyvinylidene fluoride membrane (PVDF, Invitrogen, Carlsbad, CA), and immunoblotted with antibodies against protein markers: EGFR (Cell Signaling, Danvers, MA), CD63 (Santa Cruz Biotechnology Dallas, TX), LAMP-1 (BD Biosciences, San Jose, CA), Alix (Cell Signaling), HSP90 (Cell Signaling), Flotillin 1 (BD Biosciences), TSG101 (BD Biosciences), GM130 (Cell Signaling), Calnexin (BD Biosciences), phopho-EGFR (Y1068, Cell Signaling), phospho-Gab1 (Y621, Cell Signaling), phospho-PLC71 (Y783, Cell Signaling), phospho-Akt (S473, Cell Signaling), phospho-Shc (Y239/240, Cell Signaling), actin (Cell Signaling), and GAPDH (Cell Signaling). Following incubation with horseradish peroxidase-conjugated secondary antibody (Cell Signaling), enhanced chemiluminescence was used for immunodetection (Thermo Scientific).

Enzyme-linked immunosorbent assay (ELISA). Capture antibodies (5 μg/mL) were adsorbed onto ELISA plates (ThermoFisher Scientific) and blocked in PBS containing 1% BSA (Sigma, St. Louis, MO) before incubation with samples. After washing with PBST (PBS with 0.05% Tween 20), detection antibodies (2 μg/mL) were added and incubated for 2 hr at room temperature. Following incubation with horseradish peroxidase-conjugated secondary antibody (ThermoFisher Scientific) and chemiluminescent substrate (Thermo Scientific), chemiluminescence intensity was measured for protein detection (Tecan, Mannedorf, Switzerland).

MTS assay. Different targeted drugs (afatinib, osimertinib, erlotinib, dacomitinib, CNX2006, and WZ4002; Selleckchem) were used to investigate the effects of treatment on cell proliferation. To determine the half maximal inhibition of proliferation (GI50) of the drug, cells were seeded at a density of 20,000 cells per well in a 96-well plate overnight, and treated with the drug or vehicle control (final concentration of 0.1% DMSO) for 3 days. Cell viability was assessed using the [3-(4,5-dimethylthiazol-2-yl)-5-(3-carboxymethoxyphenyl)-2-(4-sulfophenyl)-2H-tetrazolium inner salt (MTS) cell proliferation assay (Promega).

Scanning electron microscopy. All samples were fixed with half-strength Karnovsky's fixative and washed twice with PBS. After dehydration in a series of increasing ethanol concentrations, samples were transferred for critical drying (Leica) and subsequently sputter-coated with gold (Leica), before imaging with a scanning electron microscope (JEOL 6701).

Transmission electron microscopy. All samples were fixed with 2% paraformaldehyde, permeabilized with 0.01% triton and transferred onto a copper grid (Ted Pella). The bound samples were labeled with the click probe conjugated to gold nanoparticle (Ted Pella, 10 nm), washed and contrast-stained with uranyl oxalate and methyl cellulose mixture. Dried samples were imaged with a transmission electron microscope (JEOL 2200FS).

Clinical samples. The study was approved by National University Hospital (NUH) and National Cancer Centre Singapore (NCCS) Institutional Review Board (2007/430/B, 2016/01201 and 2019/00711). All subjects were recruited according to IRB-approved protocols after obtaining informed consent. A total of 163 blood samples (from 106 individuals) were evaluated in this study (Table 1). To determine the diagnostic performance of the ExoSCOPE platform, we obtained lung cancer samples (n=46 patients) and control blood samples (n=30 control individuals). Cancer diagnoses were established from gold-standard pathology reports.

For the study on longitudinal treatment monitoring, patients were recruited into the trial based on the following selection criteria: patients had received and failed a first-line chemotherapy; prior chemotherapy was finished at least four weeks before starting this study; patients did not receive any other tyrosine kinase inhibitors previously. To evaluate the ExoSCOPE's ability to monitor drug occupancy and treatment efficacy, longitudinal blood samples were collected from the same patients before and during treatment (n=30 patients). Tumor measurements were determined by computed tomography (CT) and treatment response was evaluated using the Response Evaluation Criteria in Solid Tumors (RECIST) criteria. For plasma collection, venous blood (5 ml) was drawn from subjects in EDTA tubes and processed immediately. Briefly, all blood samples were centrifuged for 10 min at 400 g (4° C.). Plasma was transferred without disturbing the buffy coat and centrifuged again for 10 min at 1100 g (4° C.). All plasma samples were de-identified and stored at −80° C. before ExoSCOPE measurements.

Clinical ExoSCOPE measurements. All plasma samples were measured according to the assay workflow outlined in FIG. 7. For multiplexed measurements, 5 μL of plasma samples were thawed on ice and mixed with the click probe (100 nM) at 37° C. for 30 min. The samples were then incubated with ExoSCOPE sensors functionalized with respective antibodies: EGFR (Merck), EpCAM (R&D Systems), MUC1 (Fitzgerald) and CD63 (BD Biosciences). All ExoSCOPE measurements were performed directly, without requiring any vesicle purification or isolation. Relative spectral changes were measured to determine EV marker composition. All samples were fixed with 4% paraformaldehyde and permeabilized with 0.1% Triton X-100 for 5 min at room temperature, before the ExoSCOPE enzymatic amplification to determine EV probe labeling and drug occupancy. For all measurements, we included a sample-matched negative control (IgG isotype control), as described previously. All ExoSCOPE measurements were performed blinded from clinical diagnoses and treatment evaluation.

Pharmacokinetics analysis. Plasma concentrations of erlotinib were quantified through a liquid chromatography tandem mass spectrometry method. Briefly, a liquid-liquid extraction of 50 μL of plasma samples was performed in a mixture of ethyl acetate and n-hexane (8/2, v/v), before liquid chromatography coupled through an electro spray interface to a tandem mass spectrometry in positive mode detection. The chromatographic separation was achieved through a C18 column (Thermo Scientific) with a mobile phase consisting of 2 mM ammonium acetate: methanol (20:80, v/v). The lower limit of detection for erlotinib was 10.4 ng/mL and the range for linearity 10.4-2510.8 ng/mL.

Statistical analysis. All measurements were performed in triplicate, and the data displayed as mean±standard deviation. Significance tests were performed via a two-tailed Student's t test. For inter-sample comparisons, multiple pairs of samples were each tested, and the resulting P values were adjusted for multiple hypothesis testing using Bonferroni correction. An adjusted P<0.05 was determined as significant. Correlation analysis was performed with Pearson's R to determine the goodness of fit in linear regressions. We further verified the agreement with Bland-Altman analysis. For clinical analysis, we used the ExoSCOPE measurements to develop multiple linear regression scoring models for classifying disease diagnosis and treatment response, respectively. For diagnostic classification, we recorded the categorical cancer and normal samples to a binary scale and use it as the outcome variable in linear regression. Power calculation (type I and type II errors at 5%) was performed to ensure the clinical sample size has sufficient power to examine different markers and marker combinations. For treatment response evaluation, we used clinical RECIST analysis as the outcome variable. To avoid overfitting and evaluate performance, we conducted leave-one-out cross-validation. Receiver operating characteristic (ROC) curves were plotted based on the regression scorings. Detection sensitivity, specificity and accuracy were determined using standard formulas. Statistical analyses were performed using R (v.3.5.0) and GraphPad Prism (v.7.0c).

### Example 2. Exoscope Analysis of Targeted Drug Occupancy in EVs

Upon targeted drug binding (e.g., small molecule inhibitors), plasma membrane protein receptors (e.g., EGFR) are internalized into cells. This recycling pathway overlaps with the formation of EVs (Tan et al. (2016)). During EV biogenesis, protein receptors are secreted into the extracellular space through nanoscale vesicles (FIG. 1a). Multimodal characterization of vesicles derived from lung cancer cells (H3255) not only confirmed their vesicular morphology and molecular composition, but also demonstrated the presence of drug-bound protein receptors in EVs (FIG. 6). We thus reasoned that EVs could serve as a reflective circulating biomarker of drug dynamics, and developed the ExoSCOPE platform to evaluate EV drug occupancy as well as cellular treatment effects (FIG. 1a).

The ExoSCOPE leverages competitive target labeling by bio-orthogonal click probes and their amplified detection to measure EV drug changes (FIG. 1a). All molecular reactions are spatially patterned within plasmonic nanoring resonators for sensitive detection (FIG. 1b). Specifically, EVs are protein-typed and probe-amplified within plasmonic sensors. For protein measurement, EVs are immuno-captured onto functionalized sensors. For probe amplification, EVs with a low drug occupancy are extensively labeled with click probes. We use the probes' bio-orthogonal handles (trans-cyclooctene, TCO) to recruit enzymes (tetrazine-conjugated horseradish peroxidase, HRP), which catalyze in situ conversion of the soluble substrate (3,3′-diaminobenzidine, DAB) to form local, insoluble deposits on the labeled vesicles (FIG. 7A). These high-density optical deposits, formed in low-drug-occupancy vesicles, lead to plasmonic signal enhancement and a red shift in the resultant transmission optical spectrum. These molecular reactions (i.e., EV protein typing and probe amplification) are spatially patterned and monitored in real time by plasmonic resonators, to enable multiparametric analysis of EV dynamics (i.e., protein composition and drug occupancy changes) (FIG. 7B).

FIG. 1C shows the characterization of a designed ExoSCOPE click probe, a TCO derivative of the EGFR-inhibitor afatinib. Molecular modeling showed the probe's specific interaction with the EGFR kinase active site, identical to that of the parent drug. Transmission electron microscopy analysis further confirmed that the probe could achieve in situ vesicle labeling (FIG. 1C, inset). We thus applied the bio-orthogonal probe for amplified labeling of EVs; through differential material functionalization (FIG. 8), we spatially patterned the molecular reactions within periodic arrays of plasmonic nanoring resonators (FIG. 1D), so as to exploit local electromagnetic hotspots within the nanoring gaps for sensitive detection (inset). Across multiple key steps in the ExoSCOPE workflow (i.e., nanoring fabrication, antibody functionalization and EV capture), the optimized sensors showed superior and uniform performance (FIGS. 9 and 10). Employing the developed ExoSCOPE platform, we measured longitudinal changes of drug occupancy in tumor-associated EVs, directly from blood samples of lung cancer patients undergoing targeted therapy (FIG. 1E). As compared to conventional blood pharmacologic analysis (e.g., PK/PD), the ExoSCOPE revealed distinct EV signatures to rapidly distinguish treatment outcomes.

### Example 3. Design and Characterization of Click Probes

In developing the ExoSCOPE platform, we first designed and prepared a panel of bio-orthogonal click probes (i.e., A1, A2 and A3) (FIG. 2A). Using EGFR as the model protein receptor, the probes were synthesized based on the core structure of afatinib (BIBW2992) (Li et al. (2008)) to confer specific covalent binding to the EGFR kinase site (FIG. 11A-B, see Example 8 for synthesis details). Each click probe contains a chemically tractable tag (i.e., azide or TCO) (Jewett & Bertozzi (2010); Patterson et al. (2014)) to enable label visualization through rapid copper-free bio-orthogonal ligation (FIG. 12A-B). To retain the native properties of afatinib and minimize interference with target binding, we prepared A1 and A2 by inserting at the 7-position (Lanning (2014)) a short, three-carbon linker and a small tag. Probe A3 was subsequently prepared by introducing a glycine moiety to A2. In comparison to the hydrophobic TCO (A2), this addition not only improved probe A3's lipophilicity (calculated distribution coefficient at pH 7.4, cLogD=3.4), but also enhanced its anti-proliferation activity (GI₅₀, on human lung cancer cells H3255) (FIG. 2B), possibly due to the improved linker flexibility (Rutkowska et al. (2016)).

We next evaluated the labeling performance of the prepared click probes. H3255 cells were incubated with the respective probes, with or without afatinib. After cell lysis, the lysates were reacted with different visualization agents (i.e., dibenzocyclooctyne (DBCO)- or tetrazine-conjugated fluorescent dyes) before electrophoresis. In-gel fluorescence imaging confirmed that among the developed probes, A3 not only demonstrated selective, afatinib-competitive labeling of EGFR, but also showed the best signal-to-noise ratio across all labeling reactions (FIG. 2C and FIG. 13A-13D). To validate probe A3's direct interactions with various EGFR proteins (e.g., wild type and mutants), we employed biolayer interferometry and monitored probe-protein binding in real time, using cell lysates known to overexpress different EGFR mutations. Probe A3 demonstrated differential binding kinetics to various EGFR mutants (FIG. 2D); high binding potentials (Bₘₐₓ/K_{d}) were observed for distinct EGFR mutants (i.e., L858R in H3255 cells and ex19del in PC9 cells) over EGFR wild-type proteins (A431 cells), in agreement with the reported mutant selectivity of the parent drug (Li et al., (2008)). Furthermore, probe A3 demonstrated rapid and specific live-cell labeling of EGFR (>90% efficiency in 15 mins) (FIG. 14A-B), making it an ideal candidate to evaluate drug-target engagement in live cells. Competitive labeling of H3255 cells with various concentrations of afatinib showed drug dose-dependent decrease in probe A3 labeling, as independently validated by in-gel fluorescence, flow cytometry and click ELISA analysis, respectively (FIG. 2E and FIG. 14C-D). The IC₅₀ values derived thereof are in the similar range (1.4-1.6 nM), consistent with the cellular activity of afatinib (FIG. 2C). Due to its specific measurement of EGFR-drug engagement, we selected probe A3 for subsequent development of the ExoSCOPE platform.

### Example 4. Amplified Detection of EV Drug Occupancy

To apply probe A3 for in situ analysis of EV drug occupancy, we first examined its ability to directly label vesicular EGFR in whole EVs. EVs were immobilized onto microbeads through anti-CD63 capture (Shao et al. (2012); Shao et al. (2015)) and incubated with probe A3, with or without afatinib. Multimodal analyses not only confirmed in situ probe labeling, that is afatinib-competitive and specific to vesicular EGFR (FIG. 3A and FIG. 6D), but also demonstrated effective probe amplification, through enzymatic deposition of insoluble optical products (FIG. 15). We next developed spatially-optimized plasmonic nanoring resonators (FIG. 1D and FIG. 9A-D) to locally measure EV probe-labeling (FIG. 3B). In comparison to our published configurations on plasmonic nanoholes (Im et al. (2014); Lm et al. (2019)), we found that periodic lattices of gold nanorings could improve spatial control for signal amplification (FIG. 16A-B). Specifically, field simulations showed that enhanced electromagnetic fields are located within the nanoring gap (in-ring), as compared to that on the sensor surface (atop) (FIG. 3b, left). To leverage this in-ring field confinement, we patterned and performed ExoSCOPE molecular reactions (i.e., EV capture and probe amplification) within the nanoring gaps (FIG. 8A-B). Scanning electron micrographs confirmed the spatial distribution of the bound vesicles as defined by the differential surface modifications (FIG. 3B, right). Specifically, for the in-ring functionalization, ˜85% of the bound targets were located within the nanoring gaps (FIG. 17A). In agreement with the numerical simulations (FIG. 16A-C), experimental sensorgrams further confirmed the superior performance of the in-ring detection (FIG. 3B, right and FIG. 17A-B).

Using the optimized ExoSCOPE assay (in-ring functionalization), we next investigated its analytical performance. We conducted experiments with probe-labeled EVs to evaluate the ExoSCOPE platform against an ELISA-based approach (FIG. 18A-B). The ExoSCOPE assay showed a limit of detection (LOD) of ˜1,000 probe-labeled EVs, which is 10⁴-fold better than that of the click ELISA assay (FIG. 3C). Importantly, the ExoSCOPE measurements showed a high specificity, even when performed directly in plasma samples (FIG. 18C-D). To account for differences in vesicle counts and composition across samples, we leveraged the ExoSCOPE's ability to measure both marker-specific EV binding (M) as well as probe-induced amplification signal (P) in the same vesicles (FIG. 7A-B), and defined the probe labeling index (μ=P/M) to evaluate the average probe density per sensor-captured vesicle. Using Evs derived from various cell lines with known EGFR expression (FIG. 18E), we used the ExoSCOPE platform to measure their respective and confirmed that the analyses indeed reflect vesicular EGFR expression levels (FIG. 3D and FIG. 18F).

We finally established the ExoSCOPE platform to evaluate drug occupancy in Evs. We incubated Evs with an increasing concentration of respective EGFR inhibitors (e.g., covalent: afatinib, osimertinib; non-covalent: erlotinib), before ExoSCOPE analysis. We further defined the drug occupancy index (ξ=1−μ/μ⁰, where μ⁰ refers to that of a matched control not treated with the drug), to quantify relative drug-target engagement in EVs (FIG. 7A-B). The resultant EV analysis (ξ_{EV}) demonstrated an IC₅₀ of 1.9 nM, 11.5 nM and 24.4 nM, for afatinib, osimertinib and erlotinib, respectively (FIG. 3E). These results match closely to that of the independent analysis on cellular drug occupancy (ξ_{cell}), where cells were treated with competitive drugs (FIG. 19A-B, IC₅₀ of ξ_{cell}=1.7, 13.1 and 26.2 nM), and are in good agreement with published studies (Refs 23 and 31). Importantly, the ξ_{EV} analysis also showed mutant selectivity; Evs with EGFR mutant proteins demonstrated a lower IC₅₀ than those with wild-type (FIG. 3F). Such trends are consistent with the cellular analyses (FIG. 2D and FIG. 19C-D). These agreements not only suggest that vesicular EGFR are good surrogates for cellular proteins, but also demonstrate that the ExoSCOPE platform could sensitively assess EV drug occupancy to reveal delicate changes of drug interaction with mutant proteins.

### Example 5. Multiplexed Exoscope for Evaluating Drug Potency

To evaluate if EV drug occupancy could be used as a good circulating biomarker of ongoing cell treatment, even in heterogeneous cell mixtures, we developed the multiplexed ExoSCOPE platform. In this study, we treated cells with targeted inhibitors and measured time-dependent drug occupancy in secreted Evs (ξ_{EV}) as well as in parent cells undergoing treatment ξ_{cell}) (FIG. 4A). For EV analysis, using different antibodies to selectively capture vesicles, we developed the multiplexed ExoSCOPE workflow to measure protein composition and drug occupancy in molecular subpopulations of Evs. For cellular analysis, we used flow cytometry for cell marker gating and occupancy measurements.

Using cancer cell lines that express different levels of putative cancer markers (FIG. 20A), we treated a heterogeneous cell mixture with targeted treatment (erlotinib, 1 μM) or drug vehicle (DMSO). We applied the multiplexed ExoSCOPE analysis to conduct real-time ξEV analysis, through pan-EV (CD63) as well as cancer marker-based (EGFR, EpCAM and MUC1) (Sandfeld-Paulsen et al., 2016) vesicle analysis (FIG. 4B). Within 24 hours after treatment initiation, across all vesicle subpopulations examined, ξ_{EV} increased and showed a good agreement with the longitudinal cell data of parent cells (FIG. 20B). Minimal changes were observed with respect to EV concentration and EGFR expression (FIG. 21A-C). Time-dependent analyses with different drug treatments (i.e., different inhibitors at different concentrations) (FIG. 4C) further confirmed the strong correlation between ξ_{EV} and corresponding cell (FIG. 4D and FIG. 22A-D), indicating that ExoSCOPE analysis can reflect cellular drug occupancy in real time.

We next assessed the relationship between drug occupancy and potency. Using H3255 cell culture treated with a panel of EGFR inhibitors (10 nM for 3 hours), we compared the ξ_{EV} analysis with the corresponding drug potency (GI₅₀) in cells (FIG. 4E). The results showed an inverse relationship between the two parameters: drugs which caused a high ξ_{EV} (>0.6) exhibited sub-nanomolar potency in cells (<1 nM), while those with a low ξ_{EV} (<0.2) showed less effective GI₅₀ (>30 nM). Finally, we expanded the ξ_{EV}/GI₅₀ analysis to other cell lines expressing different EGFR mutations (FIG. 4F). We observed two sets of linear relationships, which could distinguish sensitive vs. resistant treatment (FIG. 23A-E). Specifically, at a similar cellular GI₅₀, a higher ξ_{EV} was observed in resistant cell lines, indicating their need for a higher drug occupancy to achieve a comparable potency. These findings not only validated the multiplexed ExoSCOPE analysis, but also indicated the correlation of ξ_{EV} drug occupancy to cellular potency, thereby potentiating its use as a circulating surrogate in reporting treatment outcome.

### Example 6. Clinical Analysis of Lung Cancer Therapy

To evaluate the clinical applications of the ExoSCOPE platform, we conducted a feasibility study using lung cancer as a model system. We aimed at addressing (1) if multiplexed ExoSCOPE could be directly applied to clinical plasma samples, (2) the accuracy of ExoSCOPE in detecting cancer patients, and (3) whether ExoSCOPE-revealed drug occupancy signatures could distinguish treatment efficacy. We first assessed the diagnostic capability of the ExoSCOPE platform. We obtained plasma samples from lung cancer patients as well as control individuals (n=76, Table 1). Using clinical plasma samples, we performed multiplexed ExoSCOPE analysis on three putative cancer markers (EGFR, EpCAM and MUC1) as well as an EV marker (CD63) (FIG. 5A). We further developed a cross-trained regression model based on the ExoSCOPE analyses of the cancer markers and validated the model using leave-one-out cross-validation. In comparison to other EV analyses (e.g., EV protein composition alone or vesicle counts) (FIG. 24A-F), the ExoSCOPE composite cancer signature demonstrated the best accuracy for disease classification (FIG. 5B, area under curve AUC=0.982).

To evaluate treatment efficacy, we next applied the multiplexed ExoSCOPE to examine time-dependent changes in EV drug occupancy. Blood samples were collected from lung cancer patients undergoing targeted treatment of EGFR inhibitor (erlotinib at 100 mg daily)33, at various time points (T0, baseline, before treatment; T1, day−1, 24 hours after initial treatment; and T2, day−8, 192 hours after treatment initiation). Employing the multiplexed ExoSCOPE platform, we used the identified cancer markers to measure longitudinal changes in EV drug occupancy (Δξ) as well as changes in EV protein marker composition (ΔM) in different vesicle subpopulations (FIG. 5C, top). We also performed conventional blood pharmacologic analysis to measure total drug concentration in blood plasma (ΔD) (FIG. 5C, bottom). Responder and non-responder status was clinically determined at the end of the treatment (day−21) by tumor volumetric imaging. Interestingly, only A analysis showed distinguishing trends between clinical responders and non-responders; the difference could be observed as early as in T1 (24 hours after treatment initiation, FIG. 25A), while the other analyses could not (FIG. 25B-C).

Motivated by this early indicative potential of EV drug dynamics, we expanded the multiplexed ExoSCOPE analysis to include more patients for longitudinal treatment monitoring (n=30 patients, Table 1). Specifically, based on multiplexed changes in EV drug occupancy at T1 (ΔξT1, 24 hours), we computed a composite score (Iξ) through a cross-trained regression model (FIG. 5D). Likewise, we analyzed the respective changes in EV marker composition at T1 (ΔMT1 and I_{M}) as well as corresponding changes in plasma drug concentration (ΔDT1). Of these metrics, only EV drug occupancy changes (Iξ) could effectively distinguish responders from non-responders (P<0.0001) (FIG. 5E). The other changes showed insignificant differences between the two clinical groups. Importantly, the ExoSCOPE classification based on EV drug dynamics was accurate (FIG. 26A) and correlated well with clinical patient survival data (FIG. 26B-C), indicating the effectiveness of the technology for early monitoring of targeted treatment outcomes.

TABLE 1
Clinical information

| | Diagnostic assessment | | | Treatment monitoring | | | |
|---|---|---|---|---|---|---|---|
| | (FIG. 5a-b) | | | (FIG. 5c-d) | | | |
| | Number (%) | | | Number (%) | | | |
| | Lung cancer | | Healthy | | | Non- | |
| Characteristics | patients | | controls | Responders | | responders | |
| Individuals | 46 | | 30 | 10 | | 20 | |
| Total plasma samples | 46 | | 30 | 30 | | 57 | |
| Gender | | | | | | | |
| Male | 23 | (50%) | 15 (50%) | 2 | (20%) | 13 | (65%) |
| Female | 23 | (50%) | 15 (50%) | 8 | (80%) | 7 | (35%) |
| Age | | | | | | | |
| Median | 58 | | 55 | 57 | | 55 | |
| Range | 26-73 | | 26-79 | 39-72 | | 33-73 | |
| Histology subtype | | | | | | | |
| Adenocarcinoma | 23 | (50%) | NA | 7 | (70%) | 12 | (60%) |
| Squamous cell carcinoma | 6 | (13%) | NA | 2 | (20%) | 4 | (20%) |
| Large cell carcinoma | 2 | (4%) | NA | — | | — | |
| Others | 15 | (33%) | NA | 1 | (10%) | 4 | (20%) |
| Smoking history | | | | | | | |
| Non-smoker | 28 | (61%) | NA | 8 | (80%) | 12 | (60%) |
| Ex-smoker | 9 | (20%) | NA | 1 | (10%) | 6 | (30%) |
| Current smoker | 5 | (11%) | NA | 1 | (10%) | 2 | (10%) |
| Not known | 4 | (8%) | NA | — | | — | |
| Stage | | | | | | | |
| I | 10 | (22%) | NA | — | | — | |
| II | 4 | (9%) | NA | — | | — | |
| III/IV | 30 | (65%) | NA | 10 | (100%) | 20 | (100%) |

| | | | | | |
|---|---|---|---|---|---|
| Not known | 2 | (4%) | NA | — | — |

| | | | | |
|---|---|---|---|---|
| Vesicle concentration | 2.58 ± 1.33 | 2.56 ± 1.27 | 2.33 ± 1.37 | 2.81 ± 1.28 |
| (×10¹⁰/mL) | | | | |

### Example 8. Detailed Synthesis and NMR Characterization of Click Probes

A work flow of synthesis of A1, A2, and A3 are shown in FIG. 11A-B. General information. All chemicals were purchased from commercial vendors and used without further purification, unless indicated otherwise. All reactions requiring anhydrous conditions were carried out under nitrogen atmosphere using oven-dried glassware. See FIG. 11A. Reactions were monitored by analytical thin layer chromatography (TLC) on pre-coated silica gel plates (Merck 60 F254) and spots were visualized by UV (254/365 nm). Flash column chromatography was carried out using 200 to 400 mesh silica gel. All NMR spectra (¹H-NMR, ¹³C-NMR) were recorded on a Bruker 400 MHz/500 MHz NMR spectrometer. Chemical shifts are reported in parts per million referenced with respect to appropriate internal standards or residual solvent peaks (CDCl₃=7.26 ppm, CD30D=3.31 ppm, DMSO-d₆=2.50 ppm). See FIG. 11B. The following abbreviations were used in reporting spectra: br (broad singlet), s (singlet), d (doublet), t (triplet), q (quartet), m (multiplet), dd (doublet of doublets), coupling constant (J, Hz). Prep-HPLC was conducted on Gilson Prep-HPLC system using reverse-phase Phenomenex Luna 5 m C18(2) 100 Å 50×30.0 mm column. High-resolution electrospray ionization mass spectra (HRMS-ESI) were obtained on a Bruker microTOF-Q II. All measurements were performed at room temperature (RT) of 25° C.

### tert-Butyl (3-((4-((3-chloro-4-fluorophenyl)amino)-6-nitroquinazolin-7-yl)oxy)propyl) carbamate (compound 2)

To a solution of tert-butyl (3-hydroxypropyl)carbamate (1.21 g, 6.9 mmol) in anhydrous THE (7.5 mL), NaH (60% dispersion in mineral oil, 276 mg, 6.9 mmol) was added under a nitrogen atmosphere. The reaction was stirred at room temperature for 30 minutes until gas evolution ceased. Then to it was added a solution of compound 1 (1.01 g, 3 mmol; Bepharm) in anhydrous THE (7.5 mL) dropwise in 1 hr using a syringe pump. The reaction was stirred at room temperature for an additional 3 hrs and then quenched with aqueous saturated NaHCO₃ (5 mL). The mixture was diluted with EtOAc (50 mL) and washed with aqueous saturated NaHCO₃, brine and dried over Na2SO4. After evaporation, the residue was purified by silica gel chromatography (EtOAc:Hex=2:1) to produce the desired product as a yellow solid (750 mg, 51%).

¹H NMR (400 MHz, DMSO) δ 10.15 (s, 1H), 9.21 (s, 1H), 8.67 (s, 1H), 8.16 (dd, J=6.8, 2.5 Hz, 1H), 7.88-7.71 (m, 1H), 7.50-7.42 (m, 2H), 6.91 (t, J=5.3 Hz, 1H), 4.30 (t, J=6.1 Hz, 2H), 3.12 (q, J=6.6 Hz, 2H), 1.90 (p, J=6.4 Hz, 2H), 1.36 (s, 9H). ¹³C NMR (101 MHz, DMSO) δ 157.68, 157.22, 155.61, 154.73, 153.91, 153.19, 152.31, 138.72, 135.93, 123.57, 122.31 (d, J=6.8 Hz), 121.73, 118.85 (d, J=18.4 Hz), 116.51 (d, J=21.2 Hz), 110.12, 107.76, 77.51, 67.51, 36.68, 28.77, 28.20. HRMS (ESI) calculated for [M+H]+: 492.1450, found: 492.1445.

### CAN-tert-butyl (3-((4-((3-chloro-4-fluorophenyl)amino)-6-(4-(dimethylamino)but-2-enamido) quinazolin-7-yl)oxy)propyl)carbamate (compound 3)

To a solution of compound 2 (600 mg, 1.22 mmol) in acetone (30 mL), aqueous saturated NH4Cl (6 mL) as added, followed by zinc dust (638 mg, 9.76 mmol) under a nitrogen atmosphere. The reaction was stirred at room temperature for 2 hrs and then filtered. The solid was washed with EtOAc. The organics were washed with aqueous saturated NaHCO₃, and the aqueous layer was extracted with EtOAc. The combined organics were dried over Na2SO4 and evaporated to afford tert-butyl (3-((6-amino-4-((3-chloro-4-fluorophenyl)amino)quinazolin-7-yl)oxy)propyl)carbamate as a light yellow foam (570 mg), which was used in the next step without purification. HRMS (ESI) calculated for [M+H]+: 492.1450, found: 492.1445.

To a solution of the residue above in anhydrous THE (15 mL), DIEA (1.05 mL, 6 mmol) was added, followed by a solution of CAN-4-bromobut-2-enoyl chloride (1.44 mmol, freshly prepared according to the reported procedure described in Lanning et al. (2014). in THE (10 mL) under a nitrogen atmosphere. The reaction was stirred at room temperature overnight. Upon the addition of silica gel and evaporation, it was purified by silica gel chromatography (MeOH:CH2Cl2=5%) to afford a mixture of CAN-tert-butyl (3-((6-(4-bromobut-2-enamido)-4-((3-chloro-4-fluorophenyl)amino)quinazolin-7-yl)oxy)propyl)carbamate and CAN-tert-butyl (3-((4-((3-chloro-4-fluorophenyl)amino)-6-(4-chlorobut-2-enamido)quinazolin-7-yl)oxy)propyl) carbamate as a light yellow foam (822 mg), which was used in the next step without further purification.

To a solution of the mixture above in DMF (6 mL), Me2NH (3 mL, 6 mmol, 2M in THF) and Cs2CO3 (977 mg, 3 mmol) were added. The reaction was heated to 70° C. for 1 hr. The THF was removed by evaporation, and the salt was removed by filtration. Upon evaporation to dryness, the residue was dissolved in MeOH/DMSO and purified by semi-preparative HPLC (ACN:water+0.01% TFA) to afford desired product forming salt with TFA (380 mg, 45% over 3 steps) as a viscous yellow oil.

¹H NMR (400 MHz, MeOD) δ 9.00 (s, 1H), 8.63 (s, 1H), 7.80 (dd, J=6.6, 2.6 Hz, 1H), 7.57 (ddd, J=8.9, 4.1, 2.7 Hz, 1H), 7.30 (s, 1H), 7.17 (t, J=8.9 Hz, 1H), 7.06-6.95 (m, 1H), 6.87 (d, J=15.2 Hz, 1H), 4.26 (t, J=5.6 Hz, 2H), 4.02 (d, J=6.8 Hz, 2H), 3.30-3.23 (m, 2H), 2.97 (s, 1H), 2.94 (s, 6H), 2.09-2.00 (m, 2H), 1.37 (s, 9H). ¹³C NMR (101 MHz, MeOD) δ 164.45, 163.00 (q, J=34.5 Hz), 159.92, 158.62, 158.34, 157.39, 155.88, 150.67, 138.74, 134.93 (d, J=3.5 Hz), 133.60 (d, J=13.1 Hz), 130.88, 127.14, 125.29 (d, J=7.3 Hz), 121.50 (d, J=18.8 Hz), 119.56, 117.56 (d, J=11.1 Hz), 116.08 (d, J=115.3 Hz), 108.13, 101.09, 80.03, 68.60, 58.76, 43.23, 29.72, 28.73. HRMS (ESI) calculated for [M+H]+: 573.2392, found: 573.2389.

### CAN-N-(7-(3-azidopropoxy)-4-((3-chloro-4-fluorophenyl)amino)quinazolin-6-yl)-4-(dimethylamino)but-2-enamide (A1)

To a solution of compound 3 (50 mg, 0.073 mmol) in CH2Cl2 (1 mL), TFA (0.1 mL) was added. The reaction was stirred at room temperature for 1 hr. Upon evaporation to dryness, the residue was used in next step without purification.

To a solution of the residue above in MeOH (0.5 mL) and water (0.5 mL), K2CO3 (41 mg, 0.292 mmol) and CuSO4 (1 mg, 6.3 μmol) were added, followed by a solution of TfN3 (0.11 mmol, freshly prepared according to the reported procedure described in (Liu & Tor (2003) in CH2Cl2 (1 mL). Then MeOH (1 mL) was added to make a homogeneous solution. The reaction was stirred in dark at room temperature overnight. The mixture was diluted with EtOAc (5 mL) and washed with aqueous saturated NaHCO₃ (2 mL). The aqueous layer was extracted with EtOAc (5 mL), and the combined organics were dried over Na2SO4. After evaporation, the residue was purified by semi-preparative HPLC (ACN:water) to afford the desired product as a white solid (24 mg, 65% over 2 steps).

¹H NMR (400 MHz, DMSO) δ 9.80 (s, 1H), 9.48 (s, 1H), 8.90 (s, 1H), 8.53 (s, 1H), 8.13 (dd, J=6.9, 2.6 Hz, 1H), 7.80 (ddd, J=9.1, 4.3, 2.7 Hz, 1H), 7.42 (t, J=9.1 Hz, 1H), 7.29 (s, 1H), 6.81 (dt, J=15.4, 6.0 Hz, 1H), 6.55 (d, J=15.4 Hz, 1H), 4.28 (t, J=6.0 Hz, 2H), 3.62 (t, J=6.8 Hz, 2H), 3.10 (dd, J=6.0, 1.1 Hz, 2H), 2.19 (s, 6H), 2.11 (t, J=6.4 Hz, 2H). ¹³C NMR (126 MHz, DMSO) δ 163.58, 153.89, 142.08, 125.69, 123.49, 122.38, 122.33, 116.33, 107.27, 65.87, 59.74, 47.66, 45.11, 27.77. HRMS (ESI) calculated for [M+H]+: 499.1768, found: 499.1775.

### CAN-cyclooct-3-en-1-yl (3-((4-((3-chloro-4-fluorophenyl)amino)-6-(CAN-4-(dimethylamino)but-2-enamido)quinazolin-7-yl)oxy)propyl)carbamate (A2)

To a solution of compound 3 (50 mg, 0.073 mmol) in CH2C12 (1 mL), TFA (0.1 mL) was added. The reaction was stirred at room temperature for 1 hr. Upon evaporation to dryness, the residue was used in subsequent synthesis without purification.

To a solution of the residue above in DMF (2 mL), TCO-NHS (23 mg, 0.088 mmol) and DIEA (52 μL, 0.292 mmol) were added under a nitrogen atmosphere. The reaction was stirred in dark at room temperature overnight. The mixture was diluted with EtOAc (5 mL) and washed with water (2 mL). The water layer was extracted with EtOAc (5 mL), and the combined organics were washed with brine and dried over Na2SO4. After evaporation, the residue was purified by silica gel chromatography (MeOH:CH2Cl2=10%) to afford the desired product as a white solid (25 mg, 55% over 2 steps).

¹H NMR (400 MHz, DMSO) δ 9.80 (s, 1H), 9.43 (s, 1H), 8.95 (s, 1H), 8.52 (s, 1H), 8.12 (dd, J=6.8, 2.4 Hz, 1H), 7.87-7.67 (m, 1H), 7.40 (t, J=9.1 Hz, 1H), 7.25 (s, 1H), 7.11 (t, J=5.5 Hz, 1H), 6.82 (dt, J=15.3, 6.0 Hz, 1H), 6.58 (d, J=15.4 Hz, 1H), 5.62-5.48 (m, 1H), 5.47-5.33 (m, 1H), 4.22 (t, J=6.1 Hz, 2H), 4.19 (t, J=5.1 Hz, 1H), 3.20 (d, J=5.8 Hz, 2H), 3.09 (d, J=5.9 Hz, 2H), 2.28-2.20 (m, 3H), 2.19 (s, 6H), 2.00-1.91 (m, 2H), 1.90-1.76 (m, 4H), 1.67-1.47 (m, 3H). ¹³C NMR (126 MHz, DMSO) δ 163.57, 156.78, 156.03, 154.25, 154.13, 153.81, 152.20, 148.84, 142.19, 136.85, 134.94, 132.48, 127.41, 125.83, 123.52, 122.38 (d, J=6.6 Hz), 118.69 (d, J=18.3 Hz), 116.42 (d, J=21.6 Hz), 115.45, 108.85, 107.28, 79.14, 72.29, 66.18, 60.06 (d, J=57.0 Hz), 40.70, 39.52, 38.23, 36.90, 33.74, 32.17, 30.59, 28.46. HRMS (ESI) calculated for [M+H]+: 625.2700, found: 625.2718.

### (E)-cyclooct-4-en-1-yl (2-((3-((4-((3-chloro-4-fluorophenyl)amino)-6-((E)-4-(dimethylamino)but-2-enamido)quinazolin-7-yl)oxy)propyl)amino)-2-oxoethyl)carbamate (A3)

To a solution of TCO-NHS (38 mg, 0.14 mmol) in THE (0.75 mL), a solution of glycine (14 mg, 0.14 mmol) in water (0.25 mL) was added, followed by Et3N (58 μL, 0.42 mmol). The reaction was stirred in dark at room temperature overnight. Upon evaporation to dryness, the residue was used in the next step without purification.

To a solution of compound 3 (50 mg, 0.073 mmol) in CH2Cl2 (1 mL), TFA (0.1 mL) was added. The reaction was stirred at room temperature for 1 hr and dried through evaporation. To this residue, a solution of the crude TCO-Gly-COOH in DMF (1 mL) was added. Then a mixture of EDC HCl (27 mg, 0.14 mmol) and DIEA (49 μL, 0.28 mmol) was added, in ice bath under a nitrogen atmosphere. The reaction was stirred in dark at 0° C. for 30 minutes and subsequently at room temperature for 4 hrs. The mixture was directly purified by semi-preparative HPLC (ACN:water) to prepare the desired product as a white solid (21 mg, 42% over 2 steps).

¹H NMR (400 MHz, DMSO) δ 9.80 (s, 1H), 9.51 (s, 1H), 8.94 (s, 1H), 8.52 (s, 1H), 8.13 (dd, J=6.9, 2.6 Hz, 1H), 7.95 (t, J=5.5 Hz, 1H), 7.80 (ddd, J=9.0, 4.3, 2.7 Hz, 1H), 7.41 (t, J=9.1 Hz, 1H), 7.26 (s, 1H), 7.11 (t, J=5.9 Hz, 1H), 6.82 (dt, J=15.4, 6.0 Hz, 1H), 6.60 (d, J=15.4 Hz, 1H), 5.65-5.43 (m, 1H), 5.45-5.27 (m, 1H), 4.23 (t, J=6.2 Hz, 2H), 4.19-4.10 (m, 1H), 3.55 (d, J=4.9 Hz, 2H), 3.31 (d, J=6.6 Hz, 2H), 3.09 (d, J=5.3 Hz, 2H), 2.30-2.19 (m, 3H), 2.19 (s, 6H), 2.03-1.95 (m, 2H), 1.93-1.72 (m, 4H), 1.67-1.43 (m, 3H). ¹³C NMR (126 MHz, DMSO) δ 169.45, 163.55, 156.75, 156.06, 154.34, 154.08, 153.78, 152.15, 148.84, 142.09, 136.84, 134.84, 132.47, 127.40, 125.81, 123.48, 122.34 (d, J=6.9 Hz), 118.65 (d, J=18.4 Hz), 116.40 (d, J=21.4 Hz), 115.59, 108.82, 107.30, 79.47, 66.02, 59.75, 45.11, 43.64, 40.61, 39.76, 38.11, 35.10, 33.70, 32.08, 30.54, 28.20. HRMS (ESI) calculated for [M+H]+: 682.2914, found: 682.2932.

### REFERENCES

1. Moscow, J. A., Fojo, T. & Schilsky, R. L. The evidence framework for precision cancer medicine. Nat Rev Clin Oncol 15, 183-192 (2018).
2. Eisenhauer, E. A. et al. New response evaluation criteria in solid tumours: revised RECIST guideline (version 1.1). Eur J Cancer 45, 228-247 (2009).
3. Litière, S., Collette, S., de Vries, E. G., Seymour, L. & Bogaerts, J. RECIST—learning from the past to build the future. Nat Rev Clin Oncol 14, 187-192 (2017).
4. Basu, A. et al. An interactive resource to identify cancer genetic and lineage dependencies targeted by small molecules. Cell 154, 1151-1161 (2013).
5. Jonas, O. et al. An implantable microdevice to perform high-throughput in vivo drug sensitivity testing in tumors. Sci Transl Med 7, 284ra57 (2015).
6. Martinez Molina, D. et al. Monitoring drug target engagement in cells and tissues using the cellular thermal shift assay. Science 341, 84-87 (2013).
7. Jones, L. H. & Neubert, H. Clinical chemoproteomics-Opportunities and obstacles. Sci Transl Med 9, eaaf7951 (2017).
8. Gerry, C. J. & Schreiber, S. L. Chemical probes and drug leads from advances in synthetic planning and methodology. Nat Rev Drug Discov 17, 333-352 (2018).
9. Shao, H. et al. New Technologies for Analysis of Extracellular Vesicles. Chem Rev 118, 1917-1950 (2018).
10. Colombo, M., Raposo, G. & Thery, C. Biogenesis, secretion, and intercellular interactions of exosomes and other extracellular vesicles. Annu Rev Cell Dev Biol 30, 255-289 (2014).
11. van Niel, G., D'Angelo, G. & Raposo, G. Shedding light on the cell biology of extracellular vesicles. Nat Rev Mol Cell Biol 19, 213-228 (2018).
12. Minciacchi, V. R., Freeman, M. R. & Di Vizio, D. Extracellular vesicles in cancer: exosomes, microvesicles and the emerging role of large oncosomes. Semin Cell Dev Biol 40, 41-51 (2015).
13. Mathieu, M., Martin-Jaular, L., Lavieu, G. & Thery, C. Specificities of secretion and uptake of exosomes and other extracellular vesicles for cell-to-cell communication. Nat Cell Biol 21, 9-17 (2019).
14. Shao, H. et al. Protein typing of circulating microvesicles allows real-time monitoring of glioblastoma therapy. Nat Med 18, 1835-1840 (2012).
15. Choi, D. S., Kim, D. K., Kim, Y. K. & Gho, Y. S. Proteomics of extracellular vesicles: Exosomes and ectosomes. Mass Spectrom Rev 34, 474-490 (2015).
16. Valadi, H. et al. Exosome-mediated transfer of mRNAs and microRNAs is a novel mechanism of genetic exchange between cells. Nat Cell Biol 9, 654-659 (2007).
17. Skog, J. et al. Glioblastoma microvesicles transport RNA and proteins that promote tumour growth and provide diagnostic biomarkers. Nat Cell Biol 10, 1470-1476 (2008).
18. Maas, S. L. N., Breakefield, X. O. & Weaver, A. M. Extracellular Vesicles: Unique Intercellular Delivery Vehicles. Trends Cell Biol 27, 172-188 (2017).
19. Wang, Z. et al. Dual-selective magnetic analysis of extracellular vesicle glycans. Matter 2, 150-166 (2020).
20. Tan, X., Lambert, P. F., Rapraeger, A. C. & Anderson, R. A. Stress-Induced EGFR Trafficking: Mechanisms, Functions, and Therapeutic Implications. Trends Cell Biol 26, 352-366 (2016).
21. Xin, H., Namgung, B. & Lee, L. P. Nanoplasmonic optical antennas for life sciences and medicine. Nat Rev Mater 3, 228-243 (2018).
22. Ligler, F. S. & Gooding, J. J. Lighting Up Biosensors: Now and the Decade To Come. Anal Chem 91, 8732-8738 (2019).
23. Li, D. et al. BIBW2992, an irreversible EGFR/HER2 inhibitor highly effective in preclinical lung cancer models. Oncogene 27, 4702-4711 (2008).
24. Jewett, J. C. & Bertozzi, C. R. Cu-free click cycloaddition reactions in chemical biology. Chem Soc Rev 39, 1272-1279 (2010).
25. Patterson, D. M., Nazarova, L. A. & Prescher, J. A. Finding the right (bioorthogonal) chemistry. ACS Chem Biol 9, 592-605 (2014).
26. Lanning, B. R. et al. A road map to evaluate the proteome-wide selectivity of covalent kinase inhibitors. Nat Chem Biol 10, 760-767 (2014).
27. Rutkowska, A. et al. A Modular Probe Strategy for Drug Localization, Target Identification and Target Occupancy Measurement on Single Cell Level. ACS Chem Biol 11, 2541-2550 (2016).
28. Shao, H. et al. Chip-based analysis of exosomal mRNA mediating drug resistance in glioblastoma. Nat Commun 6, 6999 (2015).
29. Im, H. et al. Label-free detection and molecular profiling of exosomes with a nano-plasmonic sensor. Nat Biotechnol 32, 490-495 (2014).
30. Lim, C. Z. J. et al. Subtyping of circulating exosome-bound amyloid R reflects brain plaque deposition. Nat Commun 10, 1144 (2019).
31. Cross, D. A. et al. AZD9291, an irreversible EGFR TKI, overcomes T790M-mediated resistance to EGFR inhibitors in lung cancer. Cancer Discov 4, 1046-1061 (2014).
32. Sandfeld-Paulsen, B. et al. Exosomal proteins as prognostic biomarkers in non-small cell lung cancer. Mol Oncol 10, 1595-1602 (2016).
33. Hirsch, F. R. et al. Lung cancer: current therapies and new targeted treatments. Lancet 389, 299-311 (2017).
34. Hoelder, S., Clarke, P. A. & Workman, P. Discovery of small molecule cancer drugs: successes, challenges and opportunities. Mol Oncol 6, 155-176 (2012).
35. Wagner, J. A. Strategic approach to fit-for-purpose biomarkers in drug development. Annu Rev Pharmacol Toxicol 48, 631-651 (2008).
36. Tuntland, T. et al. Implementation of pharmacokinetic and pharmacodynamic strategies in early research phases of drug discovery and development at Novartis Institute of Biomedical Research. Front Pharmacol 5, 174 (2014).
37. Arrowsmith, C. H. et al. The promise and peril of chemical probes. Nat Chem Biol 11, 536-541 (2015).
38. Meehan, B., Rak, J. & Di Vizio, D. Oncosomes—large and small: what are they, where they came from. J Extracell Vesicles 5, 33109 (2016).
39. Schreiber, C. L. & Smith, B. D. Molecular conjugation using non-covalent click chemistry. Nat Rev Chem 3, 393-400 (2019).
40. Ho, N. R. Y. et al. Visual and modular detection of pathogen nucleic acids with enzyme-DNA molecular complexes. Nat Commun 9, 3238 (2018).
41. Sundah, N. R. et al. Barcoded DNA nanostructures for the multiplexed profiling of subcellular protein distribution. Nat Biomed Eng 3, 684-694 (2019).
42. Gooding, J. J. & Gaus, K. Single-Molecule Sensors: Challenges and Opportunities for Quantitative Analysis. Angew Chem Int Ed Engl 55, 11354-11366 (2016).
43. Wu, X. et al. Exosome-templated nanoplasmonics for multiparametric molecular profiling. Sci Adv 6, eaba2556 (2020).
44. Lim, C. Z. J., Natalia, A., Sundah, N. R. & Shao, H. Biomarker Organization in Circulating Extracellular Vesicles: New Applications in Detecting Neurodegenerative Diseases. Adv Biosyst e1900309 (2020).
45. Lim, C. Z. J., Zhang, L., Zhang, Y., Sundah, N. R. & Shao, H. New Sensors for Extracellular Vesicles: Insights on Constituent and Associated Biomarkers. ACS Sens 5, 4-12 (2020).
46. Borrebaeck, C. A. Precision diagnostics: moving towards protein biomarker signatures of clinical utility in cancer. Nat Rev Cancer 17, 199-204 (2017).
47. Yeh, E. C. et al. Self-powered integrated microfluidic point-of-care low-cost enabling (SIMPLE) chip. Sci Adv 3, e1501645 (2017).
48. Yelleswarapu, V. et al. Mobile platform for rapid sub-picogram-per-milliliter, multiplexed, digital droplet detection of proteins. Proc Natl pAcad Sci USA 116, 4489-4495 (2019).
49. Thery, C. et al. Minimal information for studies of extracellular vesicles 2018 (MISEV2018): a position statement of the International Society for Extracellular Vesicles and update of the MISEV2014 guidelines. J Extracell Vesicles 7, 1535750 (2018).
50. Bianco, G., Forli, S., Goodsell, D. S. & Olson, A. J. Covalent docking using autodock: Two-point attractor and flexible side chain methods. Protein Sci 25, 295-301 (2016).
51. Lanning, B. R. et al. A road map to evaluate the proteome-wide selectivity of covalent kinase inhibitors. Nat. Chem. Biol. 10, 760-767 (2014).
52. Liu, Q. & Tor, Y. Simple conversion of aromatic amines into azides. Org. Lett. 5, 2571-2572 (2003).
53. Csizmadia F, Tsantili-Kakoulidou A, Panderi I, Darvas F. Prediction of distribution coefficient from structure. 1. Estimation method. J. Pharm. Sci. 86: 865-871 (1997).
54. Jain, P. K. & El-Sayed, M. A. Plasmonic coupling in noble metal nanostructures. Chemical Physics Letters (2010).
55. Hugall, J. T., Singh, A. & Hulst, N. F. V. Plasmonic cavity coupling. Acs Photonics (2018).
56. Sammut C., Webb G.I. (eds) Encyclopedia of Machine Learning. Springer, Boston, MA. doi.org/10.1007/978-0-387-30164-8_469.
57. Cocucci E, Meldolesi J Ectosomes and exosomes: shedding the confusion between extracellular vesicles. Trends Cell Biol. 2015; 25(6):364-372.
58. Kumar A, Petri E T, Halmos B, Boggon T J. Structure and clinical relevance of the epidermal growth factor receptor in human cancer. J Clin Oncol. 2008, 26, 1742-1751.

### INCORPORATION BY REFERENCE

Each and every publication and patent document referred to in this disclosure is incorporated herein by reference in its entirety for all purposes to the same extent as if each such publication or document was specifically and individually indicated to be incorporated herein by reference.

While the invention has been described with reference to the specific examples and illustrations, changes can be made and equivalents can be substituted to adapt to a particular context or intended use as a matter of routine development and optimization and within the purview of one of ordinary skill in the art, thereby achieving benefits of the invention without departing from the scope of what is claimed and their equivalents.

1. A method of measuring binding of a drug to target molecules in a subject that has been treated with a drug over a treatment period, wherein the method comprises:
contacting a probe with extracellular vesicles (EVs) from samples obtained from the subject at different time points of the treatment period, wherein the probe is capable of competing with the drug in binding to the target molecules in the EVs, and
detecting the binding of the probe to the target molecules in the EVs in the samples, wherein a decrease in the binding of the probe to the EVs as treatment period progresses indicates an increase in the binding of the drug to the target in the subject.

2. The method of claim 1, where contacting the probe with EVs from the samples obtained from the subject comprises: for each sample,
i) contacting the EVs from the sample with a sensor, wherein the EVs are captured to the sensor, and
ii) contacting the probe with the EVs captured on the sensor, wherein the probe binds to target molecules on the EVs that are not already bound by the drug,
wherein the binding of the probe to the target molecules results in a signal P.

3. The method of claim 2, wherein the signal P is in situ enzymatic amplification of signal corresponding to the binding of the probe to the target molecules.

4. The method of claim 2, wherein contacting the EVs with the sensor results in a signal M, and wherein the method further comprises determining the binding of the drug to the target based on the signal P and the signal M.

5. The method of claim 4, wherein the determining the binding of the drug to the target at different time points in the treatment period comprises:
determining a probe labeling index μ based on the ratio of the signal P to the signal M,
normalizing the probe labeling index μ to a reference probe labeling index μ0 to produce a normalized probe labeling index μ/μ0,
wherein the reference probe labeling index μ0 is determined on a control sample,
wherein the control sample is obtained from a subject that has not been treated with the drug,
determining a drug occupancy index based on the normalized probe labeling index.

6. The method of claim 5, wherein the different time points are at intervals after start of the treatment period,
wherein the method comprises determining drug occupancy at each time point, and determining the drug is effective if the drug occupancy at a later time point is higher than the drug occupancy at an earlier time point.

7. The method of claim 2, wherein the EVs are captured by binding to one or more capture agents immobilized on the sensor.

8. The method of claim 7, wherein the captured EVs comprise two or more different subpopulations, each subpopulation binding to a different capture agent immobilized on a discrete area on the sensor, thereby the captured EVs bind to two or more different capture agents,
wherein the method comprises calculating a composite drug occupancy based on the drug occupancies determined for the two or more different subpopulations using a multiple linear regression model.

9. A method of diagnosing a lung cancer in a subject, the method comprising:
contacting a probe with extracellular vesicles (EVs) from a sample obtained from the subject,
wherein the EVs are captured by a capture agent immobilized on a sensor,
wherein the capture agent binds to a cancer marker on the EVs, wherein the cancer marker is preferentially expressed in lung cancer than normal cells,
wherein the probe binds to EGFR on the EVs,
wherein the binding of the capture agent to the cancer marker does not substantially interfere with the binding of the probe to the cancer marker on the EVs,
detecting a signal associated with binding of the probe to the EVs, and
determining subject has the lung cancer if the signal is greater than a control.

10. The method of claim 9, wherein the EVs are immobilized on the sensor before contacting the probe or wherein the EVs are immobilized on the sensor after contacting the probe.

11. The method of claim 9, wherein the cancer marker is selected from the group consisting of MUC1, EpCAM, and EGFR, and/or wherein the capture agent is an antibody against any one or more of MUC1, EpCAM, and EGFR.

12. The method of claim 9, wherein the probe is capable of competing with an EGFR inhibitor in binding to EGFR, wherein the EGFR inhibitor is any one of afatinib, osimertinib, erlotinib, dacomitinib, CNX2006, and WZ4002.

13. The method of claim 2, wherein the sensor is any one of the sensor of claim 19.

14. The method of claim 1, wherein the drug is an EGFR inhibitor.

15. A sensing element comprising nanogap structures patterned on a conductive layer that is deposited on a glass substrate,
wherein the nanogap structures are patterned to form nanogaps between adjacent nanostructures, and wherein the average size of nanogap is 20 to 500 nm, and
wherein illumination of the nanogap structures produces a surface plasmon resonance.

16. The sensing element of claim 15, wherein the average size of the nanogaps is 20 to 500 nm.

17. The sensing element of claim 15, wherein the nanogap structures are nanorings, wherein the nanogaps are formed between an outer circular shape and an inner circular shape wherein the outer circular shape has an outer diameter in a range from 200 nm to 500 nm, and/or the inner circular shape has an inner diameter in a range from 30 nm to 250 nm.

18. The sensing element of claim 15, wherein the sensing element further comprises a capture agent immobilized on the glass substrate in the nanogaps.

19. A sensor comprising an array of the sensing element of claim 15.

20. A probe that is capable of competing with a drug in binding to its target, wherein the probe contains a tag, wherein the tag can ligate to an enzyme, and
wherein the enzyme is capable of catalyzing a reaction to produce an insoluble optical product and producing a detectable signal.

21. The probe of claim 20, wherein the enzyme is conjugated to tetrazine or dibenzocyclooctyne (DBCO), or a tetrazine-conjugated horseradish peroxidase (HRP).

22. The probe of claim 20, wherein the drug is an EGFR inhibitor and its target is EGFR, and the probe has substantially similar binding and/or functional activity to the EGFR inhibitor.

23. The probe of claim 20, wherein the click probe has a structure of [chemical structure]
